(19) United States
(12) Patent Application Publication
(10) Pub. No.: US 2025/0163118 A1
(43) Pub. Date: May 22, 2025

(54) INTERLEUKIN-2/INTERLEUKIN-2 RECEPTOR ALPHA FUSION PROTEINS AND METHODS OF USE

(71) Applicant: University of Miami, Miami, FL (US)

(72) Inventor: Thomas MALEK, Coral Gables, FL (US)

(21) Appl. No.: 19/031,119

(22) Filed: Jan. 17, 2025

Related U.S. Application Data
(62) Division of application No. 17/822,333, filed on Aug. 25, 2022.
Application No. 17/822,333 is a division of application No. 15/501,392, filed on Feb. 2, 2017, now Pat. No. 12,084,483.
(60) Provisional application No. 62/033,726, filed on Aug. 6, 2014.

(51) Int. Cl. C07K 14/55; A61K 38/00; C07K 14/715

(52) U.S. Cl. CPC C07K 14/55; C07K 14/7155; A61K 38/00; C07K 2319/00; C07K 2319/21

(57) ABSTRACT

Various methods and compositions are provided which can be employed to modulate the immune system. Compositions include a fusion protein comprising: (a) a first polypeptide comprising Interleukin-2 (IL-2) or a functional variant or fragment thereof; and (b) a second polypeptide, fused in frame to the first polypeptide, wherein the second polypeptide comprises an extracellular domain of Interleukin-2 Receptor alpha (IL-2Rα) or a functional variant or fragment thereof, and wherein the fusion protein has IL-2 activity. Various methods are provided for modulating the immune response in a subject comprising administering to a subject in need thereof a therapeutically effective amount of the IL-2/IL-2Rα fusion protein disclosed herein.

### STATEMENT OF FEDERAL FUNDING

This invention was made with government support under grant number R01 DK093866, awarded by the National Institute of Health (NIH), National Institute of Diabetes and Digestive and Kidney Diseases (NIDDK). The government has certain rights in the invention.

### FIELD OF THE INVENTION

The presently disclosed subject matter generally relates to methods and compositions for modulating the immune response employing an Interleukin-2/Interleukin-2 Receptor alpha fusion protein.

### REFERENCE TO A SEQUENCE LISTING SUBMITTED AS A TEXT FILE VIA EFS-WEB

The official copy of the sequence listing is submitted concurrently with the specification as a text file via EFS-Web, in compliance with the American Standard Code for Information Interchange (ASCII), with a file name of 464173seqlist.txt, a creation date of Jul. 30, 2015 and a size of 139 KB. The sequence listing filed via EFS-Web is part of the specification and is hereby incorporated in its entirety by reference herein.

### BACKGROUND OF THE INVENTION

Interleukin-2 (IL-2) is a biologic that has been used in attempts to boost immune responses in cancer and HIV/AID patients. More recently lower doses of IL-2 have been used to selectively boost tolerance to suppress unwanted immune responses associated with autoimmune-like attack of self tissues. Importantly, these low doses of IL-2 have not shown any signs of enhancing or re-activation of autoreactive T cells. Nevertheless, IL-2 has important drawbacks as a therapeutic, including a very short-half life in vivo, which limits its efficacy, and toxicity at high doses. For these reasons new IL-2 biologics are needed having improved pharmacokinetics and durability of responses for use.

### SUMMARY OF THE INVENTION

Various methods and compositions are provided which can be employed to modulate the immune system. Compositions include a fusion protein comprising: (a) a first polypeptide comprising Interleukin-2 (IL-2) or a functional variant or fragment thereof; and (b) a second polypeptide, fused in frame to the first polypeptide, wherein the second polypeptide comprises an extracellular domain of Interleukin-2 Receptor alpha (IL-2Rα) or a functional variant or fragment thereof, and wherein the fusion protein has IL-2 activity.

Various methods are provided for decreasing the immune response in a subject comprising administering to a subject in need of a decrease in the immune response a therapeutically effective amount of the IL-2/IL-2Rα fusion protein disclosed herein.

Further provided are methods for increasing the immune response in a subject comprising administering to a subject in need of an increase in the immune response a therapeutically effective amount of the IL-2/IL-2Rα fusion protein disclosed herein. Further provided are methods for increasing T regulatory cell activity.

Additional methods including enhancing the immunogenicity of a vaccine or overcoming a suppressed immune response to a vaccine in a subject, comprising: (a) administering to the subject a therapeutically effective amount of the IL-2/IL-2Rα fusion protein disclosed herein; and, (b) administering to the subject a vaccine, wherein the fusion protein enhances the immunogenicity of the vaccine or overcomes the suppressed immune response to the vaccine.

### BRIEF DESCRIPTION OF THE DRAWINGS

FIG. 1 provides a schematic of an IL-2/IL-2Rα fusion protein, where L=leader peptide, LK=linker region, G=glycine, H=histidine, and T=termination codon.

FIG. 2A and FIG. 2B provide the deduced protein sequences of non-limiting examples of IL-2/IL-2Rα fusion proteins. FIG. 2A provides the deduced protein sequences of non-limiting examples of mouse IL-2/IL-2Rα fusion proteins. The sequences of mouse IL-2 and IL-2Rα are shown above and below, respectively, of the fusion proteins. The sequence denoted as IL-2 is set forth in SEQ ID NO: 3; the sequence denoted as IL-2-(G4S)4-IL-2Rα is set forth in SEQ ID NO: 54; the sequence denoted as IL-2-(G4S)5-IL-2Rα is set forth in SEQ ID NO:55; the sequence denoted as IL-2-(G3S)4-IL-2Rα is set forth in SEQ ID NO:56; the sequence denoted as IL-2-(G3S)3-IL-2Rα is set forth in SEQ ID NO: 57; and the extracellular domain of IL-2Rα is set forth in SEQ ID NO: 10. FIG. 2B provides the deduced protein sequences of non-limiting examples of human IL-2/IL-2Rα fusion proteins. The sequences of human IL-2 and IL-2Rα are shown above and below, respectively, of the fusion proteins. The sequence denoted as IL-2 is set forth in SEQ ID NO: 1; the sequence denoted as IL-2-(G3S)2-IL-2Rα is set forth in SEQ ID NO: 58; the sequence denoted as IL-2-(G3S)3-IL-2Rα is set forth in SEQ ID NO:59; the sequence denoted as IL-2-(G3S)4-IL-2Rα is set forth in SEQ ID NO:60; the sequence denoted as IL-2-(G4S)4-IL-2Rα is set forth in SEQ ID NO: 61; and the extracellular domain of IL-2Rα is set forth in SEQ ID NO: 7.

FIG. 3 shows the bioactivity of IL-2/IL-2Rα fusion proteins. COS-7 cells were transfected with the IL-2/IL-2Rα fusion cDNAs with the indicated linkers. Supernatants from these cells were cultured with anti-CD3 activated T cell blasts to assess IL-2 activity. (A) Proliferative responses by the T blasts after dilutions of the indicated fusion proteins. (B) Effect of anti-IL-2 on proliferation stimulated by a 1:2 dilution of the culture supernatant containing the indicated fusion proteins.

FIG. 4 shows the activity of purified IL-2/IL-2Rα fusion proteins. Supernatants of transfected CHO cells were used to purify IL-2/(G₃S)₃/IL-2Rα and IL-2/(Gly₄Ser)₄/IL-2Rα by Nickel-based affinity chromatography to the 6x-His tag. (A) IL-2 bioactivity measure by proliferation of anti-CD3 T cell blast to the indicated purified fusion protein. (B) The effect of each purified fusion protein to inhibit the binding of PC61 and 7D4 anti-IL-2Rα monoclonal antibodies, directed to non-ligand binding site, to anti-CD3 activated T cell blasts.

FIG. 5 shows that a monoclonal anti-IL-2Rα antibody that is directed to the IL-2 binding site of IL-2Rα cannot bind to the IL-2/IL-2Rα fusion protein. Purified fusion proteins with variable linkers, as indicated, were first incubated with the 3C7 anti-IL-2Rα monoclonal antibody, directed to the ligand binding site of IL-2Rα or the 7D4 monoclonal antibody, directed to a non-ligand binding site of IL-2Rα. The capacity of 3C7 or 7D4 to then bind to cell surface IL-2Rα was assessed using IL-2Rα-transfected EL4 cells.

FIG. 6 shows the biochemical properties of purified IL-2/IL-2Rα. (A) Purified IL-2/IL-2Rα was subjected to SDS-PAGE under reducing and non-reducing conditions; IL-2/IL-2Rα was visualized by Western blot analysis by probing with an antibody directed to 6x-His tag of the fusion protein. (B) The indicated amount of purified IL-2/(Gly₃Ser)₃/IL-2Rα was subjected to SDS-PAGE under reducing conditions followed by Coomassie Blue staining.

FIG. 7 shows the effect of IL-2/IL-2Rα fusion protein on IL-2-dependent signal transduction in vivo. C57BL/6 mice received a single injection i.p. of IL-2/(G₃S)₃/IL-2Rα (4000 units of IL-2 activity) and pSTAT5 levels in the indicated spleen cell populations were immediately assess. pSTAT5 levels were determined 0.5 hr after injection of the IL-2/IL-2Rα fusion protein. For CD4⁺ T cells, the cells were gated to excluded Foxp3⁺ Treg cells

FIG. 8 shows the effect of IL-2/IL-2Rα fusion protein on Tregs cells in vivo. NOD mice were injected i.p. 3 times (day 1, 3, 5) with the indicated amount of IL-2 activity associated with IL-2/(G₃S)₃/IL-2Rα. The effect on Tregs was assessed for the spleen, pancreatic lymph nodes (PLN) and pancreas 24 hr after the last injection. Evaluated were the proportion of Tregs in CD4⁺ T cells; the mean fluorescent intensity (MFI) for CD25 expression by Tregs after normalization to CD25 expression by Tregs from control treated mice; the proliferative status of Tregs as assessed by expression of the proliferative marker Ki67; and the % of Tregs that expressed Klrg1, which marks an IL-2-dependent terminally differentiated subpopulation.

FIG. 9 shows the comparison of IL-2/IL-2Rα fusion protein and recombinant IL-2 to induce changes in Treg cells in vivo. C57BL/6 mice were injected i.p. 3 times (day 1, 3, 5) with IL-2/(G₃S)₃/IL-2Rα (2000 Units), recombinant human IL-2 (25,000 Units) or preformed complexes of anti-IL-2 (Jes-6.1; 5 μg) and mouse IL-2 (10,000 Units) (IL2/IC). The effect on Tregs was assessed for the spleen 24, 72 hr and 1 week after the last injection. Treg were evaluated as described in FIG. 8.

FIG. 10 shows Limited application of low-dose IL-2 delays diabetes in NOD mice. NOD mice (8 mice/group) received IL-2/IL-2Rα, soluble IL-2Rα, or PBS according to the schedule in (A). Urine and blood glucose levels were monitored until mice reached 40 weeks of age. Mice were considered diabetic after 2 consecutive readings of glucose levels >250 mg/dl.

FIG. 11 demonstrates high-dose IL-2/IL-2Rα enhances the development of CD8⁺ T cell memory. C57BL/6 mice received congenic class I-restricted ovalbumin (OVA)-specific OT-I T cell receptor transgenic T cells. These mice were immunized and treated with a single application of IL-2/(G₃S)₃/IL-2Rα fusion protein, IL2/IC containing 15,000 units of IL-2, or recombinant IL-2 (25,000 Units). At the indicated times, the relative proportion of OT-I T cells within the total CD8⁺ T cell compartment in peripheral blood was assessed.

FIG. 12 shows the type of persistent OT-I memory cells supported by high-dose IL-2/IL-2Rα fusion protein: (A) Gating strategy to identify effector-memory (EM) and central memory (CM) cells. (B) Distribution of OT-1 memory cells 28 and 202 day post immunization for mice that also received IL-2/IL-2Rα (12,000 units).

FIG. 13 shows characterization of human IL-2/IL-2Rα fusion proteins containing glycine/serine linkers of variable length, as shown. (A) IL-2-bioactivity of purified human IL-2//IL-2Rα using the CTLL bioassay. (B) Western blot analysis of human IL-2/IL-2Rα fusion proteins after SDS-PAGE under reducing conditions.

FIG. 14 shows human IL-2/IL-2Rα fusion protein bind monoclonal anti-IL-2Rα antibodies. Purified fusion proteins with the indicated linkers were first incubated with the BC96 anti-IL-2Rα monoclonal antibody, directed to the ligand binding region of human IL-2Rα or the M-A257 monoclonal antibody, directed to a non-ligand binding region of human IL-2Rα. The capacity of BC96 or M-A257 to then bind to cell surface IL-2Rα was assessed using IL-2Rα-transfected CHO cells.

FIG. 15 shows IL-2 interacts with the IL-2 binding site of IL-2Rα in the context of human IL-2/IL-2Rα fusion proteins. IL-2-bioactivity of the indicated fusion proteins with variable glycine/serine linkers was assessed using CTLL cells. Mut refers to fusion proteins where IL-2Rα contained Arg³⁵→Thr, Arg³⁶→Ser mutations. Western blot analysis confirmed similar amounts of all fusion proteins (not shown).

### DETAILED DESCRIPTION OF THE INVENTION

The present inventions now will be described more fully hereinafter with reference to the accompanying drawings, in which some, but not all embodiments of the invention are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

### I. Overview

Current technology relies on the use of recombinant Interleukin-2 (IL-2), which has poor pharmacological properties, especially a short half-life that limits its usefulness. Provided herein are Interleukin-2/Interleukin-2 receptor alpha (IL-2/IL-2Rα) fusion proteins have intrinsic properties that separate them from recombinant IL-2 and other IL-2 fusion proteins. First, the size of the IL-2/IL-2Rα fusion protein will increase its half-life in vivo. Second, the weak interaction between IL-2 and IL-2Rα (one subunit of the IL-2R) in the context of the IL-2/IL-2Rα fusion protein provides another mechanism to prolong the availability of the IL-2. While not being limited to the specific mechanism of action, the prolonged availability of the IL-2 activity might occur through a competitive interaction between the IL-2 moiety with IL-2Rα of the IL-2/IL-2Rα fusion and with cells that express the IL-2R.

### II. Interleukin-2/Interleukin-2 Receptor Alpha Fusion Proteins and Polynucleotides Encoding the Same

A fusion protein is provided which comprises a first polypeptide comprising interleukin-2 (IL-2) or a functional variant or fragment thereof fused in frame to a second polypeptide comprising or consisting of the extracellular domain of the Interleukin-2 Receptor Alpha (IL-2Rα) polypeptide or a functional variant or fragment thereof.

As used herein, “fusion protein” refers to the in frame genetic linkage of at least two heterologous polypeptides. Upon transcription/translation, a single protein is made. In this way, multiple proteins, or fragments thereof can be incorporated into a single polypeptide. “Operably linked” is intended to mean a functional linkage between two or more elements. For example, an operable linkage between two polypeptides fuses both polypeptides together in frame to produce a single polypeptide fusion protein. In a particular aspect, the fusion protein further comprises a third polypeptide which, as discussed in further detail below, can comprise a linker sequence.

The IL-2/IL-2Rα fusion protein or the active variant or fragment thereof can have one or more the following properties/activities: (1) increasing activity of regulatory T cells (Tregs) and/or increasing immune tolerance in low dose IL-2 based therapies; (2) increasing immune response and memory in higher dose therapies; (3) increasing IL-2 availability when compared to recombinant IL-2; and/or (4) increasing persistent IL-2 stimulation of IL-2R bearing lymphocytes in vivo. Such activity and methods of assaying are disclosed in further detail elsewhere herein. See, for example, Example 1 provided herein.

In one non-limiting embodiment, an increased activity of Tregs that results from the IL-2/IL-2Rα fusion protein or the active variant or fragment thereof can be assayed in a variety of ways including, for example, (1) an increased representation and number of Tregs in the CD4⁺ T cell compartment; (2) upregulation of IL-2-dependent CD25; (3) increased proliferation as assessed by expression of the proliferative marker Ki67; and (4) an increased fraction of IL-2-dependent terminally differentiated KIrg1⁺ Treg subset. Such effects on Tregs can be seen in, for example, in the spleen and the inflamed pancreas.

In one non-limiting embodiment, the IL-2/IL-2Rα fusion protein or the active variant or fragment thereof increases tolerogenic and immune suppressive Tregs and immunity through increasing T effector/memory responses and, in further embodiments, it exhibits improved pharmacokinetics by delivering such responses at (1) lower effective levels of IL-2 activity compared to native or recombinant IL-2; (2) displays more persistent biological responses than native or recombinant IL-2; and/or (3) retains the hierarchy with Tregs responsive at lower level doses that T effector/memory cells.

In specific embodiments, the fusion protein has an improved activity over the native or recombinant IL-2. For example, the effect of the IL-2/IL-2Rα fusion protein can increase tolerogenic Tregs at about 2 fold, 5 fold, 10 fold, 20 fold, 30 fold, 40 fold, 50 fold, 60 fold, 70 fold, 80 fold, 90 fold, 100 fold 150 fold, 200 fold or lower level IL-2 activity in comparison to native or recombinant IL-2. In other embodiments, the IL-2/IL-2Rα fusion protein is more effective than native or recombinant IL-2 in inducing persistent augmentation of Tregs and related properties.

Various IL-2 and IL-2Rα fragments and variants from a variety of organism can be used to generate the IL-2/IL-2Rα extracellular domain fusion proteins provided herein. Such components are discussed in further detailed elsewhere herein. Examples of non-limiting unprocessed IL-2/IL-2Rα extracellular domain fusion proteins are set forth in SEQ ID NO: 17, 19, 21, 23, 25, 27, 29, 36, 38, 44, 46, 54, 55, 56, 58, 59, 60, 61, 62, and 64, while non-limiting examples of mature forms of the IL-2/IL-Rα extracellular domain fusion proteins are set forth in SEQ ID NOS: 16, 18, 20, 22, 24, 26, 37, 39, 43, 45, and 57. Non-limiting examples of polynucleotides encoding such fusion proteins are set forth in SEQ ID NO:29, 30, 31, 32, 33, 34, 42, 47, 48, 49, 63, and 65.

The term “secretory signal sequence” denotes a polynucleotide sequence that encodes a polypeptide (a “secretory peptide”) that, as a component of a larger polypeptide, directs the larger polypeptide through a secretory pathway of the cell in which it is synthesized. The larger polypeptide is commonly cleaved to remove the secretory peptide during the transit through the secretory pathway. As used herein, a “mature” form of a fusion protein or polypeptide comprises the processed form of the polypeptide that has had the secretory peptide removed. As used herein, the “unprocessed” form of the fusion protein retains the secretory peptide sequence.

Biologically active fragments and variants of the mature and unprocessed form of the IL-2/IL-Rα extracellular domain fusion proteins, and the polynucleotide encoding the same, are also provided. Such a functional polypeptide fragment can comprise at least 5, 6, 7, 8, 9, 10, 11, 12, 13, 14, 15, 16, 17, 18, 19, 20, 30, 40, 50, 75, 100, 150, 200, 250, 300, 350, 400, 450, 500 or more continuous amino acids of any one of SEQ ID NO: 16, 17, 18, 19, 20, 21, 22, 23, 24, 25, 26, 27, 36, 37, 38, 39, 43, 44, 45, 46, 54, 55, 56, 57, 58, 59, 60, 61, 62, or 64. Alternatively, a functional polypeptide variant can comprise at least 75%, 80%, 85%, 90%, 91%, 92%, 93%, 94%, 95%, 96%, 97%, 98%, or 99% sequence identity to the sequence set forth in SEQ ID NO: 16, 17, 18, 19, 20, 21, 22, 23, 24, 25, 26, 27, 36, 37, 38, 39, 43, 44, 45, 46, 54, 55, 56, 57, 58, 59, 60, 61, 62, or 64.

Active variants and fragments of polynucleotides encoding the IL-2/IL-Rα extracellular domain fusion proteins are further provided. Such polynucleotide can comprise at least 100, 200, 300, 400, 500, 600, 700, 800, 1000, 1100, 1200, 1300, 1500, 1800, 2000 continuous nucleotides of SEQ ID NO: 29, 30, 31, 32, 33, 34, 42, 47, 48, 49, 63 or 65 or the polynucleotide encoding the polypeptides set forth in SEQ ID NO: 16, 17, 18, 19, 20, 21, 22, 23, 24, 25, 26, 27, 36, 37, 38, 39, 43, 44, 45, 46, 54, 55, 56, 57, 58, 59, 60, 61, 62, or 64 and continue to encode a functional IL-2/IL-Rα extracellular domain fusion protein. Alternatively, a functional polynucleotide can comprise at least 75%, 80%, 85%, 90%, 91%, 92%, 93%, 94%, 95%, 96%, 97%, 98%, or 99% sequence identity to the sequence set forth in SEQ ID NO: 29, 30, 31, 32, 33, 34, 42, 47, 48, 49, 63 or 65 or the polynuclotide encoding the polypeptides set forth in SEQ ID NO: 16, 17, 18, 19, 20, 21, 22, 23, 24, 25, 26, 27, 36, 37, 38, 39, 43, 44, 45, 46, 54, 55, 56, 57, 58, 59, 60, 61, 62, or 64 and continue to encode a functional IL-2/IL-Rα extracellular domain fusion proteins.

It is further recognized that the components of the IL-2/IL-2Rα fusion protein can be found any order. In one embodiment, the IL-2 polypeptide is at the N-terminus and the extracellular domain of IL-2Rα is at the C-terminus of the fusion protein.

i. Interleukin-2

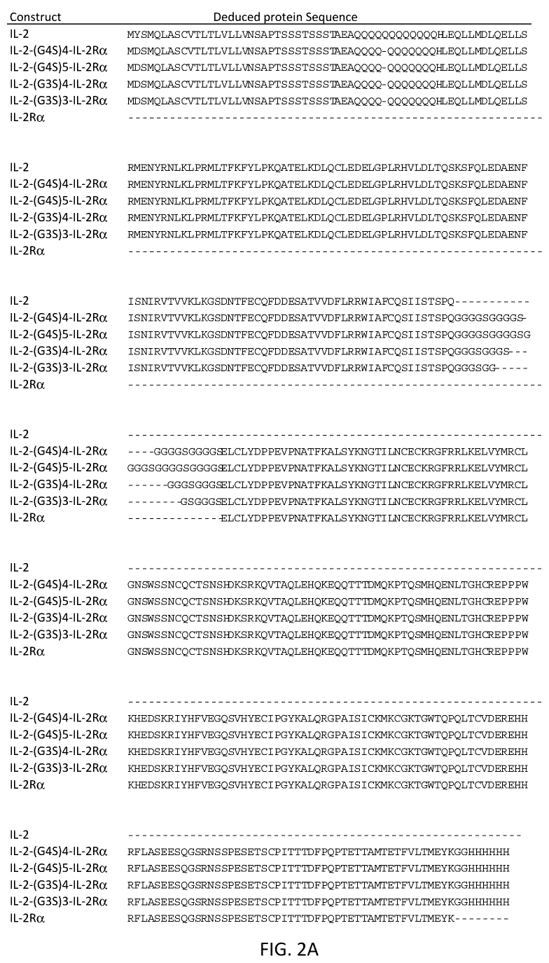

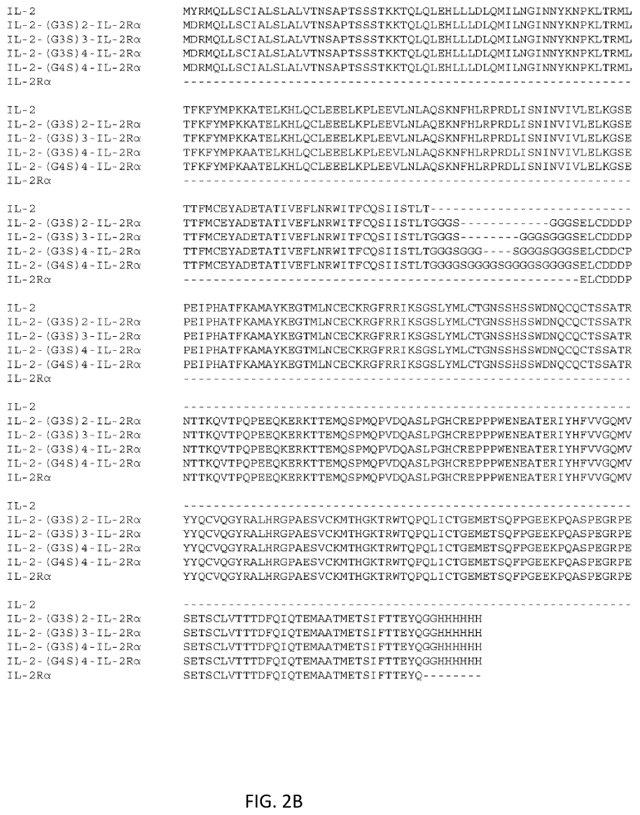

As used herein, “Interleukin-2” or “IL-2” refers to any native or recombinant IL-2 from any vertebrate source, including mammals such as primates (e.g. humans) and rodents (e.g. mice and rats), and domesticated or agricultural mammals unless otherwise indicated. The term encompasses unprocessed IL-2, as well as, any form of IL-2 that results from processing in the cell (i.e, the mature form of IL-2). The term also encompasses naturally occurring variants and fragments of IL-2, e.g. splice variants or allelic variants, and non-naturally occurring variants. The amino acid sequence of an exemplary mature form of human IL-2 (having the 20 amino acid signal sequence) is shown in SEQ ID NO: 2. Unprocessed human IL-2 additionally comprises an N-terminal 20 amino acid signal peptide (SEQ ID NO: 1), which is absent in the mature IL-2 molecule. The amino acid sequence of an exemplary mature form of mouse IL-2 (having the 20 amino acid signal sequence) is shown in SEQ ID NO: 4. Unprocessed mouse IL-2 additionally comprises an N-terminal 20 amino acid signal peptide (SEQ ID NO: 3), which is absent in the mature IL-2 molecule. See also FIG. 2A and FIG. 2B. By a “native IL-2”, also termed “wild-type IL-2”, is meant a naturally occurring or recombinant IL-2.

Additional nucleic acid and amino acid sequences for IL-2 are known. Sec, for example, GenBank Accession Nos: Q7JFM2 (Aotus lemurinus (Gray-bellied night monkey)); Q7JFM5 (Aotus nancymaae (Ma's night monkey)); P05016 (Bos taurus (Bovine)); Q29416 (Canis familiaris (Dog) (Canis lupus familiaris)); P36835 (Capra hircus (Goat)); and, P37997 (Equus caballus (Horse)).

Biologically active fragments and variants of IL-2 are also provided. Such IL-2 active variants or fragments will retain IL-2 activity. The phrase “biological activity of IL-2” refers to one or more of the biological activities of IL-2, including but not limited to, the ability to stimulate IL-2 receptor bearing lymphocytes. Such activity can be measured both in vitro and in vivo. IL-2 is a global regulator of immune activity and the effects seen here are the sum of such activities. For example, it is regulates survival activity (Bcl-2), induces T effector activity (IFN-gamma, Granzyme B, and Perforin), and promotes T regulatory activity (FoxP3). Sec, for example, Malek et al. (2010) Immunity 33 (2): 153-65, herein incorporated by reference in its entirety.

Biologically active variants of IL-2 are known. Sec, for example, US Application Publications 20060269515 and 20060160187 and WO 99/60128, each of which is herein incorporated by reference.

Biologically active fragments and variants of IL-2 can be employed in the fusion proteins disclosed herein. Such a functional fragment can comprise at least 10, 11, 12, 13, 14, 15, 16, 17, 18, 19, 20, 30, 40, 50, 75, 100, 125, 150 or more continuous amino acids of SEQ ID NO: 1, 2, 3, or 4. Alternatively, a functional variant can comprise at least 75%, 80%, 85%, 90%, 91%, 92%, 93%, 94%, 95%, 96%, 97%, 98%, or 99% sequence identity to the sequence set forth in SEQ ID NO: 1, 2, 3, or 4.

Active variants and fragments of polynucleotides encoding the IL-2 proteins are further provided. Such polynucleotide can comprise at least 100, 200, 300, 400, 500, 600, 700 continuous nucleotides of polypeptide encoding SEQ ID NO: 1, 2, 3, or 4, and continue to encode a protein having IL-2 activity. Alternatively, a functional polynucleotide can comprise at least 75%, 80%, 85%, 90%, 91%, 92%, 93%, 94%, 95%, 96%, 97%, 98%, or 99% sequence identity to the polypeptide encoding the amino sequence set forth in SEQ ID NO: 1, 2, 3, or 4 and continue to encode a functional IL-2 polypeptide.

ii. Interleukin-2 Receptor Alpha

The term “CD25” or “IL-2 receptor α” or “IL-2Rα” as used herein, refers to any native or recombinant IL-2Rα from any vertebrate source, including mammals such as primates (e.g. humans) and rodents (e.g., mice and rats) and domesticated or agricultural mammals unless otherwise indicated. The term also encompasses naturally occurring variants of IL-2Rα, e.g. splice variants or allelic variants, or non-naturally occurring variants. Human IL-2 exerts its biological effects via signaling through its receptor system, IL-2R. IL-2 and its receptor (IL-2R) are required for T-cell proliferation and other fundamental functions which are crucial of the immune response. IL-2R consists of 3 noncovalently linked type I transmembrane proteins which are the alpha (p55), beta (p75), and gamma (p65) chains. The human IL-2R alpha chain contains an extracellular domain of 219 amino acids, a transmembrane domain of 19 amino acids, and an intracellular domain of 13 amino acids. The secreted extracellular domain of IL-2R alpha (IL-2R-a) can be employed in the fusion proteins describe herein.

The amino acid sequence of an exemplary mature form of human IL-2Rα is shown in SEQ ID NO: 6. Unprocessed human IL-2Rα is shown in SEQ ID NO: 5. The extracellular domain of SEQ ID NO: 6 is set forth in SEQ ID NO: 7. The amino acid sequence of an exemplary mature form of mouse IL-2Rα is shown in SEQ ID NO: 9. Unprocessed mouse IL-2Rα is shown in SEQ ID NO: 8. The extracellular domain of SEQ ID NO: 9 is set forth in SEQ ID NO: 10. By a “native IL-2Rα”, also termed “wild-type IL-2Rα”, is meant a naturally occurring or recombinant IL-2Rα. The sequence of a native human IL-2Rα molecule is shown in SEQ ID NO: 5 and 6.

Nucleic acid and amino acid sequences for IL-2Rα are known. See, for example, GenBank Accession Nos: NP_001030597.1 (P. troglodytes); NP_001028089.1 (M. mulatta); NM_001003211.1 (C. lupus); NP_776783.1 (B. taurus); NP_032393.3 (M. musculus); and, NP_037295.1 (R. norvegicus), each of which is herein incorporated by reference.

Biologically active fragments and variants of the extracellular domain of IL-2Rα are also provided. Such IL-2Rα extracellular domain active variants or fragments will retain the IL-2Rα extracellular domain activity. The phrase “biological activity of the IL-2Rα extracellular domain” refers to one or more of the biological activities of extracellular domain of IL-2Rα, including but not limited to, the ability to enhance intracellular signaling in IL-2 receptor responsive cells. Non-limiting examples of biologically active fragments and variants of the IL-2Rα are disclosed, for example, in Robb et al., Proc. Natl. Acad. Sci. USA, 85:5654-5658, 1988, which is herein incorporated by reference.

Biologically active fragments and variants of the extracellular domain of IL-2Rα can be employed in the fusion proteins disclosed herein. Such a functional fragment can comprise at least 10, 11, 12, 13, 14, 15, 16, 17, 18, 19, 20, 30, 40, 50, 75, 100, 125, 150, 175, 200, 215 or greater continuous amino acids of the extracellular domain of any one of SEQ ID NO: 6, 9, 7, 10, 5, or 8. Alternatively, a functional variant can comprise at least 75%, 80%, 85%, 90%, 91%, 92%, 93%, 94%, 95%, 96%, 97%, 98%, or 99% sequence identity to the sequence set forth in SEQ ID NO: 6, 9, 7, 10, 5, or 8.

In one embodiment, the fusion proteins provided herein can comprise at least one mutation within the extracellular domain of IL-2Rα. In a specific embodiment, the Arginine at position 35 of IL-2Rα can be mutated to a Threonine and/or the Arginine at position 36 of IL-2Rα can be mutated to a Serine. Such a fusion protein can have increased IL-2 activity compared to a fusion protein not comprising these mutations in the extracellular domain of IL-2Rα and/or compared to native or recombinant IL-2. The amino acid sequences of exemplary fusion proteins comprising IL-2Rα with mutations within the extracellular domain of IL-2Rα are set forth in SEQ ID NOS: 62 and 64. In one embodiment, the fusion protein comprises the amino acid sequence of any one of SEQ ID NO: 62 or 64; or a sequence having at least 80%, 85%, 90%, or 95% to any one of SEQ ID NO: 62 or 64.

Active variants and fragments of polynucleotides encoding the extracellular domain of IL-2Rα are further provided. Such polynucleotide can comprise at least 100, 200, 300, 400, 500, 600 or greater continuous nucleotides of polypeptide encoding SEQ ID NO: 6, 9, 7, 10, 5, or 8 and continue to encode a protein having the extracellular domain activity of IL-2Rα. Alternatively, a functional polynucleotide can comprise at least 80%, 85%, 90%, 91%, 92%, 93%, 94%, 95%, 96%, 97%, 98%, or 99% sequence identity to the polypeptide encoding the amino sequence set forth in SEQ ID NO: 6, 9, 7, 10, 5, or 8 and continue to encode a protein having the extracellular domain activity of IL-2Rα.

iii. Additional Components

The IL-2/IL-2Rα fusion proteins can further comprise additional elements. Such elements can aid in the expression of the fusion protein, aid in the secretion of the fusion protein, improve the stability of the fusion protein, allow for more efficient purification of the protein, and/or modulate the activity of the fusion protein.

“Heterologous” in reference to a polypeptide or polynucleotide is a polypeptide or polynucleotide that originates from a different protein or polynucleotide. The additional components of the fusion protein can originate from the same organism as the other polypeptide components of the fusion protein, or the additional components can be from a different organism than the other polypeptide components of the fusion protein.

In one embodiment, the IL-2/IL-2Rα fusion protein comprises a linker sequence located between the IL-2 polypeptide and the IL-2Rα polypeptide. The linker can be of any length and can comprise at least 1, 2, 3, 4, 5, 6, 7, 8, 9, 10, 11, 12, 13, 14, 15, 16, 17, 18, 19, 20, 25, 30, 31, 32, 33, 34, 35, 36, 37, 38, 39, 40, 50 or 60 or more amino acids. In one embodiment, the linker sequence comprises glycine amino acid residues. In other instances, the linker sequence comprises a combination of glycine and serine amino acid residues. Such glycine/serine linkers can comprises any combination of the amino acid residues, including, but not limited to, the peptide GGGS or GGGGS or repeats of the same, including 1, 2, 3, 4, 5, 6, 7, 8, 9, 10 or more repeats of these given peptides. For example, linker sequences can comprise GGGSGGGSGGGS (SEQ ID NO: 13) (also noted as (Gly₃Ser)₃); GGGSGGGSGGGSGGGS (SEQ ID NO: 11) (also noted as (Gly₃Ser)₄); or (Gly₃Ser)₅; (Gly₃Ser)₆; (Gly₃Ser)₇, etc. Linker sequences can further comprise (Gly₄Ser)₃ as set forth in SEQ ID NO: 50; GGGGSGGGGSGGGGSGGGGS (SEQ ID NO: 40) (also noted as (Gly₄Ser)₄); GGGGSGGGGSGGGGSGGGGSGGGGS (SEQ ID NO: 41) (also noted as (Gly₄Ser)₅); (Gly₄Ser)₂, (Gly₄Ser)₁, (Gly₄Ser)₆; (Gly₄Ser)₇; (Gly₄Ser)₈, etc. In addition, active variants and fragments of any linker can further be employed in the fusion protein disclosed herein.

It is further recognized that the polynucleotide encoding the IL-2/IL-2Rα fusion protein can comprise additional elements that aid in the translation of the fusion protein. Such sequences include, for example, Kozak sequences attached to the 5′ end of the polynucleotide encoding the fusion protein. The Kozak consensus sequence is a sequence which occurs on eukaryotic mRNA that plays a role in the initiation of the translation process and has the consensus (gcc)gccRccAUGG (SEQ ID NO: 35); wherein (1) a lower case letter denotes the most common base at a position where the base can nevertheless vary; (2) upper case letters indicate highly-conserved bases, i.e. the ‘AUGG’ sequence is constant or rarely, if ever, changes, with the exception being the IUPAC ambiguity code ‘R’ which indicates that a purine (adenine or guanine) is normally observed at this position; and (3) the sequence in brackets ((gcc)) is of uncertain significance. In one embodiment, the Kozak sequence comprises the sequence set forth in SEQ ID NO: 53.

In one non-limiting embodiment, the IL-2/IL-2Rα fusion protein comprises an IL-2 leader optimized Kozak sequence as set forth in SEQ ID NO: 28 or a functional variant or fragment thereof. A functional variant or fragment of a Kozak sequence will retain the ability to increase translation of the protein when compared to the level of translation from a sequence lacking the leader. Such a functional fragment can comprise at least 5, 6, 7, 8, 9, 10, 11, 12, 13, 14, 15, 16, 17, 18, 19, 20, 30, 40 continuous nucleotides of a kozak sequence or the sequence set forth in SEQ ID NO: 28 or 53. Alternatively, a functional variant can comprise at least 80%, 85%, 90%, 91%, 92%, 93%, 94%, 95%, 96%, 97%, 98%, or 99% sequence identity to the kozak sequence or the sequence set forth in SEQ ID NO: 28 or 53.

In still further embodiments, the IL-2/IL-2Rα fusion protein comprises one or more tags at the C-terminus to aid in the purification of the polypeptide. Such tags are known and include, for example, a Histidine tag. In specific embodiments a 6×His tag is employed. It is further recognized that an additional linker sequence can be employed between the fusion protein and the His tag.

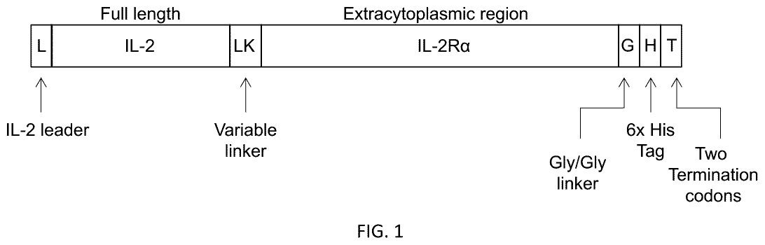

Non-limiting embodiment of an IL-2/IL-2Rα fusion protein is set forth in FIG. 1, FIG. 2A, and FIG. 2B. Such a fusion protein comprises a leader peptide, IL-2 or a functional variant or fragment thereof, a variable linker, IL-2Rα, a glycine linker, 6×his tag, and two termination codons.

iv. Variants and Fragments
a. Polynucleotides

Fragments and variants of the polynucleotides encoding the IL-2/IL-2Rα extracellular domain fusion protein or the various components contained therein (i.e., the IL-2Rα extracellular domain, the IL-2Rα polypeptides, the linker sequences and/or Kozak sequences) can be employed in the various methods and compositions of the invention. By “fragment” is intended a portion of the polynucleotide and hence the protein encoded thereby or a portion of the polypeptide. Fragments of a polynucleotide may encode protein fragments that retain the biological activity of the native protein and hence have IL-2 activity, IL-2Rα extracellular domain activity, IL-2/IL-2Rα fusion protein activity, or if encoding a linker sequence, provide for the desired activity of the IL-2/IL-2Rα fusion protein.

A biologically active portion of a IL-2Rα extracellular domain, IL-2 polypeptide, IL-2/IL-2Rα fusion protein, Kozak sequence, or linker sequence can be prepared by isolating a portion of one of the polynucleotides encoding the portion of the IL-2Rα extracellular domain or IL-2 polypeptide and expressing the encoded portion of the polypeptide (e.g., by recombinant expression in vitro), and assessing the activity of the portion of the IL-2Rα extracellular domain or/and IL-2 polypeptide or the activity of the IL-2/ILRα fusion protein.

“Variant” sequences have a high degree of sequence similarity. For polynucleotides, conservative variants include those sequences that, because of the degeneracy of the genetic code, encode the amino acid sequence of one of the IL-2Rα extracellular domain polypeptides, IL-2 polypeptides, IL-2/IL-2Rα fusion proteins, or linker sequences. Variants such as these can be identified with the use of well-known molecular biology techniques, as, for example, polymerase chain reaction (PCR) and hybridization techniques. Variant polynucleotides also include synthetically derived nucleotide sequences, such as those generated, for example, by using site-directed mutagenesis but which still encode an IL-2Rα extracellular domain, IL-2 polypeptide, IL-2/IL-2Rα fusion protein, a Kozak sequence, or the linker sequence.

b. Polypeptides

By “variant” protein is intended a protein derived from the native protein by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein. Variant proteins are biologically active, that is they continue to possess the desired biological activity, that is, IL-2/IL-2Rα fusion protein activity, IL-2 activity or IL-2Rα extracellular domain activity. Such variants may result from, for example, genetic polymorphism or from human manipulation. Biologically active variants of a IL-2/IL-2Rα fusion protein or any one of its components (i.e., an IL-2Rα extracellular domain polypeptide, a IL-2 polypeptide, or a linker sequence) will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to the amino acid sequence for the native protein as determined by sequence alignment programs and parameters described elsewhere herein. A biologically active variant of a protein may differ from that protein by as few as 1-15 amino acid residues, as few as 1-10, such as 6-10, as few as 5, as few as 4, 3, 2, or even 1 amino acid residue.

Proteins may be altered in various ways including amino acid substitutions, deletions, truncations, and insertions. Methods for such manipulations are generally known in the art. For example, amino acid sequence variants of the IL-2Rα extracellular domain, IL-2 polypeptide, IL-2/IL-2Rα fusion protein, or linker sequences can be prepared by mutations in the DNA. Methods for mutagenesis and nucleotide sequence alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, cds. (1983) Techniques in Molecular Biology (MacMillan Publishing Company, New York) and the references cited therein. Guidance as to appropriate amino acid substitutions that do not affect biological activity of the protein of interest may be found in the model of Dayhoff et al. (1978) Atlas of Protein Sequence and Structure (Natl. Biomed. Res. Found., Washington, D.C.), herein incorporated by reference. Conservative substitutions, such as exchanging one amino acid with another having similar properties, may be preferable.

Thus, the polynucleotides disclosed herein can include the naturally occurring sequences, the “native” sequences, as well as mutant forms. Likewise, the proteins used in the methods of the invention encompass naturally occurring proteins as well as variations and modified forms thereof. Such variants will continue to possess the ability to implement a recombination event. Generally, the mutations made in the polynucleotide encoding the variant polypeptide should not place the sequence out of reading frame, and/or create complementary regions that could produce secondary mRNA structure. Sec, EP Patent Application Publication No. 75,444.

Variant polynucleotides and proteins also encompass sequences and proteins derived from a mutagenic and recombinogenic procedure such as DNA shuffling. With such a procedure, one or more different IL-2Rα extracellular domain or IL-2 coding sequences can be manipulated to create a new IL-2Rα extracellular domain or IL-2 polypeptides possessing the desired properties. In this manner, libraries of recombinant polynucleotides are generated from a population of related sequence polynucleotides comprising sequence regions that have substantial sequence identity and can be homologously recombined in vitro or in vivo. Strategies for such DNA shuffling are known in the art. See, for example, Stemmer (1994) Proc. Natl. Acad. Sci. USA 91:10747-10751; Stemmer (1994) Nature 370:389-391; Crameri et al. (1997) Nature Biotech. 15:436-438; Moore et al. (1997) J. Mol. Biol. 272:336-347; Zhang et al. (1997) Proc. Natl. Acad. Sci. USA 94:4504-4509; Crameri et al. (1998) Nature 391:288-291; and U.S. Pat. Nos. 5,605,793 and 5,837,458.

### III. Polynucleotides Encoding the IL-2/IL-2Rα Fusion Proteins and Methods of Making

Compositions further include isolated polynucleotides that encode the various fusion proteins described herein above, and variants and fragments thereof. Vectors and expression cassettes comprising the polynucleotides described herein are further disclosed. Expression cassettes will generally include a promoter operably linked to a polynucleotide and a transcriptional and translational termination region.

The use of the term “polynucleotide” is not intended to limit the present invention to polynucleotides comprising DNA. Those of ordinary skill in the art will recognize that polynucleotides, can comprise ribonucleotides and combinations of ribonucleotides and deoxyribonucleotides. Such deoxyribonucleotides and ribonucleotides include both naturally occurring molecules and synthetic analogues.

An “isolated” or “purified” polynucleotide or protein, or biologically active portion thereof, is substantially or essentially free from components that normally accompany or interact with the polynucleotide or protein as found in its naturally occurring environment. Thus, an isolated or purified polynucleotide or protein is substantially free of other cellular material, or culture medium when produced by recombinant techniques, or substantially free of chemical precursors or other chemicals when chemically synthesized. Optimally, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived. For example, in various embodiments, the isolated polynucleotide can contain less than about 5 kb, 4 kb, 3 kb, 2 kb, 1 kb, 0.5 kb, or 0.1 kb of nucleotide sequence that naturally flank the polynucleotide in genomic DNA of the cell from which the polynucleotide is derived. A protein that is substantially free of cellular material includes preparations of protein having less than about 30%, 20%, 10%, 5%, or 1% (by dry weight) of contaminating protein. When the protein of the invention or biologically active portion thereof is recombinantly produced, optimally culture medium represents less than about 30%, 20%, 10%, 5%, or 1% (by dry weight) of chemical precursors or non-protein-of-interest chemicals.

Conventional molecular biology, microbiology, and recombinant DNA techniques within the skill of the art may be employed herein. Such techniques are explained fully in the literature. Sec, e.g., Sambrook et al., “Molecular Cloning: A Laboratory Manual” (1989); “Current Protocols in Molecular Biology” Volumes I-III [Ausubel, R. M., ed. (1994)]; “Cell Biology: A Laboratory Handbook” Volumes I-III [J. E. Celis, ed. (1994))]; “Current Protocols in Immunology” Volumes I-III [Coligan, J. E., cd. (1994)]; “Oligonucleotide Synthesis” (M. J. Gait ed. 1984); “Nucleic Acid Hybridization” [B. D. Hames & S. J. Higgins eds. (1985)]; “Transcription And Translation” [B. D. Hames & S. J. Higgins, eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984).

A vector which comprises the above-described polynucleotides operably linked to a promoter is also provided herein. A nucleotide sequence is “operably linked” to an expression control sequence (e.g., a promoter) when the expression control sequence controls and regulates the transcription and translation of that sequence. The term “operably linked” when referring to a nucleotide sequence includes having an appropriate start signal (e.g., ATG) in front of the nucleotide sequence to be expressed and maintaining the correct reading frame to permit expression of the sequence under the control of the expression control sequence and production of the desired product encoded by the sequence. If a gene that one desires to insert into a recombinant nucleic acid molecule does not contain an appropriate start signal, such a start signal can be inserted in front of the gene. A “vector” is a replicon, such as plasmid, phage or cosmid, to which another nucleic acid segment may be attached so as to bring about the replication of the attached segment. The promoter may be, or is identical to, a bacterial, yeast, insect or mammalian promoter. Further, the vector may be a plasmid, cosmid, yeast artificial chromosome (YAC), bacteriophage or eukaryotic viral DNA.

Other numerous vector backbones known in the art as useful for expressing protein may be employed. Such vectors include, but are not limited to: adenovirus, simian virus 40 (SV40), cytomegalovirus (CMV), mouse mammary tumor virus (MMTV), Moloney murine leukemia virus, DNA delivery systems, i.e. liposomes, and expression plasmid delivery systems. Further, one class of vectors comprises DNA elements derived from viruses such as bovine papilloma virus, polyoma virus, baculovirus, retroviruses or Semliki Forest virus. Such vectors may be obtained commercially or assembled from the sequences described by methods well-known in the art.

A host vector system for the production of a polypeptide which comprises the vector of a suitable host cell is provided herein. Suitable host cells include, but are not limited to, prokaryotic or eukaryotic cells, e.g. bacterial cells (including gram positive cells), yeast cells, fungal cells, insect cells, and animal cells. Numerous mammalian cells may be used as hosts, including, but not limited to, the mouse fibroblast cell NIH 3T3, CHO cells, HeLa cells, Ltk⁻ cells, etc. Additional animal cells, such as R1.1, B-W and L-M cells, African Green Monkey kidney cells (e.g., COS 1, COS 7, BSC1, BSC40, and BMT10), insect cells (e.g., Sf9), and human cells and plant cells in tissue culture can also be used.

A wide variety of host/expression vector combinations may be employed in expressing the polynucleotide sequences presented herein. Useful expression vectors, for example, may consist of segments of chromosomal, non-chromosomal and synthetic DNA sequences. Suitable vectors include derivatives of SV40 and known bacterial plasmids, e.g., E. coli plasmids col El, pCR1, pBR322, pMB9 and their derivatives, plasmids such as RP4; phage DNAS, e.g., the numerous derivatives of phage λ, e.g., NM989, and other phage DNA, e.g., M13 and filamentous single stranded phage DNA; yeast plasmids such as the 2u plasmid or derivatives thereof; vectors useful in eukaryotic cells, such as vectors useful in insect or mammalian cells; vectors derived from combinations of plasmids and phage DNAs, such as plasmids that have been modified to employ phage DNA or other expression control sequences; and the like.

Any of a wide variety of expression control sequences (sequences that control the expression of a nucleotide sequence operably linked to it) may be used in these vectors to express the polynucleotide sequences provided herein. Such useful expression control sequences include, for example, the early or late promoters of SV40, CMV, vaccinia, polyoma or adenovirus, the lac system, the trp system, the TAC system, the TRC system, the LTR system, the major operator and promoter regions of phage λ, the control regions of fd coat protein, the promoter for 3-phosphoglycerate kinase or other glycolytic enzymes, the promoters of acid phosphatase (e.g., Pho5), the promoters of the yeast a-mating factors, and other sequences known to control the expression of genes of prokaryotic or eukaryotic cells or their viruses, and various combinations thereof.

It will be understood that not all vectors, expression control sequences and hosts will function equally well to express the polynucleotide sequences provided herein. Neither will all hosts function equally well with the same expression system. However, one skilled in the art will be able to select the proper vectors, expression control sequences, and hosts without undue experimentation to accomplish the desired expression without departing from the scope of this invention. For example, in selecting a vector, the host must be considered because the vector must function in it. The vector's copy number, the ability to control that copy number, and the expression of any other proteins encoded by the vector, such as antibiotic markers, will also be considered.

In selecting an expression control sequence, a variety of factors will normally be considered. These include, for example, the relative strength of the system, its controllability, and its compatibility with the particular nucleotide sequence or gene to be expressed, particularly as regards potential secondary structures. Suitable unicellular hosts will be selected by consideration of, e.g., their compatibility with the chosen vector, their secretion characteristics, their ability to fold proteins correctly, and their fermentation requirements, as well as the toxicity to the host of the product encoded by the nucleotide sequences to be expressed, and the ease of purification of the expression products.

In preparing the expression cassette, the various polynucleotides may be manipulated, so as to provide for the polynucleotide sequences in the proper orientation and, as appropriate, in the proper reading frame. Toward this end, adapters or linkers may be employed to join the polynucleotides or other manipulations may be involved to provide for convenient restriction sites, removal of superfluous DNA, removal of restriction sites, or the like. For example, linkers such as two glycines may be added between polypeptides. Methionine residues encoded by atg nucleotide sequences may be added to allow initiation of gene transcription. For this purpose, in vitro mutagenesis, primer repair, restriction, annealing, resubstitutions, e.g., transitions and transversions, may be involved.

Further provided is a method of producing a polypeptide which comprises expressing a polynucleotide encoding a fusion protein disclosed herein in a host cell under suitable conditions permitting the production of the polypeptide and recovering the polypeptide so produced.

### IV. Methods of Use

Various methods are provided for modulating an immune response. As used herein, the term “modulating” includes inducing, inhibiting, potentiating, elevating, increasing, or decreasing a given activity or response.

By “subject” is intended mammals, e.g., primates, humans, agricultural and domesticated animals such as, but not limited to, dogs, cats, cattle, horses, pigs, sheep, and the like. In one embodiment, the subject undergoing treatment with the pharmaceutical formulations provided herein is a human.

A “therapeutically effective amount” of an IL-2/IL-2Rα fusion protein refers to the amount of the IL-2/IL-2Rα fusion protein sufficient to elicit a desired biological response. As will be appreciated by one of ordinary skill in the art, the absolute amount of a particular IL-2/IL-2Rα fusion protein that is effective can vary depending on such factors as the desired biological endpoint, the IL-2/IL-2Rα fusion protein to be delivered, the target cell or tissue, and the like. One of ordinary skill in the art will further understand that an effective amount can be administered in a single dose, or can be achieved by administration of multiple doses (i.e., 1, 2, 3, 4, 5, 6, 7, 8, 9, 10 or more doses).

i. Methods for Increasing an Immune Response

Various methods are provided for increasing the immune response in a subject. Such methods comprise administering to a subject in need of an increase in the immune response a therapeutically effective amount of an IL-2/IL-2Rα fusion protein. As such, in specific embodiments, transient application of higher doses of IL-2 are employed to boosted immune effector and memory responses.

It is further recognized that the various IL-2/IL-2Rα fusion protein can be used in combination with an antigen to enhance the immune response to the antigen. Thus, the IL-2/IL-2Rα fusion protein can also be used as a vaccine adjuvant especially to boost cell-mediated immune memory.

For example, the IL-2/IL-2Rα fusion protein can be used to enhance a vaccine preparation. Thus, the various IL-2/IL-2Rα fusion proteins are useful for increasing the efficacy of anti-cancer vaccines or for vaccines that are poorly immunogenic. Further provided are methods for enhancing the efficacy or immunogenicity of a vaccine in a subject, or overcoming a suppressed immune response to a vaccine in a subject, including (i) administering to the subject a therapeutically effective amount of an IL-2/IL-2Rα fusion protein and (ii) administering to the subject a vaccine.

By “vaccine” is intended a composition useful for stimulating a specific immune response (or immunogenic response) in a subject. In some embodiments, the immunogenic response is protective or provides protective immunity. For example, in the case of a disease-causing organism the vaccine enables the subject to better resist infection with or disease progression from the organism against which the vaccine is directed. Alternatively, in the case of a cancer, the vaccine strengthens the subject's natural defenses against cancers that have already developed. These types of vaccines may also prevent the further growth of existing cancers, prevent the recurrence of treated cancers, and/or eliminate cancer cells not killed by prior treatments.

Representative vaccines include, but are not limited to, vaccines against diphtheria, tetanus, pertussis, polio, measles, mumps, rubella, hepatitis B, Haemophilus influenzae type b, varicella, meningitis, human immunodeficiency virus, tuberculosis, Epstein Barr virus, malaria, hepatitis E, dengue, rotavirus, herpes, human papillomavirus, and cancers. Vaccines of interest include the two vaccines that have been licensed by the U.S. Food and Drug Administration to prevent virus infections that can lead to cancer: the hepatitis B vaccine, which prevents infection with the hepatitis B virus, an infectious agent associated with liver cancer (MMWR Morb. Mortal. Wkly. Rep. 46:107-09, 1997); and Gardasil™, which prevents infection with the two types of human papillomavirus that together cause 70 percent of cervical cancer cases worldwide (Speck and Tyring, Skin Therapy Lett. 11:1-3, 2006). Other treatment vaccines of interest include therapeutic vaccines for the treatment of cancer, cervical cancer, follicular B cell non-Hodgkin's lymphoma, kidney cancer, cutaneous melanoma, ocular melanoma, prostate cancer, and multiple myeloma.

By “enhancing the efficacy” or “enhancing the immunogenicity” with regard to a vaccine is intended improving an outcome, for example, as measured by a change in a specific value, such as an increase or a decrease in a particular parameter of an activity of a vaccine associated with protective immunity. In one embodiment, enhancement refers to at least a 5%, 10%, 25%, 50%, 100% or greater than 100% increase in a particular parameter. In another embodiment, enhancement refers to at least a 5%, 10%, 25%, 50%, 100% or greater than 100% decrease in a particular parameter. In one example, enhancement of the efficacy/immunogenicity of a vaccine refers to an increase in the ability of the vaccine to inhibit or treat disease progression, such as at least a 5%, 10%, 25%, 50%, 100%, or greater than 100% increase in the effectiveness of the vaccine for that purpose. In a further example, enhancement of the efficacy/immunogenicity of a vaccine refers to an increase in the ability of the vaccine to recruit the subject's natural defenses against cancers that have already developed, such as at least a 5%, 10%, 25%, 50%, 100%, or greater than 100% increase in the effectiveness of the vaccine for that purpose.

Similarly, by “overcoming a suppressed immune response” with regard to a vaccine is intended improving an outcome, for example, as measured by a change in a specific value, such as a return to a formerly positive value in a particular parameter of an activity of a vaccine associated with protective immunity. In one embodiment, overcoming refers to at least a 5%, 10%, 25%, 50%, 100% or greater than 100% increase in a particular parameter. In one example, overcoming a suppressed immune response to a vaccine refers to a renewed ability of the vaccine to inhibit or treat disease progression, such as at least a 5%, 10%, 25%, 50%, 100%, or greater than 100% renewal in the effectiveness of the vaccine for that purpose. In a further example, overcoming a suppressed immune response to a vaccine refers to a renewed ability of the vaccine to recruit the subject's natural defenses against cancers that have already developed, such as at least a 25%, 50%, 100%, or greater than 100% renewal in the effectiveness of the vaccine for that purpose.

By “therapeutically effective amount” is intended an amount that is useful in the treatment, prevention or diagnosis of a disease or condition. As used herein, a therapeutically effective amount of an IL-2/IL-2Rα fusion protein is an amount which, when administered to a subject, is sufficient to achieve a desired effect, such as modulating an immune response in a subject without causing a substantial cytotoxic effect in the subject. As outlined above, a therapeutically effective amount of an IL-2/IL-2Rα fusion protein can be administered to a subject to increase an immune response, enhance the immune response to an antigen, enhance the efficacy or immunogenicity of a vaccine in a subject, or to overcome a suppressed immune response to a vaccine. The effective amount of an IL-2/IL-2Rα fusion protein useful for modulating such functions will depend on the subject being treated, the severity of the affliction, and the manner of administration of the IL-2/IL-2Rα fusion protein. Exemplary doses include about 10⁴ to about 10⁷ IU of IL-2 activity per adult, about 10⁴ to 10⁵ IU of IL-2 activity per adult, about 10⁵ to about 10⁶ IU of IL-2 activity per adult, about 10⁶ to about 10⁷ IU of IL-2 activity per adult. In other instances, the therapeutically effective dose of the IL-2/IL-2Rα fusion protein is about 10⁵ IU of IL-2 activity±100-fold, is about 10⁵ IU of IL-2 activity±10-fold, about 10⁵ IU of IL-2 activity±2-fold, about 10⁵ IU of IL-2 activity±20-fold, about 10⁵ IU of IL-2 activity±30-fold, about 10⁵ IU of IL-2 activity±40-fold, about 10⁵ IU of IL-2 activity±50-fold, about 10⁵ IU of IL-2 activity±60-fold, about 10⁵ IU of IL-2 activity±70-fold, about 10⁵ IU of IL-2 activity±80-fold, or about 10⁵ IU of IL-2 activity±90-fold. In a specific non-limiting embodiment, a human IL-2 fusion protein is administered at this dosage.

In one embodiment, the reference standard for the mouse IL-2 fusion protein is the mouse IL-2 is from eBiosciences (Catalog Number: 14-8021). Briefly, the bioactivity of mouse IL-2 from eBioscience is as follows: The ED50 of this protein, as measured by CTLL-2 cell proliferation assay, is less than or equal to 175 pg/mL. This corresponds to a specific activity of greater than or equal to 5.7×10⁶ Units/mg.

In another embodiment, the reference standard for the human IL-2 fusion protein is the human IL-2 drug Aldesleukin (Proleukin). Thus, the IL-2 fusion proteins disclosed herein are directly compared to the fusion protein to the IL-2 drug that is used in low dose or high dose IL-2 therapy. IL-2 activity for mouse and human IL-2 use the same assay and their activity in units/mg are similar. With respect to the human IL-2 drug, i.e. aldesleukin (Proleukin), the standard measure of an amount IL-2 is the International Unit (IU) which technically is not a fixed amount but the amount that produces a fixed effect in a specific assay of biological activity, i.e. CTLL proliferation assay. In practice, the manufacture of IL-2 is standardized and there is a conversion between drug weight and International Units. It is 1.1 mg IL-2=18 million IU (abbreviated 18 MIU).

It is furthermore understood that appropriate doses of a functional agent depend upon the potency of the active agent with respect to the activity to be modulated. Such appropriate doses may be determined using the assays described herein. In addition, it is understood that the specific dose level for any particular animal subject will depend upon a variety of factors including the activity of the specific compound employed, the age, body weight, general health, gender, and diet of the subject, the time of administration, the route of administration, the rate of excretion, and/or any drug combination.

When administration is for the purpose of treatment, administration may be for either a prophylactic or therapeutic purpose. When provided prophylactically, the substance is provided in advance of any symptom. The prophylactic administration of the substance serves to prevent or attenuate any subsequent symptom. When provided therapeutically, the substance is provided at (or shortly after) the onset of a symptom. The therapeutic administration of the substance serves to attenuate any actual symptom.

The skilled artisan will appreciate that certain factors may influence the dosage required to effectively treat a subject, including but not limited to, the severity of the disease or disorder, previous treatments, the general health and/or age of the subject, and other diseases present. Moreover, treatment of a subject with a therapeutically effective amount of an IL-2/IL-2Rα fusion protein can include a single treatment or, preferably, can include a series of treatments. It will also be appreciated that the effective dosage of an IL-2/IL-2Rα fusion protein used for treatment may increase or decrease over the course of a particular treatment. Changes in dosage may result and become apparent from the results of diagnostic assays as described herein.

Therapeutically effective amounts of an IL-2/IL-2Rα fusion protein can be determined by animal studies. When animal assays are used, a dosage is administered to provide a target in vivo concentration similar to that which has been shown to be effective in the animal assays.

ii. Methods for Decreasing an Immune Response

Various methods are provided for decreasing the immune response in a subject. Such methods comprise administering to a subject in need of a decrease in the immune response a therapeutically effective amount of an IL-2/IL-2Rα fusion protein.

There is much interest to harness the suppressive power of Tregs to inhibit unwanted immune responses. Data in mouse and man shows that enhancing IL-2R signaling with a low dose of IL-2 selectively boosts Tregs and enhances immune tolerogenic mechanisms. IL-2/IL-2Rα fusion proteins provided herein represent a new and improved form of IL-2 that more potentially enhances Tregs. Thus, the IL-2/IL-2Rα fusion proteins can be administered to patients with autoimmune diseases, chronic graft versus host disease, transplant rejection reactions, and other conditions where the goal is to suppress self-reactivity.

For example, a therapeutically effective amount of an IL-2/IL-2Rα fusion protein that promotes immune tolerance can find use, for example, in treating a subject having an autoimmune or an inflammatory disorder, including but not limited to, graft rejections and allergies. Thus, in one embodiment, a method of treating a subject having an autoimmune or inflammatory disorder is provided. Such a method comprises administering to the subject a therapeutically effective amount of an IL-2/IL-2Rα fusion protein.

Non-limiting examples of autoimmune disorders that can be treated or prevented include type 1 diabetes, multiple sclerosis, rheumatoid arthritis, celiac disease, systemic lupus erythematous, juvenile idiopathic arthritis, Crohn's disease, ulcerative colitis or systemic sclerosis, graft versus host disease, HCV-induced vasculitis, alopecia areata or psoriasis.

Additional autoimmune diseases include those where there is already an indication that Tregs may be impaired and would benefit from IL-2-dependent boosting of Tregs. In this regard, single nucleotide polymorphisms (SNPs) in IL-2, IL-2Rα, or IL-2R13 have been associated as a genetic risk for type 1 diabetes, multiple sclerosis, rheumatoid arthritis, celiac disease, systemic lupus erythematosus, juvenile idiopathic arthritis, Crohn's disease, ulcerative colitis, and systemic sclerosis. Studies suggest that the genetic risk is related to impaired Treg numbers and/or activity. In addition, low dose IL-2 therapy has shown to benefit patients with chronic GvHD and HCV-induced vasculitis. Thus, such patients populations can also be administered a therapeutically effective amount of an IL-2/IL-2Rα fusion protein.

In other embodiments, the IL-2/IL-2Rα fusion protein can be used in combination with a therapeutic agent to reduce the immune response to the agent (i.e. protein). For example, the IL-2/IL-2Rα fusion protein can be used in combination with a therapeutic protein which must be chronically administered to a subject. Thus, in a specific embodiment, the method comprises includes administering to the subject at least one additional therapeutic agent in combination with an IL-2/IL-2Rα fusion protein. Such therapeutic agents, include but are not limited to, a cytokine, a glucocorticoid, an anthracycline (e.g., doxorubicin or epirubicin), a fluoroquinolone (e.g., ciprofloxacin), an antifolate (e.g., methotrexate), an antimetabolite (e.g., fluorouracil), a topoisomerase inhibitor (e.g., camptothecin, irinotecan or etoposide), an alkylating agent (e.g., cyclophosphamide, ifosfamide, mitolactol, or melphalan), an antiandrogen (e.g., flutamide), an antiestrogen (e.g., tamoxifen), a platinum compound (e.g., cisplatin), a vinca alkaloid (e.g., vinorelbine, vinblastine or vindesine), or mitotic inhibitor (e.g., paclitaxel or docetaxel).

Moreover, the therapeutically effective amount of the IL-2/IL-2Rα fusion protein can further be administered in combination therapies to increase Tregs and tolerance. Such combination therapies can comprises the therapeutically effective amount of the IL-2/IL-2Rα fusion protein in combination with anti-TNFα or other agents to inhibit inflammatory responses.

The therapeutically effective amount of an IL-2/IL-2Rα fusion protein useful for decreasing an immune response will depend on the subject being treated, the severity of the affliction, and the manner of administration of the IL-2/IL-2Rα fusion protein. Exemplary doses include about 10³ IU to about 10⁶ IU of IL-2 activity per adult or about 10⁴ IU to about 10⁶ IU of IL-2 activity per adult. Exemplary doses include about 10³ to about 10⁶ IU of IL-2 activity per adult, about 10³ to about 10⁴ IU of IL-2 activity per adult, about 10⁴ to about 10⁶ IU of IL-2 activity per adult, about 10⁴ to 10⁵ IU of IL-2 activity per adult, or about 10⁵ to about 10⁶ IU of IL-2 activity per adult. In other instances, the therapeutically effective dose of the IL-2/IL-2Rα fusion protein is about 10⁴ IU of IL-2 activity±100-fold, is about 10⁴ IU of IL-2 activity±10-fold, about 10⁴ IU of IL-2 activity±2-fold, about 10⁴ IU of IL-2 activity±20-fold, about 10⁴ IU of IL-2 activity±30-fold, about 10⁴ IU of IL-2 activity±40-fold, about 10⁴ IU of IL-2 activity±50-fold, about 10⁴ IU of IL-2 activity±60-fold, about 10⁴ IU of IL-2 activity±70-fold, about 10⁴ IU of IL-2 activity±80-fold, or about 10⁴ IU of IL-2 activity±90-fold. In a specific non-limiting embodiment, a human IL-2 fusion protein is administered at this dosage.

In one embodiment, the reference standard for the mouse IL-2 fusion protein is the mouse IL-2 is from eBiosciences (Catalog Number: 14-8021). Briefly, the bioactivity of mouse IL-2 from eBioscience is as follows: The ED50 of this protein, as measured by CTLL-2 cell proliferation assay, is less than or equal to 175 pg/mL. This corresponds to a specific activity of greater than or equal to 5.7×10⁶ Units/mg.

In another embodiment, the reference standard for the human IL-2 fusion protein is the human IL-2 drug Aldesleukin (Proleukin). Thus, the IL-2 fusion proteins disclosed herein are directly compared to the fusion protein to the IL-2 drug that is used in low dose or high dose IL-2 therapy. IL-2 activity for mouse and human IL-2 use the same assay and their activity in units/mg are similar. With respect to the human IL-2 drug, i.e. aldesleukin (Proleukin), the standard measure of an amount IL-2 is the International Unit (IU) which technically is not a fixed amount but the amount that produces a fixed effect in a specific assay of biological activity, i.e. CTLL proliferation assay. In practice, the manufacture of IL-2 is standardized and there is a conversion between drug weight and International Units. It is 1.1 mg IL-2=18 million IU (abbreviated 18 MIU).

It is furthermore understood that appropriate doses of a functional agent depend upon the potency of the active agent with respect to the expression or activity to be modulated. Such appropriate doses may be determined using the assays described herein. In addition, it is understood that the specific dose level for any particular animal subject will depend upon a variety of factors including the activity of the specific compound employed, the age, body weight, general health, gender, and diet of the subject, the time of administration, the route of administration, the rate of excretion, and/or any drug combination.

When administration is for the purpose of treatment, administration may be for cither a prophylactic or therapeutic purpose. When provided prophylactically, the substance is provided in advance of any symptom. The prophylactic administration of the substance serves to prevent or attenuate any subsequent symptom. When provided therapeutically, the substance is provided at (or shortly after) the onset of a symptom. The therapeutic administration of the substance serves to attenuate any actual symptom.

The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of an IL-2/IL-2Rα fusion protein can include a single treatment or, preferably, can include a series of treatments. It will also be appreciated that the effective dosage of an IL-2/IL-2Rα fusion protein used for treatment may increase or decrease over the course of a particular treatment. Changes in dosage may result and become apparent from the results of diagnostic assays as described herein.

Therapeutically effective amounts of an IL-2/IL-2Rα fusion protein can be determined by animal studies. When animal assays are used, a dosage is administered to provide a target tissue concentration similar to that which has been shown to be effective in the animal assays.

iii. Pharmaceutical Composition

The various IL-2/IL-2Rα fusion proteins disclosed herein (also referred to herein as “active compounds”) can be incorporated into pharmaceutical compositions suitable for administration. Such compositions typically comprise the fusion protein and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), and transmucosal. In addition, it may be desirable to administer a therapeutically effective amount of the pharmaceutical composition locally to an area in need of treatment. This can be achieved by, for example, local or regional infusion or perfusion during surgery, topical application, injection, catheter, suppository, or implant (for example, implants formed from porous, non-porous, or gelatinous materials, including membranes, such as sialastic membranes or fibers), and the like. In another embodiment, the therapeutically effective amount of the pharmaceutical composition is delivered in a vesicle, such as liposomes (see, e.g., Langer, Science 249:1527-33, 1990 and Treat et al., in Liposomes in the Therapy of Infectious Disease and Cancer, Lopez Berestein and Fidler (eds.), Liss, N.Y., pp. 353-65, 1989).

In yet another embodiment, the therapeutically effective amount of the pharmaceutical composition can be delivered in a controlled release system. In one example, a pump can be used (see, e.g., Langer, Science 249:1527-33, 1990; Sefton, Crit. Rev. Biomed. Eng. 14:201-40, 1987; Buchwald et al., Surgery 88:507-16, 1980; Saudek et al., N. Engl. J. Med. 321:574-79, 1989). In another example, polymeric materials can be used (see, e.g., Levy et al., Science 228:190-92, 1985; During et al., Ann. Neurol. 25:351-56, 1989; Howard et al., J. Neurosurg. 71:105-12, 1989). Other controlled release systems, such as those discussed by Langer (Science 249:1527-33, 1990), can also be used.

Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes, or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EL9 (BASF; Parsippany, NJ), or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion, and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride, in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying, which yields a powder of the active ingredient plus any additional desired ingredient from a previously sterile-filtered solution thereof.

For administration by inhalation, the compounds are delivered in the form of an aerosol spray from a pressurized container or dispenser that contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art. The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated with each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such a functional compound for the treatment of individuals.

The pharmaceutical compositions can be included in a container, pack, or dispenser together with instructions for administration.

iv. Kits

As used herein, a “kit” comprises an IL-2/IL-2Rα fusion protein for use in modulating the immune response, as described elsewhere herein. The terms “kit” and “system,” as used herein are intended to refer to at least one or more IL-2/IL-2Rα fusion protein which, in specific embodiments, are in combination with one or more other types of elements or components (e.g., other types of biochemical reagents, containers, packages, such as packaging intended for commercial sale, instructions of use, and the like).

### V. Sequence Identity

As described above, active variants and fragments of the IL-2/IL-2Rα fusion proteins or the polynucleotide encoding the same, including the various components of the IL-2/IL-2Rα fusion protein are provided. Such components include, IL-2, the extracellular domain of IL-2Rα, the linker sequences or the Kozak sequence. The activity retained by the active variant or fragment of the fusion protein or a given component of the fusion protein is discussed in further detail elsewhere herein.

Such variants can have at least 90%, 91%, 92%, 93%, 94%, 95%, 96%, 97%, 98%, or 99% sequence identity to a given reference polypeptide or polynucleotide. A fragment can comprise at least 10, 20, 30, 50, 75, 100, 200, 300, 400, 500, 600, 700, 800, 900, 1000, 1500, 2000 contiguous nucleotides of a given reference nucleotide sequence or up to the full length of a given nucleotide reference sequence; or a fragment can comprise at least 10, 20, 30, 40, 50, 60, 70, 75, 80, 90, 100, 110, 120, 130, 140, 150, 160, 170, 180, 190, 200 contiguous amino acids or up to the full length of a given reference polypeptide sequence.

As used herein, “sequence identity” or “identity” in the context of two polynucleotides or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, California).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

Unless otherwise stated, sequence identity/similarity values provided herein refer to the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof. By “equivalent program” is intended any sequence comparison program that, for any two sequences in question, generates an alignment having identical nucleotide or amino acid residue matches and an identical percent sequence identity when compared to the corresponding alignment generated by GAP Version 10.

As used herein, the singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description.

The subject matter of the present disclosure is further illustrated by the following non-limiting examples.

### EXPERIMENTAL

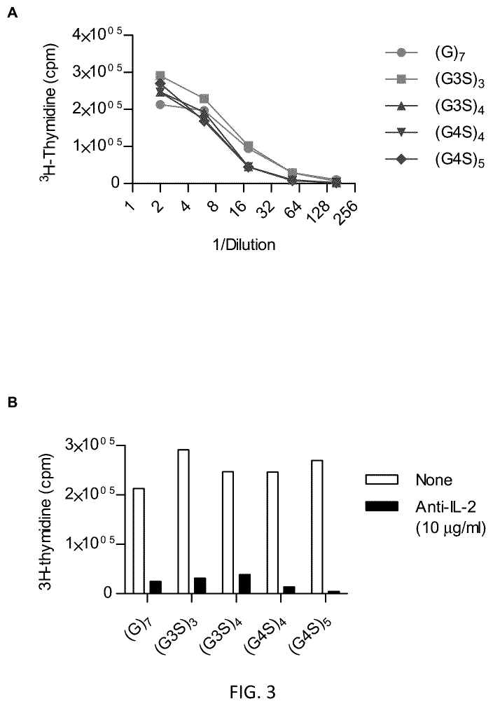

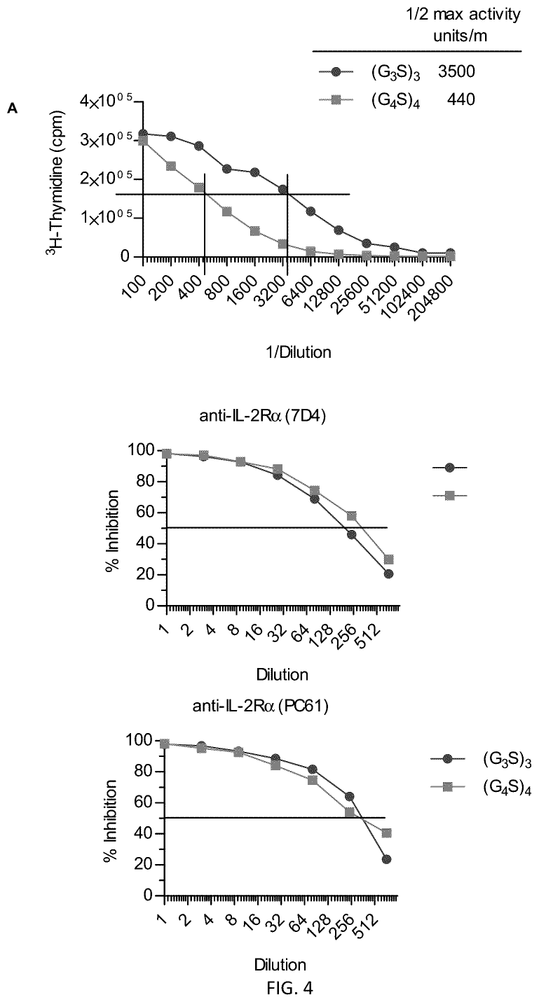

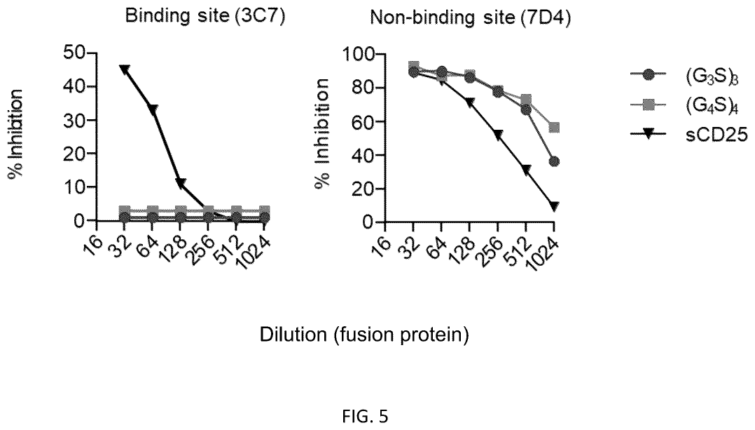

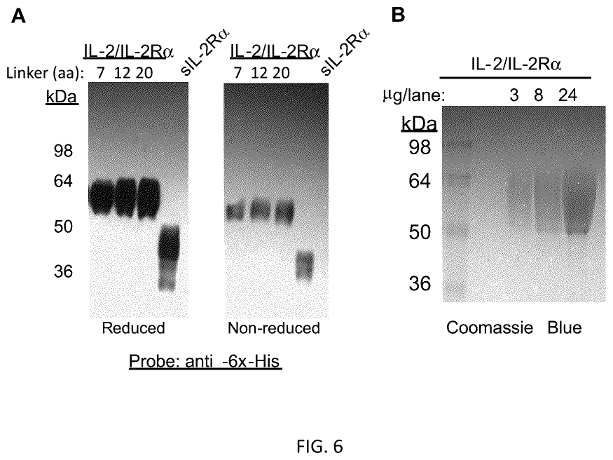

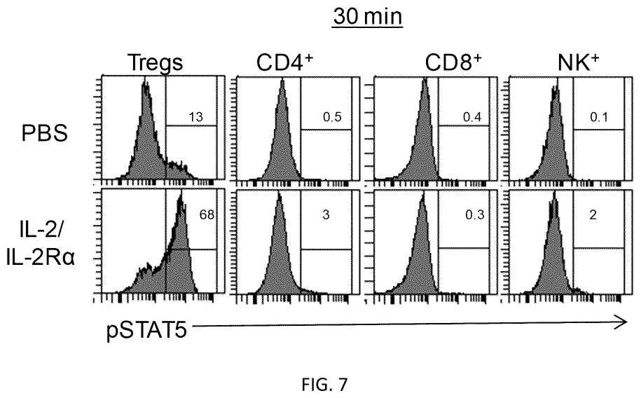

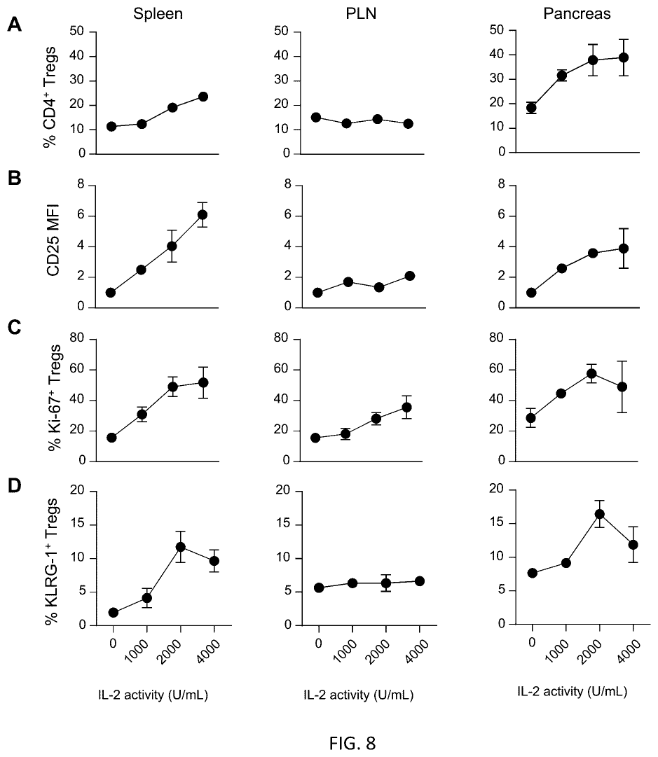

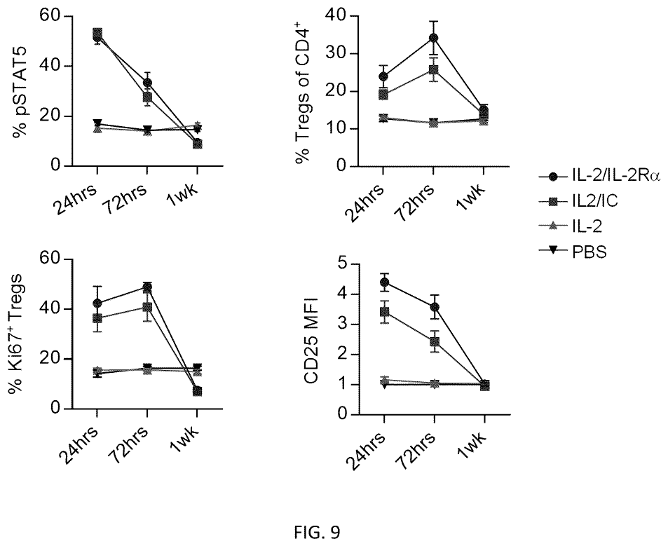

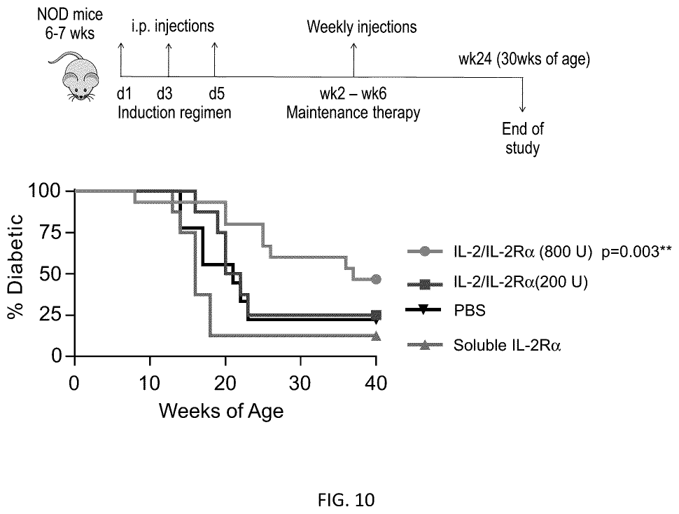

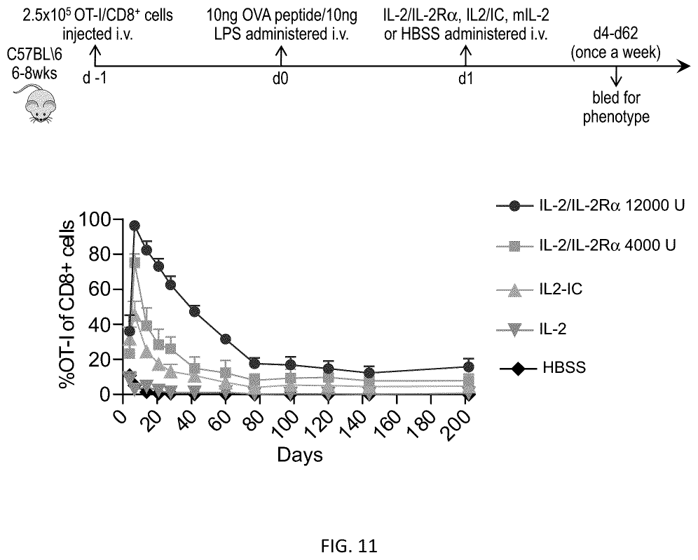

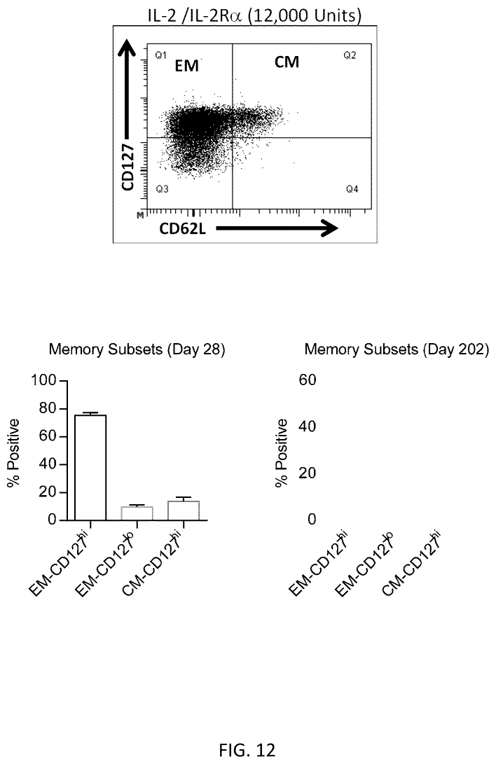

IL-2 is a biologic that has been used in attempts to boost immune responses in cancer and HIV/AID patients. More recently much lower doses of IL-2 have been used to selectively boost tolerance to suppress unwanted immune responses associated with autoimmune-like attack of self-tissues. Importantly, these low doses of IL-2 have not shown signs of enhancing or re-activation of autoreactive T cells. Nevertheless, IL-2 has important drawbacks as a therapeutic, including a very short-half life in vivo, which limits its efficacy, and toxicity at high doses. For these reasons a new IL-2 biologic has been produced with the goals of improving its pharmacokinetics and durability of responses for use 1) in low dose IL-2-based therapy to boost regulatory T cells (Tregs) and immune tolerance and 2) in adjuvant therapy with higher doses to boost immune responses and memory. To achieve these goals, IL-2/IL-2Rα fusion proteins have been developed, where these fusions were designed to increase IL-2 availability by increasing persistent IL-2 stimulation of IL-2R-bearing lymphocytes in vivo. These fusions consist of engineered proteins as follows (FIG. 1): 1) a leader sequence of IL-2 that contains an optimized Kozak sequence for efficient translation; 2) the full length sequence of IL-2; 3) a glycine or glycine/serine linker sequence of variable length; 4) the coding sequence of the expressed extracellular domain of IL-2Rα; 5) a 2 amino acid glycine spacer; 6) a six amino acid poly-histidine region for purification; and 7) two termination codons. The predicted protein sequences from these mouse and human cDNAs are shown for IL-2/(GlySer)/IL-2Rα fusion proteins in FIG. 2A and FIG. 2B, respectively. These cDNAs were cloned into the pClneo expression vector and used for expression of these fusion proteins in COS7 cells. Analysis of the culture supernatants indicated that each mouse fusion protein exhibited IL-2 bioactivity in vitro, with optimal activity associated with the IL-2/(Gly: Ser) 3/IL-2Rα fusion protein (FIG. 3A). Accordingly, inclusion of anti-IL-2 in this bioassay completely inhibited proliferation (FIG. 3B). Larger amounts of IL-2/(Gly₃Ser)₃/IL-2Rα and IL-2/(Gly₄Ser)₄/IL-2Rα were prepared after expression in CHO cells and purified by affinity chromatography through binding of the 6×His tag of the fusion protein to immobilized nickel. The mouse IL-2/(Gly₃Ser)₃/IL-2Rα fusion protein showed greater IL-2 bioactivity than IL-2/(Gly₄Ser)₄/IL-2Rα (FIG. 4A) even though both fusion proteins similarly inhibited the binding of two anti-IL2Rα antibodies (PC61 and 7D4) (FIG. 4B) to cells expressing IL-2Rα, confirming greater IL-2 activity is associated with the former fusion protein. The inhibition of binding of PC61 and 7D4 also indicates that IL-2Rα portion of the fusion protein retained sufficient tertiary structure to bind these antibodies. However, these fusion proteins did not inhibit the binding of a monoclonal antibody (3C7) directed to the IL-2 binding site of IL-2Rα, to cells expressing IL-2Rα. This result implies that IL-2 within the IL-2/IL-2Rα fusion protein in spatially near the binding site of IL-2Rα (FIG. 5). Western blot analysis of these fusion proteins showed that IL-2/IL-2Rα was 55-65 kDa, with somewhat faster mobility under non-reducing condition, and that it was approximately 15 kDa larger than that observed for soluble IL-2Rα (FIG. 6A). Correspondingly, direct analysis of the purified mouse IL-2/(Gly₃Ser)₃/IL-2Rα by SDS-PAGE was consistent with a heterogeneous 55-65 kDa monomer protein (FIG. 6B), which is the expected size for an IL-2 (15 kDa) and IL-2Rα (40-50 kDa) fusion molecule (FIG. 6), where IL-2Rα shows size heterogeneity due to extensive variable glycosylation (Malek and Korty, J. Immunol. 136:4092-4098, 1986). An immediate consequence of IL-2-dependent signal transduction is tyrosine phosphorylation of STAT5 (pSTAT5). Treatment of mice with mouse IL-2/(Gly₃Ser)₃/IL-2Rα resulted in extensive and selective activation of pSTAT5 in Tregs 30 min post-treatment (FIG. 7). Dose-response studies showed that mouse IL-2/(Gly₃Ser)₃/IL-2Rα affected a number of key activities of Tregs in vivo (FIG. 8). These effects on Tregs included: increased representation (FIG. 8A) and number (not shown) of Tregs in the CD4 T cell compartment; upregulation of IL-2-dependent CD25 (FIG. 8B); increased proliferation as assess by expression of the proliferative marker Ki67 (FIG. 8C); and increased fraction of IL-2-dependent terminally differentiated Klrg1⁺ Treg subset (FIG. 8D). These effects were most striking for Tregs in the spleen and the inflamed pancreas of non-obese diabetic (NOD) mice. 1000 units of IL-2 activity, as measured in the standard CTLL IL-2 bioassay, associated with IL-2/(Gly₃Ser)₃/IL-2Rα showed lower, but readily measurable effects on Tregs (FIG. 8). C57/BL6 mice treated with IL-2/(Gly₃Ser)₃/IL-2Rα (2000 units of IL-2 activity) were compared to mice that received recombinant IL-2 (25,000 units) or agonist complexes of IL-2/anti-IL-2 (IL2/IC) (10,000 units of IL-2 activity) (FIG. 9). IL-2/(Gly₃Ser)₃/IL-2Rα was much more effective than recombinant IL-2 and slightly more effective than IL2/IC in inducing persistent augmentation of Tregs and related properties (FIG. 9). These increases in tolerogenic Tregs occurred at 5- and 12.5-fold lower levels of IL-2 activity in comparison to IL2/IC and recombinant IL-2, respectively. When considering IL-2-dependent activation of pSTAT5 in Tregs directly ex vivo (FIG. 9), these data suggest a biological half-life of approximately 72 hours for IL-2/IL-2Rα. Pre-diabetic NOD mice underwent a short course of treatment with low amounts of IL-2/IL-2Rα (FIG. 10). A delay in the onset of diabetes was observed in those mice that were treated with 800 U of IL-2 activity associated with IL-2/IL-2Rα. With respect to immunity, application of a single high dose of IL-2/(Gly₃Ser)₃/IL-2Rα (12,000 U of IL-2 activity) also substantially boosted CD8⁺ T cell responses, especially long-lived memory cells (FIG. 11). Early after immunization (day 28), CD44^{hi} CD62L^{lo} CD127^{hi} effector-memory (EM) cells dominated the memory pool; however, with increasing time CD44^{hi} CD62L^{hi} CD127^{hi} central memory (CM) cells increased, and CM cells dominated the memory pool 202 days post-immunization (FIG. 12). Thus, IL-2/(Gly₃Ser)₃/IL-2Rα functions in an analogous manner to recombinant IL-2 to boost tolerogenic and immune suppressive Tregs and immunity through increasing T effector/memory responses, but it exhibits improved pharmacokinetics by delivering such responses: 1) at a lower effective levels of IL-2 activity; 2) with more persistent biological responses; and 3) retaining the hierarchy with Tregs responsive at lower doses than T effector/memory cells. These findings support the notion that IL-2/IL-2Rα fusion proteins represent an improved and new class of drugs to deliver IL-2 activity to selectively boost immune tolerance or immune memory when administered at the proper dose and regimen.

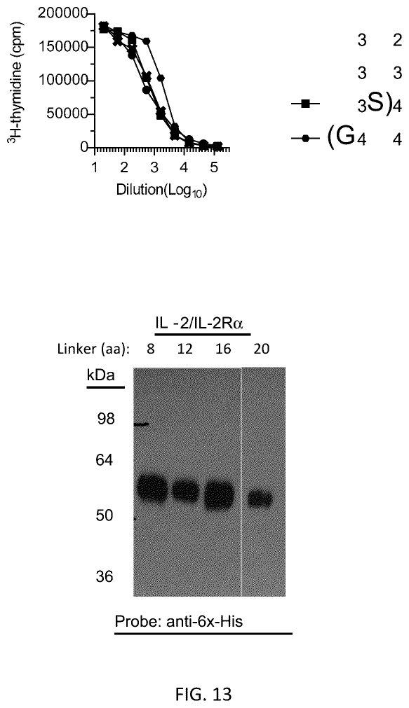

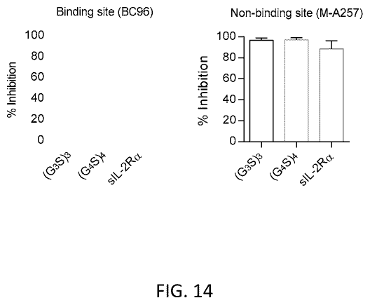

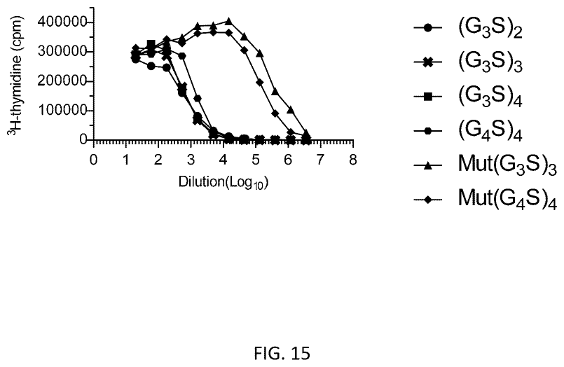

IL-2/IL-2Rα fusion proteins were also produced that comprise human IL-2 and human IL-2Rα (FIG. 1, FIG. 2B). These cDNAs were expressed in CHO cells and the secreted fusion proteins were purified on Nickel affinity chromatography based on the 6x-His tag. Fusion proteins varied in the length of the glycine/serine linkers in an analogous manner to those used for mouse IL-2/IL-2Rα. All 4 of the resulting human IL-2/IL-2Rα fusion proteins exhibited IL-2 bioactivity using the mouse CTLL assay (FIG. 13A). Western blot analysis confirmed that human IL-2/IL-2Rα also showed a heterogeneous band between 55-60 kDa (FIG. 13B), consistent with highly glycosylated molecules expected for IL-2 linked to IL-2Rα. The IL-2/IL-2Rα fusion proteins with the (G3S); and especially the (G₄S)₄ linkers may have greater activity because less fusion protein was seen even though equivalent amount of IL-2 activity was loaded on each lane (FIG. 13B). The capacity of the fusion protein to inhibit the binding of anti-IL-2Rα monoclonal antibodies, M-A257 and BC96, to cells bearing human IL-2Rα indicates that IL-2Rα of the fusion protein retained sufficient tertiary structure to bind these antibodies (FIG. 14). However, these fusion proteins only partially inhibited the binding of a monoclonal antibody (BC96) directed to the IL-2 binding site of IL-2Rα, implying that IL-2 within the IL-2/IL-2Rα fusion protein is spatially near the binding site of IL-2Rα. Moreover, we estimated the specific activity of the mouse and human IL-2/IL-2Rα fusion proteins containing the (G₃S)₃ linker to be 80 and 2000 pM, respectively, for 1 unit/ml of IL-2 bioactivity activity. These values are much higher than the activity of recombinant IL-2, which is 10 pM at 1 unit/ml. The distinct activities between human and mouse IL-2/IL-2Rα is at least partially accounted for by a relative ineffectiveness of the human fusion protein to support the proliferation of mouse CTLL cells in the bioassay compared to the mouse fusion proteins or mouse and human recombinant IL-2 (not shown). These relatively low specific activities and the antibody blocking results (FIG. 5 and FIG. 12) raised the possibility that there is a specific intramolecular interaction between IL-2 and IL-2Rα in the context of the fusion protein that limits the amount of IL-2 in the fusion protein to stimulate cells bearing the IL-2R. To directly test this notion, two arginine residues within the IL-2 binding site of human IL-2Rα (see Robb et al., Proc. Natl. Acad. Sci. USA, 85:5654-5658, 1988) were mutated to threonine and serine. We detected much greater bioactivity associated with these mutant IL-2R fusion proteins (FIG. 15); the specific activity of the mutated IL-2/IL-2Rα fusion proteins was estimated to be approximately 5 pM for 1 unit/ml of IL-2 activity, a value very similar to recombinant IL-2. Thus, these data indicate that human IL-2/IL-2Rα is biologically active and one specific mechanism of action that accounts for the prolonged IL-2 activity in these fusion proteins is through a competitive interaction between the IL-2 moiety with the IL-2 binding region of IL-2Rα of the fusion protein and with cells that express the IL-2R.

TABLE 1
Summary of Sequences

| SEQ | | | | |
|---|---|---|---|---|
| ID | AA/ | | | |
| NO | NT | Source | Description | |
| 1 | AA | Human | IL-2- | GenBank Acc. No. AAB46883 IL-2 |
| | | | unprocessed | myrmqllsci alslalvtns aptssstkkt qlqlehllld lqmilnginn |
| | | | | yknpkltrmltfkfympkka telkhlqcle eelkpleevl nlaqsknfhl rprdlisnin |
| | | | | vivlelkgsettfmceyade tativeflnr witfcqsiis tlt |
| 2 | AA | Human | IL-2-mature | GenBank AAB46883 with first 20 aa removed |
| | | | form | aptssstkkt qlqlehllld lqmilnginn yknpkltrmltfkfympkka telkhlqcle |
| | | | | eelkpleevl nlaqsknfhl rprdlisnin vivlelkgse |
| | | | | ttfmceyade tativeflnr witfcqsiis tlt |
| 3 | AA | Mouse | IL-2 | Acc No. P04351 |
| | | | unprocessed | MYSMQLASCV TLTLVLLVNS APTSSSTSSS TAEAQQQQQQ |
| | | | | QQQQQQHLEQ LLMDLQELLS RMENYRNLKL PRMLTFKFYL |
| | | | | PKQATELKDL QCLEDELGPL RHVLDLTQSK SFQLEDAENF |
| | | | | ISNIRVTVVK LKGSDNTFEC QFDDESATVV DFLRRWIAFC QSIISTSPQ |
| 4 | AA | Mouse | IL-2 mature | Mature form of Acc No. P04351 |
| | | | form | MYSMQLASCV TLTLVLLVNS APTSSSTSSS TAEAQQQQQQ |
| | | | | QQQQQQHLEQ LLMDLQELLS RMENYRNLKL PRMLTFKFYL |
| | | | | PKQATELKDL QCLEDELGPL RHVLDLTQSK SFQLEDAENF |
| | | | | ISNIRVTVVK LKGSDNTFEC QFDDESATVV DFLRRWIAFC QSIISTSPQ |
| 5 | AA | Human | IL-2Rα | Genebank Acc No. NP_000408.1 |
| | | | unprocessed | mdsyllmwgl ltfimvpgcq aelcdddppe iphatfkama ykegtmlnce |
| | | | form | ckrgfrriksgslymlctgn sshsswdnqc qctssatrnt tkqvtpqpee |
| | | | | qkerkttemq spmqpvdqaslpghcreppp weneateriy hfvvgqmvyy |
| | | | | qcvqgyralh rgpaesvckm thgktrwtqpqlictgemet sqfpgeekpq |
| | | | | aspegrpese tsclvtttdf qiqtemaatm etsiftteyqvavagcvfll isvlllsglt |
| | | | | wqrrqrksrr ti |
| 6 | AA | Human | IL-2Rα mature | First 1-21 AA removed from NP_000408.1 |
| | | | form | elcdddppe iphatfkama ykegtmlnce ckrgfrriksgslymlctgn |
| | | | | sshsswdnqc qctssatrnt tkqvtpqpee qkerkttemq spmqpvdqas |
| | | | | lpghcreppp weneateriy hfvvgqmvyy qcvqgyralh rgpaesvckm |
| | | | | thgktrwtqpqlictgemet sqfpgeekpq aspegrpese tsclvtttdf |
| | | | | qiqtemaatm etsiftteyqvavagcvfll isvlllsglt wqrrqrksrr ti |
| 7 | AA | Human | Mature form of | ELCDDDPPEIPHATFKAMAYKEGTMLNCECKRGFRRIKSGSLYMLCTGN |
| | | | IL-2Rα | SSHSSWDNQCQCTSSATRNTTKQVTPQPEEQKERKTTEMQSPMQPVD |
| | | | extracellular | QASLPGHCREPPPWENEATERIYHFVVGQMVYYQCVQGYRALHRGPA |
| | | | domain | ESVCKMTHGKTRWTQPQLICTGEMETSQFPGEEKPQASPEGRPESETS |
| | | | | CLVTTTDFQIQTEMAATMETSIFTTEYQ |
| 8 | AA | Mouse | IL-2Rα | Acc No. NP_032393.3 |
| | | | unprocessed | meprllmlgf lsltivpscr aelclydppe vpnatfkals ykngtilnce |
| | | | form | ckrgfrrlkelvymrclgns wssncqctsn shdksrkqvt aqlehqkeqq |
| | | | | tttdmqkptq smhqenltghcrepppwkhe dskriyhfve gqsvhyecip |
| | | | | gykalqrgpa isickmkcgk tgwtqpqltcvderehhrfl aseesqgsrn |
| | | | | sspesetscp itttdfpqpt ettamtetfv ltmeykvavasclfllisil llsgltwqhr |
| | | | | wrksrrti |
| 9 | AA | Mouse | IL-2Rα mature | aa 1-21 removed from Acc No. NP_032393.3 |
| | | | form | elclydppe vpnatfkals ykngtilnce ckrgfrrlke lvymrclgns wssncqctsn |
| | | | | shdksrkqvt aqlehqkeqq tttdmqkptq smhqenltgh crepppwkhe |
| | | | | dskriyhfve gqsvhyecip gykalqrgpa isickmkcgk tgwtqpqltc |
| | | | | vderehhrfl aseesqgsrn sspesetscp itttdfpqpt ettamtetfv |
| | | | | ltmeykvava sclfllisil llsgltwqhr wrksrrti |
| 10 | AA | Mouse | Mature form of | elclydppe vpnatfkals ykngtilnce ckrgfrrlke lvymrclgns wssncqctsn |
| | | | IL-2Rα | shdksrkqvt aqlehqkeqq tttdmqkptq smhqenltgh crepppwkhe |
| | | | extracellular | dskriyhfve gqsvhyecip gykalqrgpa isickmkcgk tgwtqpqltc |
| | | | domain | vderehhrfl aseesqgsrn sspesetscp itttdfpqpt ettamtetfv ltmeyk |
| 11 | AA | | (Gly3Ser)4 | GGGSGGGSGGGSGGGS |
| | | | linker | |
| 12 | AA | | (Gly3Ser)2 | GGGSGGGS |
| | | | linker | |
| 13 | AA | | (Gly3Ser)3 | GGGSGGGSGGGS |
| | | | linker | |
| 14 | AA | | (Gly3Ser)5 | GGGSGGGSGGGSGGGSGGGS |
| 15 | AA | | Gly3 linker | GGG |
| 16 | AA | Mouse | Mature form of | APTSSSTSSSTAEAQQQQQQQQQQQQHLEQLLMDLQELLSRMENYRN |
| | | | IL-2 | LKLPRMLTFKFYLPKQATELKDLQCLEDELGPLRHVLDLTQSKSFQLEDAE |
| | | | (Gly4Ser)4- | NFISNIRVTVVKLKGSDNTFECQFDDESATVVDFLRRWIAFCQSIISTSPQ |
| | | | extracellular | GGGGSGGGGSGGGGSGGGGSELCLYDPPEVPNATFKALSYKNGTILNC |
| | | | domain of IL-2 | ECKRGFRRLKELVYMRCLGNSWSSNCQCTSNSHDKSRKQVTAQLEHQK |
| | | | Rα | EQQTTTDMQKPTQSMHQENLTGHCREPPPWKHEDSKRIYHFVEGQSV |
| | | | | HYECIPGYKALQRGPAISICKMKCGKTGWTQPQLTCVDEREHHRFLASE |
| | | | | ESQGSRNSSPESETSCPITTTDFPQPTETTAMTETFVLTMEYK |
| 17 | AA | Mouse | Unprocessed | MDSMQLASCVTLTLVLLVNSAPTSSSTSSSTAEAQQQQQQQQQQQQH |
| | | | form of IL-2 | LEQLLMDLQELLSRMENYRNLKLPRMLTFKFYLPKQATELKDLQCLEDEL |
| | | | (Gly4Ser)4- | GPLRHVLDLTQSKSFQLEDAENFISNIRVTVVKLKGSDNTFECQFDDESA |
| | | | extracellular | TVVDFLRRWIAFCQSIISTSPQGGGGSGGGGSGGGGSGGGGSELCLYDP |
| | | | domain of IL-2 | PEVPNATFKALSYKNGTILNCECKRGFRRLKELVYMRCLGNSWSSNCQC |
| | | | Rα | TSNSHDKSRKQVTAQLEHQKEQQTTTDMQKPTQSMHQENLTGHCRE |
| | | | | PPPWKHEDSKRIYHFVEGQSVHYECIPGYKALQRGPAISICKMKCGKTG |
| | | | | WTQPQLTCVDEREHHRFLASEESQGSRNSSPESETSCPITTTDFPQPTET |
| | | | | TAMTETFVLTMEYK |
| 18 | AA | Mouse | Mature form of | APTSSSTSSSTAEAQQQQQQQQQQQQHLEQLLMDLQELLSRMENYRN |
| | | | IL-2 | LKLPRMLTFKFYLPKQATELKDLQCLEDELGPLRHVLDLTQSKSFQLEDAE |
| | | | (Gly4Ser)5- | NFISNIRVTVVKLKGSDNTFECQFDDESATVVDFLRRWIAFCQSIISTSPQ |
| | | | extracellular | GGGGSGGGGSGGGGSGGGGSGGGGSELCLYDPPEVPNATFKALSYKN |
| | | | domain of IL-2 | GTILNCECKRGFRRLKELVYMRCLGNSWSSNCQCTSNSHDKSRKQVTA |
| | | | Rα | QLEHQKEQQTTTDMQKPTQSMHQENLTGHCREPPPWKHEDSKRIYHF |
| | | | | VEGQSVHYECIPGYKALQRGPAISICKMKCGKTGWTQPQLTCVDEREH |
| | | | | HRFLASEESQGSRNSSPESETSCPITTTDFPQPTETTAMTETFVLTMEYK |
| 19 | AA | Mouse | Unprocessed | MDSMQLASCVTLTLVLLVNSAPTSSSTSSSTAEAQQQQQQQQQQQQH |
| | | | form of IL-2 | LEQLLMDLQELLSRMENYRNLKLPRMLTFKFYLPKQATELKDLQCLEDEL |
| | | | (Gly4Ser)5- | GPLRHVLDLTQSKSFQLEDAENFISNIRVTVVKLKGSDNTFECQFDDESA |
| | | | extracellular | TVVDFLRRWIAFCQSIISTSPQGGGGSGGGGSGGGGSGGGGSGGGGSE |
| | | | domain of IL-2 | LCLYDPPEVPNATFKALSYKNGTILNCECKRGFRRLKELVYMRCLGNSWS |
| | | | Rα | SNCQCTSNSHDKSRKQVTAQLEHQKEQQTTTDMQKPTQSMHQENLT |
| | | | | GHCREPPPWKHEDSKRIYHFVEGQSVHYECIPGYKALQRGPAISICKMKC |
| | | | | GKTGWTQPQLTCVDEREHHRFLASEESQGSRNSSPESETSCPITTTDFPQ |
| | | | | PTETTAMTETFVLTMEYK |
| 20 | AA | Mouse | Mature form of | APTSSSTSSSTAEAQQQQQQQQQQQQHLEQLLMDLQELLSRMENYRN |
| | | | IL-2 | LKLPRMLTFKFYLPKQATELKDLQCLEDELGPLRHVLDLTQSKSFQLEDAE |
| | | | (Gly3Ser)4- | NFISNIRVTVVKLKGSDNTFECQFDDESATVVDFLRRWIAFCQSIISTSPQ |
| | | | extracellular | GGGSGGGSGGGSGGGSELCLYDPPEVPNATFKALSYKNGTILNCECKRG |
| | | | domain of IL-2 | FRRLKELVYMRCLGNSWSSNCQCTSNSHDKSRKQVTAQLEHQKEQQTT |
| | | | Rα | TDMQKPTQSMHQENLTGHCREPPPWKHEDSKRIYHFVEGQSVHYECIP |
| | | | | GYKALQRGPAISICKMKCGKTGWTQPQLTCVDEREHHRFLASEESQGSR |
| | | | | NSSPESETSCPITTTDFPQPTETTAMTETFVLTMEYK |
| 21 | AA | Mouse | Unprocessed | MDSMQLASCVTLTLVLLVNSAPTSSSTSSSTAEAQQQQQQQQQQQQH |
| | | | form of IL-2 | LEQLLMDLQELLSRMENYRNLKLPRMLTFKFYLPKQATELKDLQCLEDEL |
| | | | (Gly3Ser)4- | GPLRHVLDLTQSKSFQLEDAENFISNIRVTVVKLKGSDNTFECQFDDESA |
| | | | extracellular | TVVDFLRRWIAFCQSIISTSPQGGGSGGGSGGGSGGGSELCLYDPPEVP |
| | | | domain of IL-2 | NATFKALSYKNGTILNCECKRGFRRLKELVYMRCLGNSWSSNCQCTSNS |
| | | | Rα | HDKSRKQVTAQLEHQKEQQTTTDMQKPTQSMHQENLTGHCREPPPW |
| | | | | KHEDSKRIYHFVEGQSVHYECIPGYKALQRGPAISICKMKCGKTGWTQP |
| | | | | QLTCVDEREHHRFLASEESQGSRNSSPESETSCPITTTDFPQPTETTAMTE |
| | | | | TFVLTMEYK |
| 22 | AA | human | Mature form | APTSSSTKKTQLQLEHLLLDLQMILNGINNYKNPKLTRMLTFKFYMPKKA |
| | | | IL-2 | TELKHLQCLEEELKPLEEVLNLAQSKNFHLRPRDLISNINVIVLELKGSETTF |
| | | | (Gly4Ser)4- | MCEYADETATIVEFLNRWITFCQSIISTLTGGGGSGGGGSGGGGSGGGG |
| | | | extracellular | SELCDDDPPEIPHATFKAMAYKEGTMLNCECKRGFRRIKSGSLYMLCTG |
| | | | domain of IL-2 | NSSHSSWDNQCQCTSSATRNTTKQVTPQPEEQKERKTTEMQSPMQPV |
| | | | Rα | DQASLPGHCREPPPWENEATERIYHFVVGQMVYYQCVQGYRALHRGP |
| | | | | AESVCKMTHGKTRWTQPQLICTGEMETSQFPGEEKPQASPEGRPESET |
| | | | | SCLVTTTDFQIQTEMAATMETSIFTTEYQ |
| 23 | AA | Human | Unprocessed | MDRMQLLSCIALSLALVTNSAPTSSSTKKTQLQLEHLLLDLQMILNGINN |
| | | | form IL-2 | YKNPKLTRMLTFKFYMPKKATELKHLQCLEEELKPLEEVLNLAQSKNFHL |
| | | | (Gly4Ser)4- | RPRDLISNINVIVLELKGSETTFMCEYADETATIVEFLNRWITFCQSIISTLT |
| | | | extracellular | GGGGSGGGGSGGGGSGGGGSELCDDDPPEIPHATFKAMAYKEGTML |
| | | | domain of IL-2 | NCECKRGFRRIKSGSLYMLCTGNSSHSSWDNQCQCTSSATRNTTKQVT |
| | | | Rα | PQPEEQKERKTTEMQSPMQPVDQASLPGHCREPPPWENEATERIYHFV |
| | | | | VGQMVYYQCVQGYRALHRGPAESVCKMTHGKTRWTQPQLICTGEME |
| | | | | TSQFPGEEKPQASPEGRPESETSCLVTTTDFQIQTEMAATMETSIFTTEY |
| | | | | Q |
| 24 | AA | Human | Mature form | APTSSSTKKTQLQLEHLLLDLQMILNGINNYKNPKLTRMLTFKFYMPKKA |
| | | | IL-2 | TELKHLQCLEEELKPLEEVLNLAQSKNFHLRPRDLISNINVIVLELKGSETTF |
| | | | (Gly3Ser)4- | MCEYADETATIVEFLNRWITFCQSIISTLTGGGSGGGSGGGSGGGSELCD |
| | | | extracellular | DDPPEIPHATFKAMAYKEGTMLNCECKRGFRRIKSGSLYMLCTGNSSHS |
| | | | domain of IL-2 | SWDNQCQCTSSATRNTTKQVTPQPEEQKERKTTEMQSPMQPVDQAS |
| | | | Rα | LPGHCREPPPWENEATERIYHFVVGQMVYYQCVQGYRALHRGPAESVC |
| | | | | KMTHGKTRWTQPQLICTGEMETSQFPGEEKPQASPEGRPESETSCLVTT |
| | | | | TDFQIQTEMAATMETSIFTTEYQ |
| 25 | AA | Human | Unprocessed | MDRMQLLSCIALSLALVTNSAPTSSSTKKTQLQLEHLLLDLQMILNGINN |
| | | | form IL-2 | YKNPKLTRMLTFKFYMPKKATELKHLQCLEEELKPLEEVLNLAQSKNFHL |
| | | | (Gly3Ser)4- | RPRDLISNINVIVLELKGSETTFMCEYADETATIVEFLNRWITFCQSIISTLT |
| | | | extracellular | GGGSGGGSGGGSGGGSELCDDDPPEIPHATFKAMAYKEGTMLNCECK |
| | | | domain of IL-2 | RGFRRIKSGSLYMLCTGNSSHSSWDNQCQCTSSATRNTTKQVTPQPEE |
| | | | Rα | QKERKTTEMQSPMQPVDQASLPGHCREPPPWENEATERIYHFVVGQ |
| | | | | MVYYQCVQGYRALHRGPAESVCKMTHGKTRWTQPQLICTGEMETSQF |
| | | | | PGEEKPQASPEGRPESETSCLVTTTDFQIQTEMAATMETSIFTTEYQ |
| 26 | AA | Human | Mature form | APTSSSTKKTQLQLEHLLLDLQMILNGINNYKNPKLTRMLTFKFYMPKKA |
| | | | IL-2 | TELKHLQCLEEELKPLEEVLNLAQSKNFHLRPRDLISNINVIVLELKGSETTF |
| | | | (Gly3Ser)3- | MCEYADETATIVEFLNRWITFCQSIISTLTGGGSGGGSGGGSELCDDDPP |
| | | | extracellular | EIPHATFKAMAYKEGTMLNCECKRGFRRIKSGSLYMLCTGNSSHSSWD |
| | | | domain of IL-2 | NQCQCTSSATRNTTKQVTPQPEEQKERKTTEMQSPMQPVDQASLPGH |
| | | | Rα | CREPPPWENEATERIYHFVVGQMVYYQCVQGYRALHRGPAESVCKMT |
| | | | | HGKTRWTQPQLICTGEMETSQFPGEEKPQASPEGRPESETSCLVTTTDF |
| | | | | QIQTEMAATMETSIFTTEYQ |
| 27 | AA | Human | Unprocessed | MDRMQLLSCIALSLALVTNSAPTSSSTKKTQLQLEHLLLDLQMILNGINN |
| | | | form of IL-2 | YKNPKLTRMLTFKFYMPKKATELKHLQCLEEELKPLEEVLNLAQSKNFHL |
| | | | (Gly3Ser)3- | RPRDLISNINVIVLELKGSETTFMCEYADETATIVEFLNRWITFCQSIISTLT |
| | | | extracellular | GGGSGGGSGGGSELCDDDPPEIPHATFKAMAYKEGTMLNCECKRGFRR |
| | | | domain of IL-2 | IKSGSLYMLCTGNSSHSSWDNQCQCTSSATRNTTKQVTPQPEEQKERKT |
| | | | Rα | TEMQSPMQPVDQASLPGHCREPPPWENEATERIYHFVVGQMVYYQC |
| | | | | VQGYRALHRGPAESVCKMTHGKTRWTQPQLICTGEMETSQFPGEEKP |
| | | | | QASPEGRPESETSCLVTTTDFQIQTEMAATMETSIFTTEYQ |
| 28 | NT | human | IL-2 leader | gccaccATGGACAGGATGCAACTCCTGTCTTGCATTGCACTAAGTCTTG |
| | | | optimized | CACTTGTCACAAACAGT |
| | | | Kozak | |
| | | | sequence | |
| 29 | NT | Mouse | Unprocessed | ATGGACAGCATGCAGCTCGCATCCTGTGTCACATTGACACTTGTGCTC |
| | | | form of IL-2 | CTTGTCAACAGCGCACCCACTTCAAGCTCTACTTCAAGCTCTACAGCG |
| | | | (Gly4Ser)4- | GAAGCACAGCAGCAGCAGCAGCAGCAGCAGCAGCAGCAGCACCTGG |
| | | | extracellular | AGCAGCTGTTGATGGACCTACAGGAGCTCCTGAGCAGGATGGAGAA |
| | | | domain of IL-2 | TTACAGGAACCTGAAACTCCCCAGGATGCTCACCTTCAAATTTTACTT |
| | | | Rα | GCCCAAGCAGGCCACAGAATTGAAAGATCTTCAGTGCCTAGAAGATG |
| | | | | AACTTGGACCTCTGCGGCATGTTCTGGATTTGACTCAAAGCAAAAGC |
| | | | | TTTCAATTGGAAGATGCTGAGAATTTCATCAGCAATATCAGAGTAACT |
| | | | | GTTGTAAAACTAAAGGGCTCTGACAACACATTTGAGTGCCAATTCGA |
| | | | | TGATGAGTCAGCAACTGTGGTGGACTTTCTGAGGAGATGGATAGCCT |
| | | | | TCTGTCAAAGCATCATCTCAACAAGCCCTCAAggtggaggtggatctggtgg |
| | | | | aggtggatcaggtggaggtggatccggtggaggtggatctGAACTGTGTCTGTATG |
| | | | | ACCCACCCGAGGTCCCCAATGCCACATTCAAAGCCCTCTCCTACAAGA |
| | | | | ACGGCACCATCCTAAACTGTGAATGCAAGAGAGGTTTCCGAAGACTA |
| | | | | AAGGAATTGGTCTATATGCGTTGCTTAGGAAACTCCTGGAGCAGCAA |
| | | | | CTGCCAGTGCACCAGCAACTCCCATGACAAATCGAGAAAGCAAGTTA |
| | | | | CAGCTCAACTTGAACACCAGAAAGAGCAACAAACCACAACAGACATG |
| | | | | CAGAAGCCAACACAGTCTATGCACCAAGAGAACCTTACAGGTCACTG |
| | | | | CAGGGAGCCACCTCCTTGGAAACATGAAGATTCCAAGAGAATCTATC |
| | | | | ATTTCGTGGAAGGACAGAGTGTTCACTACGAGTGTATTCCGGGATAC |
| | | | | AAGGCTCTACAGAGAGGTCCTGCTATTAGCATCTGCAAGATGAAGTG |
| | | | | TGGGAAAACGGGGTGGACTCAGCCCCAGCTCACATGTGTAGATGAA |
| | | | | AGAGAACACCACCGATTTCTGGCTAGTGAGGAATCTCAAGGAAGCA |
| | | | | GAAATTCTTCTCCCGAGAGTGAGACTTCCTGCCCCATAACCACCACAG |
| | | | | ACTTCCCACAACCCACAGAAACAACTGCAATGACGGAGACATTTGTG |
| | | | | CTCACAATGGAGTATAAGGGTGGACATCACCATCACCATCACTAATA |
| | | | | A |
| 30 | NT | Mouse | Unprocessed | ATGGACAGCATGCAGCTCGCATCCTGTGTCACATTGACACTTGTGCTC |
| | | | form of IL-2 | CTTGTCAACAGCGCACCCACTTCAAGCTCTACTTCAAGCTCTACAGCG |
| | | | (Gly3Ser)4- | GAAGCACAGCAGCAGCAGCAGCAGCAGCAGCAGCAGCAGCACCTGG |
| | | | extracellular | AGCAGCTGTTGATGGACCTACAGGAGCTCCTGAGCAGGATGGAGAA |
| | | | domain of IL-2 | TTACAGGAACCTGAAACTCCCCAGGATGCTCACCTTCAAATTTTACTT |
| | | | Rα | GCCCAAGCAGGCCACAGAATTGAAAGATCTTCAGTGCCTAGAAGATG |
| | | | | AACTTGGACCTCTGCGGCATGTTCTGGATTTGACTCAAAGCAAAAGC |
| | | | | TTTCAATTGGAAGATGCTGAGAATTTCATCAGCAATATCAGAGTAACT |
| | | | | GTTGTAAAACTAAAGGGCTCTGACAACACATTTGAGTGCCAATTCGA |
| | | | | TGATGAGTCAGCAACTGTGGTGGACTTTCTGAGGAGATGGATAGCCT |
| | | | | TCTGTCAAAGCATCATCTCAACAAGCCCTCAAggtggaggttctggtggaggt |
| | | | | tcaggtggaggttcgggtggaggttctGAACTGTGTCTGTATGACCCACCCGAG |
| | | | | GTCCCCAATGCCACATTCAAAGCCCTCTCCTACAAGAACGGCACCATC |
| | | | | CTAAACTGTGAATGCAAGAGAGGTTTCCGAAGACTAAAGGAATTGGT |
| | | | | CTATATGCGTTGCTTAGGAAACTCCTGGAGCAGCAACTGCCAGTGCA |
| | | | | CCAGCAACTCCCATGACAAATCGAGAAAGCAAGTTACAGCTCAACTT |
| | | | | GAACACCAGAAAGAGCAACAAACCACAACAGACATGCAGAAGCCAA |
| | | | | CACAGTCTATGCACCAAGAGAACCTTACAGGTCACTGCAGGGAGCCA |
| | | | | CCTCCTTGGAAACATGAAGATTCCAAGAGAATCTATCATTTCGTGGAA |
| | | | | GGACAGAGTGTTCACTACGAGTGTATTCCGGGATACAAGGCTCTACA |
| | | | | GAGAGGTCCTGCTATTAGCATCTGCAAGATGAAGTGTGGGAAAACG |
| | | | | GGGTGGACTCAGCCCCAGCTCACATGTGTAGATGAAAGAGAACACC |
| | | | | ACCGATTTCTGGCTAGTGAGGAATCTCAAGGAAGCAGAAATTCTTCT |
| | | | | CCCGAGAGTGAGACTTCCTGCCCCATAACCACCACAGACTTCCCACAA |
| | | | | CCCACAGAAACAACTGCAATGACGGAGACATTTGTGCTCACAATGGA |
| | | | | GTATAAGGGTGGACATCACCATCACCATCACTAATAA |
| 31 | NT | Mouse | Unprocessed | ATGGACAGCATGCAGCTCGCATCCTGTGTCACATTGACACTTGTGCTC |
| | | | form of IL-2 | CTTGTCAACAGCGCACCCACTTCAAGCTCTACTTCAAGCTCTACAGCG |
| | | | (Gly4Ser)5- | GAAGCACAGCAGCAGCAGCAGCAGCAGCAGCAGCAGCAGCACCTGG |
| | | | extracellular | AGCAGCTGTTGATGGACCTACAGGAGCTCCTGAGCAGGATGGAGAA |
| | | | domain of IL-2 | TTACAGGAACCTGAAACTCCCCAGGATGCTCACCTTCAAATTTTACTT |
| | | | Rα | GCCCAAGCAGGCCACAGAATTGAAAGATCTTCAGTGCCTAGAAGATG |
| | | | | AACTTGGACCTCTGCGGCATGTTCTGGATTTGACTCAAAGCAAAAGC |
| | | | | TTTCAATTGGAAGATGCTGAGAATTTCATCAGCAATATCAGAGTAACT |
| | | | | GTTGTAAAACTAAAGGGCTCTGACAACACATTTGAGTGCCAATTCGA |
| | | | | TGATGAGTCAGCAACTGTGGTGGACTTTCTGAGGAGATGGATAGCCT |
| | | | | TCTGTCAAAGCATCATCTCAACAAGCCCTCAAggtggaggtggatcaggtgg |
| | | | | aggtggatctggtggaggtggatcaggtggaggtggatccggtggaggtggatctGAAC |
| | | | | TGTGTCTGTATGACCCACCCGAGGTCCCCAATGCCACATTCAAAGCCC |
| | | | | TCTCCTACAAGAACGGCACCATCCTAAACTGTGAATGCAAGAGAGGT |
| | | | | TTCCGAAGACTAAAGGAATTGGTCTATATGCGTTGCTTAGGAAACTC |
| | | | | CTGGAGCAGCAACTGCCAGTGCACCAGCAACTCCCATGACAAATCGA |
| | | | | GAAAGCAAGTTACAGCTCAACTTGAACACCAGAAAGAGCAACAAACC |
| | | | | ACAACAGACATGCAGAAGCCAACACAGTCTATGCACCAAGAGAACCT |
| | | | | TACAGGTCACTGCAGGGAGCCACCTCCTTGGAAACATGAAGATTCCA |
| | | | | AGAGAATCTATCATTTCGTGGAAGGACAGAGTGTTCACTACGAGTGT |
| | | | | ATTCCGGGATACAAGGCTCTACAGAGAGGTCCTGCTATTAGCATCTG |
| | | | | CAAGATGAAGTGTGGGAAAACGGGGTGGACTCAGCCCCAGCTCACA |
| | | | | TGTGTAGATGAAAGAGAACACCACCGATTTCTGGCTAGTGAGGAATC |
| | | | | TCAAGGAAGCAGAAATTCTTCTCCCGAGAGTGAGACTTCCTGCCCCA |
| | | | | TAACCACCACAGACTTCCCACAACCCACAGAAACAACTGCAATGACG |
| | | | | GAGACATTTGTGCTCACAATGGAGTATAAGGGTGGACATCACCATCA |
| | | | | CCATCACTAATAA |
| 32 | NT | Human | Unprocessed | ATGGACAGGATGCAACTCCTGTCTTGCATTGCACTAAGTCTTGCACTT |
| | | | form IL-2 | GTCACAAACAGTGCACCTACTTCAAGTTCTACAAAGAAAACACAGCT |
| | | | (Gly4Ser)4- | ACAACTGGAGCATTTACTGCTGGATTTACAGATGATTTTGAATGGAAT |
| | | | extracellular | TAATAATTACAAGAATCCCAAACTCACCAGGATGCTCACATTTAAGTT |
| | | | domain of IL-2 | TTACATGCCCAAGAAGGCCACAGAACTGAAACATCTTCAGTGTCTAG |
| | | | Rα | AAGAAGAACTCAAACCTCTGGAGGAAGTGCTAAATTTAGCTCAAAGC |
| | | | | AAAAACTTTCACTTAAGACCCAGGGACTTAATCAGCAATATCAACGTA |
| | | | | ATAGTTCTGGAACTAAAGGGATCTGAAACAACATTCATGTGTGAATA |
| | | | | TGCTGATGAGACAGCAACCATTGTAGAATTTCTGAACAGATGGATTA |
| | | | | CCTTTTGTCAAAGCATCATCTCAACACTGACTggtggaggtggatctggtgga |
| | | | | ggtggatcaggtggaggtggatccggtggaggtggatct |
| | | | | GAGCTCTGTGACGATGACCCGCCAGAGATCCCACACGCCACATTCAA |
| | | | | AGCCATGGCCTACAAGGAAGGAACCATGTTGAACTGTGAATGCAAG |
| | | | | AGAGGTTTCCGCAGAATAAAAAGCGGGTCACTCTATATGCTCTGTAC |
| | | | | AGGAAACTCTAGCCACTCGTCCTGGGACAACCAATGTCAATGCACAA |
| | | | | GCTCTGCCACTCGGAACACAACGAAACAAGTGACACCTCAACCTGAA |
| | | | | GAACAGAAAGAAAGGAAAACCACAGAAATGCAAAGTCCAATGCAGC |
| | | | | CAGTGGACCAAGCGAGCCTTCCAGGTCACTGCAGGGAACCTCCACCA |
| | | | | TGGGAAAATGAAGCCACAGAGAGAATTTATCATTTCGTGGTGGGGC |
| | | | | AGATGGTTTATTATCAGTGCGTCCAGGGATACAGGGCTCTACACAGA |
| | | | | GGTCCTGCTGAGAGCGTCTGCAAAATGACCCACGGGAAGACAAGGT |
| | | | | GGACCCAGCCCCAGCTCATATGCACAGGTGAAATGGAGACCAGTCA |
| | | | | GTTTCCAGGTGAAGAGAAGCCTCAGGCAAGCCCCGAAGGCCGTCCT |
| | | | | GAGAGTGAGACTTCCTGCCTCGTCACAACAACAGATTTTCAAATACA |
| | | | | GACAGAAATGGCTGCAACCATGGAGACGTCCATATTTACAACAGAGT |
| | | | | ACCAGGGTGGACATCACCATCACCATCACTAATAA |
| 33 | NT | Human | Unprocessed | ATGGACAGGATGCAACTCCTGTCTTGCATTGCACTAAGTCTTGCACTT |
| | | | form IL-2 | GTCACAAACAGTGCACCTACTTCAAGTTCTACAAAGAAAACACAGCT |
| | | | (Gly3Ser)4- | ACAACTGGAGCATTTACTGCTGGATTTACAGATGATTTTGAATGGAAT |
| | | | extracellular | TAATAATTACAAGAATCCCAAACTCACCAGGATGCTCACATTTAAGTT |
| | | | domain of IL-2 | TTACATGCCCAAGAAGGCCACAGAACTGAAACATCTTCAGTGTCTAG |
| | | | Rα | AAGAAGAACTCAAACCTCTGGAGGAAGTGCTAAATTTAGCTCAAAGC |
| | | | | AAAAACTTTCACTTAAGACCCAGGGACTTAATCAGCAATATCAACGTA |
| | | | | ATAGTTCTGGAACTAAAGGGATCTGAAACAACATTCATGTGTGAATA |
| | | | | TGCTGATGAGACAGCAACCATTGTAGAATTTCTGAACAGATGGATTA |
| | | | | CCTTTTGTCAAAGCATCATCTCAACACTGACTggtggaggttctggtggaggt |
| | | | | tcaggtggaggttcgggtggaggttctGAGCTCTGTGACGATGACCCGCCAGA |
| | | | | GATCCCACACGCCACATTCAAAGCCATGGCCTACAAGGAAGGAACCA |
| | | | | TGTTGAACTGTGAATGCAAGAGAGGTTTCCGCAGAATAAAAAGCGG |
| | | | | GTCACTCTATATGCTCTGTACAGGAAACTCTAGCCACTCGTCCTGGGA |
| | | | | CAACCAATGTCAATGCACAAGCTCTGCCACTCGGAACACAACGAAAC |
| | | | | AAGTGACACCTCAACCTGAAGAACAGAAAGAAAGGAAAACCACAGA |
| | | | | AATGCAAAGTCCAATGCAGCCAGTGGACCAAGCGAGCCTTCCAGGTC |
| | | | | ACTGCAGGGAACCTCCACCATGGGAAAATGAAGCCACAGAGAGAAT |
| | | | | TTATCATTTCGTGGTGGGGCAGATGGTTTATTATCAGTGCGTCCAGG |
| | | | | GATACAGGGCTCTACACAGAGGTCCTGCTGAGAGCGTCTGCAAAATG |
| | | | | ACCCACGGGAAGACAAGGTGGACCCAGCCCCAGCTCATATGCACAG |
| | | | | GTGAAATGGAGACCAGTCAGTTTCCAGGTGAAGAGAAGCCTCAGGC |
| | | | | AAGCCCCGAAGGCCGTCCTGAGAGTGAGACTTCCTGCCTCGTCACAA |
| | | | | CAACAGATTTTCAAATACAGACAGAAATGGCTGCAACCATGGAGACG |
| | | | | TCCATATTTACAACAGAGTACCAGGGTGGACATCACCATCACCATCAC |
| | | | | TAATAA |
| 34 | NT | Human | Unprocessed | ATGGACAGGATGCAACTCCTGTCTTGCATTGCACTAAGTCTTGCACTT |
| | | | form of IL-2 | GTCACAAACAGTGCACCTACTTCAAGTTCTACAAAGAAAACACAGCT |
| | | | (Gly3Ser)3- | ACAACTGGAGCATTTACTGCTGGATTTACAGATGATTTTGAATGGAAT |
| | | | extracellular | TAATAATTACAAGAATCCCAAACTCACCAGGATGCTCACATTTAAGTT |
| | | | domain of IL-2 | TTACATGCCCAAGAAGGCCACAGAACTGAAACATCTTCAGTGTCTAG |
| | | | Rα | AAGAAGAACTCAAACCTCTGGAGGAAGTGCTAAATTTAGCTCAAAGC |
| | | | | AAAAACTTTCACTTAAGACCCAGGGACTTAATCAGCAATATCAACGTA |
| | | | | ATAGTTCTGGAACTAAAGGGATCTGAAACAACATTCATGTGTGAATA |
| | | | | TGCTGATGAGACAGCAACCATTGTAGAATTTCTGAACAGATGGATTA |
| | | | | CCTTTTGTCAAAGCATCATCTCAACACTGACTggtggaggttctggtggaggt |
| | | | | tcaggtggaggttcgGAGCTCTGTGACGATGACCCGCCAGAGATCCCACA |
| | | | | CGCCACATTCAAAGCCATGGCCTACAAGGAAGGAACCATGTTGAACT |
| | | | | GTGAATGCAAGAGAGGTTTCCGCAGAATAAAAAGCGGGTCACTCTAT |
| | | | | ATGCTCTGTACAGGAAACTCTAGCCACTCGTCCTGGGACAACCAATG |
| | | | | TCAATGCACAAGCTCTGCCACTCGGAACACAACGAAACAAGTGACAC |
| | | | | CTCAACCTGAAGAACAGAAAGAAAGGAAAACCACAGAAATGCAAAG |
| | | | | TCCAATGCAGCCAGTGGACCAAGCGAGCCTTCCAGGTCACTGCAGGG |
| | | | | AACCTCCACCATGGGAAAATGAAGCCACAGAGAGAATTTATCATTTC |
| | | | | GTGGTGGGGCAGATGGTTTATTATCAGTGCGTCCAGGGATACAGGG |
| | | | | CTCTACACAGAGGTCCTGCTGAGAGCGTCTGCAAAATGACCCACGGG |
| | | | | AAGACAAGGTGGACCCAGCCCCAGCTCATATGCACAGGTGAAATGG |
| | | | | AGACCAGTCAGTTTCCAGGTGAAGAGAAGCCTCAGGCAAGCCCCGA |
| | | | | AGGCCGTCCTGAGAGTGAGACTTCCTGCCTCGTCACAACAACAGATT |
| | | | | TTCAAATACAGACAGAAATGGCTGCAACCATGGAGACGTCCATATTT |
| | | | | ACAACAGAGTACCAGGGTGGACATCACCATCACCATCACTAATAA |
| 35 | NT | | Kozak | (gcc)gccRccAUGG |
| | | | consensus | |
| 36 | AA | Mouse | unprocessed | MYSMQLASCVTLTLVLLVNSAPTSSSTSSSTAEAQQQQQQQQQQQQH |
| | | | form of IL-2 | LEQLLMDLQELLSRMENYRNLKLPRMLTFKFYLPKQATELKDLQCLEDEL |
| | | | (Gly3Ser)3- | GPLRHVLDLTQSKSFQLEDAENFISNIRVTVVKLKGSDNTFECQFDDESA |
| | | | extracellular | TVVDFLRRWIAFCQSIISTSPQGGGSGGGSGGGSELCLYDPPEVPNATFK |
| | | | domain of IL-2 | ALSYKNGTILNCECKRGFRRLKELVYMRCLGNSWSSNCQCTSNSHDKSR |
| | | | Rα | KQVTAQLEHQKEQQTTTDMQKPTQSMHQENLTGHCREPPPWKHEDS |
| | | | | KRIYHFVEGQSVHYECIPGYKALQRGPAISIC27KMKCGKTGWTQPQLTC |
| | | | | VDEREHHRFLASEESQGSRNSSPESETSCPITTTDFPQPTETTAMTETFVL |
| | | | | TMEYK |
| 37 | AA | Mouse | Mature form of | MDSMQLASCVTLTLVLLVNSAPTSSSTSSSTAEAQQQQQQQQQQQQH |
| | | | IL-2 | LEQLLMDLQELLSRMENYRNLKLPRMLTFKFYLPKQATELKDLQCLEDEL |
| | | | (Gly3Ser)3- | GPLRHVLDLTQSKSFQLEDAENFISNIRVTVVKLKGSDNTFECQFDDESA |
| | | | extracellular | TVVDFLRRWIAFCQSIISTSPQGGGSGGGSGGGSELCLYDPPEVPNATFK |
| | | | domain of IL-2 | ALSYKNGTILNCECKRGFRRLKELVYMRCLGNSWSSNCQCTSNSHDKSR |
| | | | Rα | KQVTAQLEHQKEQQTTTDMQKPTQSMHQENLTGHCREPPPWKHEDS |
| | | | | KRIYHFVEGQSVHYECIPGYKALQRGPAISICKMKCGKTGWTQPQLTCV |
| | | | | DEREHHRFLASEESQGSRNSSPESETSCPITTTDFPQPTETTAMTETFVLT |
| | | | | MEYK |
| 38 | AA | Human | unprocessed | MDRMQLLSCIALSLALVTNSAPTSSSTKKTQLQLEHLLLDLQMILNGINN |
| | | | form of IL-2 | YKNPKLTRMLTFKFYMPKKATELKHLQCLEEELKPLEEVLNLAQSKNFHL |
| | | | (Gly4Ser)5- | RPRDLISNINVIVLELKGSETTFMCEYADETATIVEFLNRWITFCQSIISTLT |
| | | | extracellular | GGGGSGGGGSGGGGSGGGGSGGGGSELCDDDPPEIPHATFKAMAYK |
| | | | domain of IL-2 | EGTMLNCECKRGFRRIKSGSLYMLCTGNSSHSSWDNQCQCTSSATRNT |
| | | | Rα | TKQVTPQPEEQKERKTTEMQSPMQPVDQASLPGHCREPPPWENEATE |
| | | | | RIYHFVVGQMVYYQCVQGYRALHRGPAESVCKMTHGKTRWTQPQLIC |
| | | | | TGEMETSQFPGEEKPQASPEGRPESETSCLVTTTDFQIQTEMAATMETS |
| | | | | IFTTEYQ |
| 39 | AA | human | Mature form of | APTSSSTKKTQLQLEHLLLDLQMILNGINNYKNPKLTRMLTFKFYMPKKA |
| | | | human IL-2 | TELKHLQCLEEELKPLEEVLNLAQSKNFHLRPRDLISNINVIVLELKGSETTF |
| | | | (Gly4Ser)5- | MCEYADETATIVEFLNRWITFCQSIISTLTGGGGSGGGGSGGGGSGGGG |
| | | | extracellular | SGGGGSELCDDDPPEIPHATFKAMAYKEGTMLNCECKRGFRRIKSGSLY |
| | | | domain of IL-2 | MLCTGNSSHSSWDNQCQCTSSATRNTTKQVTPQPEEQKERKTTEMQS |
| | | | Rα | PMQPVDQASLPGHCREPPPWENEATERIYHFVVGQMVYYQCVQGYR |
| | | | | ALHRGPAESVCKMTHGKTRWTQPQLICTGEMETSQFPGEEKPQASPEG |
| | | | | RPESETSCLVTTTDFQIQTEMAATMETSIFTTEYQ |
| 40 | AA | | Linker | GGGGSGGGGSGGGGSGGGGS |
| | | | sequence | |
| | | | (Gly4Ser)4 | |
| 41 | AA | | Linker | GGGGSGGGGSGGGGSGGGGSGGGGS |
| | | | sequence | |
| | | | (Gly4Ser)5 | |
| 42 | NT | mouse | Unprocessed | ATGGACAGCATGCAGCTCGCATCCTGTGTCACATTGACACTTGTGCTC |
| | | | form IL-2 | CTTGTCAACAGCGCACCCACTTCAAGCTCTACTTCAAGCTCTACAGCG |
| | | | (Gly3Ser)3- | GAAGCACAGCAGCAGCAGCAGCAGCAGCAGCAGCAGCAGCACCTGG |
| | | | extracellular | AGCAGCTGTTGATGGACCTACAGGAGCTCCTGAGCAGGATGGAGAA |
| | | | domain of IL-2 | TTACAGGAACCTGAAACTCCCCAGGATGCTCACCTTCAAATTTTACTT |
| | | | Rα | GCCCAAGCAGGCCACAGAATTGAAAGATCTTCAGTGCCTAGAAGATG |
| | | | | AACTTGGACCTCTGCGGCATGTTCTGGATTTGACTCAAAGCAAAAGC |
| | | | | TTTCAATTGGAAGATGCTGAGAATTTCATCAGCAATATCAGAGTAACT |
| | | | | GTTGTAAAACTAAAGGGCTCTGACAACACATTTGAGTGCCAATTCGA |
| | | | | TGATGAGTCAGCAACTGTGGTGGACTTTCTGAGGAGATGGATAGCCT |
| | | | | TCTGTCAAAGCATCATCTCAACAAGCCCTCAAGGTGGAGGTTCTGGT |
| | | | | GGAGGTTCAGGTGGAGGTTCGGAACTGTGTCTGTATGACCCACCCGA |
| | | | | GGTCCCCAATGCCACATTCAAAGCCCTCTCCTACAAGAACGGCACCAT |
| | | | | CCTAAACTGTGAATGCAAGAGAGGTTTCCGAAGACTAAAGGAATTGG |
| | | | | TCTATATGCGTTGCTTAGGAAACTCCTGGAGCAGCAACTGCCAGTGC |
| | | | | ACCAGCAACTCCCATGACAAATCGAGAAAGCAAGTTACAGCTCAACT |
| | | | | TGAACACCAGAAAGAGCAACAAACCACAACAGACATGCAGAAGCCA |
| | | | | ACACAGTCTATGCACCAAGAGAACCTTACAGGTCACTGCAGGGAGCC |
| | | | | ACCTCCTTGGAAACATGAAGATTCCAAGAGAATCTATCATTTCGTGGA |
| | | | | AGGACAGAGTGTTCACTACGAGTGTATTCCGGGATACAAGGCTCTAC |
| | | | | AGAGAGGTCCTGCTATTAGCATCTGCAAGATGAAGTGTGGGAAAAC |
| | | | | GGGGTGGACTCAGCCCCAGCTCACATGTGTAGATGAAAGAGAACAC |
| | | | | CACCGATTTCTGGCTAGTGAGGAATCTCAAGGAAGCAGAAATTCTTC |
| | | | | TCCCGAGAGTGAGACTTCCTGCCCCATAACCACCACAGACTTCCCACA |
| | | | | ACCCACAGAAACAACTGCAATGACGGAGACATTTGTGCTCACAATGG |
| | | | | AGTATAAGGGTGGACATCACCATCACCATCACTAATAA |
| 43 | AA | Human | Mature form | APTSSSTKKTQLQLEHLLLDLQMILNGINNYKNPKLTRMLTFKFYMPKKA |
| | | | IL-2 | TELKHLQCLEEELKPLEEVLNLAQSKNFHLRPRDLISNINVIVLELKGSETTF |
| | | | (Gly3Ser)2- | MCEYADETATIVEFLNRWITFCQSIISTLTGGGSGGGSELCDDDPPEIPHA |
| | | | extracellular | TFKAMAYKEGTMLNCECKRGFRRIKSGSLYMLCTGNSSHSSWDNQCQC |
| | | | domain of IL-2 | TSSATRNTTKQVTPQPEEQKERKTTEMQSPMQPVDQASLPGHCREPPP |
| | | | Rα | WENEATERIYHFVVGQMVYYQCVQGYRALHRGPAESVCKMTHGKTR |
| | | | | WTQPQLICTGEMETSQFPGEEKPQASPEGRPESETSCLVTTTDFQIQTE |
| | | | | MAATMETSIFTTEYQ |
| 44 | AA | Human | Unprocessed | MDRMQLLSCIALSLALVTNSAPTSSSTKKTQLQLEHLLLDLQMILNGINN |
| | | | form IL-2 | YKNPKLTRMLTFKFYMPKKATELKHLQCLEEELKPLEEVLNLAQSKNFHL |
| | | | (Gly3Ser)2- | RPRDLISNINVIVLELKGSETTFMCEYADETATIVEFLNRWITFCQSIISTLT |
| | | | extracellular | GGGSGGGSELCDDDPPEIPHATFKAMAYKEGTMLNCECKRGFRRIKSGS |
| | | | domain of IL-2 | LYMLCTGNSSHSSWDNQCQCTSSATRNTTKQVTPQPEEQKERKTTEM |
| | | | Rα | QSPMQPVDQASLPGHCREPPPWENEATERIYHFVVGQMVYYQCVQG |
| | | | | YRALHRGPAESVCKMTHGKTRWTQPQLICTGEMETSQFPGEEKPQASP |
| | | | | EGRPESETSCLVTTTDFQIQTEMAATMETSIFTTEYQ |
| 45 | AA | Human | Mature form | APTSSSTKKTQLQLEHLLLDLQMILNGINNYKNPKLTRMLTFKFYMPKKA |
| | | | IL-2 (Gly3)- | TELKHLQCLEEELKPLEEVLNLAQSKNFHLRPRDLISNINVIVLELKGSETTF |
| | | | extracellular | MCEYADETATIVEFLNRWITFCQSIISTLTGGGELCDDDPPEIPHATFKA |
| | | | domain of IL-2 | MAYKEGTMLNCECKRGFRRIKSGSLYMLCTGNSSHSSWDNQCQCTSSA |
| | | | Rα | TRNTTKQVTPQPEEQKERKTTEMQSPMQPVDQASLPGHCREPPPWEN |
| | | | | EATERIYHFVVGQMVYYQCVQGYRALHRGPAESVCKMTHGKTRWTQP |
| | | | | QLICTGEMETSQFPGEEKPQASPEGRPESETSCLVTTTDFQIQTEMAAT |
| | | | | METSIFTTEYQ |
| 46 | AA | Human | Unprocessed | MDRMQLLSCIALSLALVTNSAPTSSSTKKTQLQLEHLLLDLQMILNGINN |
| | | | form IL-2 | YKNPKLTRMLTFKFYMPKKATELKHLQCLEEELKPLEEVLNLAQSKNFHL |
| | | | (Gly3)- | RPRDLISNINVIVLELKGSETTFMCEYADETATIVEFLNRWITFCQSIISTLT |
| | | | extracellular | GGGELCDDDPPEIPHATFKAMAYKEGTMLNCECKRGFRRIKSGSLYMLC |
| | | | domain of IL-2 | TGNSSHSSWDNQCQCTSSATRNTTKQVTPQPEEQKERKTTEMQSPMQ |
| | | | Rα | PVDQASLPGHCREPPPWENEATERIYHFVVGQMVYYQCVQGYRALHR |
| | | | | GPAESVCKMTHGKTRWTQPQLICTGEMETSQFPGEEKPQASPEGRPES |
| | | | | ETSCLVTTTDFQIQTEMAATMETSIFTTEYQ |
| 47 | NT | Human | Unprocessed | ATGGACAGGATGCAACTCCTGTCTTGCATTGCACTAAGTCTTGCACTT |
| | | | form IL-2 | GTCACAAACAGTGCACCTACTTCAAGTTCTACAAAGAAAACACAGCT |
| | | | (Gly3Ser)2- | ACAACTGGAGCATTTACTGCTGGATTTACAGATGATTTTGAATGGAAT |
| | | | extracellular | TAATAATTACAAGAATCCCAAACTCACCAGGATGCTCACATTTAAGTT |
| | | | domain of IL-2 | TTACATGCCCAAGAAGGCCACAGAACTGAAACATCTTCAGTGTCTAG |
| | | | Rα | AAGAAGAACTCAAACCTCTGGAGGAAGTGCTAAATTTAGCTCAAAGC |
| | | | | AAAAACTTTCACTTAAGACCCAGGGACTTAATCAGCAATATCAACGTA |
| | | | | ATAGTTCTGGAACTAAAGGGATCTGAAACAACATTCATGTGTGAATA |
| | | | | TGCTGATGAGACAGCAACCATTGTAGAATTTCTGAACAGATGGATTA |
| | | | | CCTTTTGTCAAAGCATCATCTCAACACTGACTggtggaggttctggtggaggt |
| | | | | tcaGAGCTCTGTGACGATGACCCGCCAGAGATCCCACACGCCACATTC |
| | | | | AAAGCCATGGCCTACAAGGAAGGAACCATGTTGAACTGTGAATGCA |
| | | | | AGAGAGGTTTCCGCAGAATAAAAAGCGGGTCACTCTATATGCTCTGT |
| | | | | ACAGGAAACTCTAGCCACTCGTCCTGGGACAACCAATGTCAATGCAC |
| | | | | AAGCTCTGCCACTCGGAACACAACGAAACAAGTGACACCTCAACCTG |
| | | | | AAGAACAGAAAGAAAGGAAAACCACAGAAATGCAAAGTCCAATGCA |
| | | | | GCCAGTGGACCAAGCGAGCCTTCCAGGTCACTGCAGGGAACCTCCAC |
| | | | | CATGGGAAAATGAAGCCACAGAGAGAATTTATCATTTCGTGGTGGG |
| | | | | GCAGATGGTTTATTATCAGTGCGTCCAGGGATACAGGGCTCTACACA |
| | | | | GAGGTCCTGCTGAGAGCGTCTGCAAAATGACCCACGGGAAGACAAG |
| | | | | GTGGACCCAGCCCCAGCTCATATGCACAGGTGAAATGGAGACCAGTC |
| | | | | AGTTTCCAGGTGAAGAGAAGCCTCAGGCAAGCCCCGAAGGCCGTCC |
| | | | | TGAGAGTGAGACTTCCTGCCTCGTCACAACAACAGATTTTCAAATACA |
| | | | | GACAGAAATGGCTGCAACCATGGAGACGTCCATATTTACAACAGAGT |
| | | | | ACCAGGGTGGACATCACCATCACCATCACTAATAA |
| 48 | NT | Human | Unprocessed | ATGGACAGGATGCAACTCCTGTCTTGCATTGCACTAAGTCTTGCACTT |
| | | | form IL-2 | GTCACAAACAGTGCACCTACTTCAAGTTCTACAAAGAAAACACAGCT |
| | | | (Gly3)- | ACAACTGGAGCATTTACTGCTGGATTTACAGATGATTTTGAATGGAAT |
| | | | extracellular | TAATAATTACAAGAATCCCAAACTCACCAGGATGCTCACATTTAAGTT |
| | | | domain of IL-2 | TTACATGCCCAAGAAGGCCACAGAACTGAAACATCTTCAGTGTCTAG |
| | | | Rα | AAGAAGAACTCAAACCTCTGGAGGAAGTGCTAAATTTAGCTCAAAGC |
| | | | | AAAAACTTTCACTTAAGACCCAGGGACTTAATCAGCAATATCAACGTA |
| | | | | ATAGTTCTGGAACTAAAGGGATCTGAAACAACATTCATGTGTGAATA |
| | | | | TGCTGATGAGACAGCAACCATTGTAGAATTTCTGAACAGATGGATTA |
| | | | | CCTTTTGTCAAAGCATCATCTCAACACTGACTggtggaggtGAGCTCTGT |
| | | | | GACGATGACCCGCCAGAGATCCCACACGCCACATTCAAAGCCATGGC |
| | | | | CTACAAGGAAGGAACCATGTTGAACTGTGAATGCAAGAGAGGTTTCC |
| | | | | GCAGAATAAAAAGCGGGTCACTCTATATGCTCTGTACAGGAAACTCT |
| | | | | AGCCACTCGTCCTGGGACAACCAATGTCAATGCACAAGCTCTGCCAC |
| | | | | TCGGAACACAACGAAACAAGTGACACCTCAACCTGAAGAACAGAAA |
| | | | | GAAAGGAAAACCACAGAAATGCAAAGTCCAATGCAGCCAGTGGACC |
| | | | | AAGCGAGCCTTCCAGGTCACTGCAGGGAACCTCCACCATGGGAAAAT |
| | | | | GAAGCCACAGAGAGAATTTATCATTTCGTGGTGGGGCAGATGGTTTA |
| | | | | TTATCAGTGCGTCCAGGGATACAGGGCTCTACACAGAGGTCCTGCTG |
| | | | | AGAGCGTCTGCAAAATGACCCACGGGAAGACAAGGTGGACCCAGCC |
| | | | | CCAGCTCATATGCACAGGTGAAATGGAGACCAGTCAGTTTCCAGGTG |
| | | | | AAGAGAAGCCTCAGGCAAGCCCCGAAGGCCGTCCTGAGAGTGAGAC |
| | | | | TTCCTGCCTCGTCACAACAACAGATTTTCAAATACAGACAGAAATGGC |
| | | | | TGCAACCATGGAGACGTCCATATTTACAACAGAGTACCAGGGTGGAC |
| | | | | ATCACCATCACCATCACTAATAA |
| 49 | NT | Human | Unprocessed | ATGGACAGGATGCAACTCCTGTCTTGCATTGCACTAAGTCTTGCACTT |
| | | | form IL-2 | GTCACAAACAGTGCACCTACTTCAAGTTCTACAAAGAAAACACAGCT |
| | | | (Gly4Ser)5- | ACAACTGGAGCATTTACTGCTGGATTTACAGATGATTTTGAATGGAAT |
| | | | extracellular | TAATAATTACAAGAATCCCAAACTCACCAGGATGCTCACATTTAAGTT |
| | | | domain of IL-2 | TTACATGCCCAAGAAGGCCACAGAACTGAAACATCTTCAGTGTCTAG |
| | | | Rα | AAGAAGAACTCAAACCTCTGGAGGAAGTGCTAAATTTAGCTCAAAGC |
| | | | | AAAAACTTTCACTTAAGACCCAGGGACTTAATCAGCAATATCAACGTA |
| | | | | ATAGTTCTGGAACTAAAGGGATCTGAAACAACATTCATGTGTGAATA |
| | | | | TGCTGATGAGACAGCAACCATTGTAGAATTTCTGAACAGATGGATTA |
| | | | | CCTTTTGTCAAAGCATCATCTCAACACTGACTggtggaggtggatcaggtgga |
| | | | | ggtggatctggtggaggtggatcaggtggaggtggatccggtggaggtggatctGAGCT |
| | | | | CTGTGACGATGACCCGCCAGAGATCCCACACGCCACATTCAAAGCCA |
| | | | | TGGCCTACAAGGAAGGAACCATGTTGAACTGTGAATGCAAGAGAGG |
| | | | | TTTCCGCAGAATAAAAAGCGGGTCACTCTATATGCTCTGTACAGGAA |
| | | | | ACTCTAGCCACTCGTCCTGGGACAACCAATGTCAATGCACAAGCTCTG |
| | | | | CCACTCGGAACACAACGAAACAAGTGACACCTCAACCTGAAGAACAG |
| | | | | AAAGAAAGGAAAACCACAGAAATGCAAAGTCCAATGCAGCCAGTGG |
| | | | | ACCAAGCGAGCCTTCCAGGTCACTGCAGGGAACCTCCACCATGGGAA |
| | | | | AATGAAGCCACAGAGAGAATTTATCATTTCGTGGTGGGGCAGATGGT |
| | | | | TTATTATCAGTGCGTCCAGGGATACAGGGCTCTACACAGAGGTCCTG |
| | | | | CTGAGAGCGTCTGCAAAATGACCCACGGGAAGACAAGGTGGACCCA |
| | | | | GCCCCAGCTCATATGCACAGGTGAAATGGAGACCAGTCAGTTTCCAG |
| | | | | GTGAAGAGAAGCCTCAGGCAAGCCCCGAAGGCCGTCCTGAGAGTGA |
| | | | | GACTTCCTGCCTCGTCACAACAACAGATTTTCAAATACAGACAGAAAT |
| | | | | GGCTGCAACCATGGAGACGTCCATATTTACAACAGAGTACCAGGGTG |
| | | | | GACATCACCATCACCATCACTAATAA |
| 50 | AA | | (Gly4Ser)3 | GGGGSGGGGSGGGGS |
| | | | linker | |
| 51 | AA | | (Gly4Ser)2 | GGGGSGGGGS |
| | | | linker | |
| 52 | AA | | (Gly4Ser)1 | GGGGS |
| | | | linker | |
| 53 | NT | | Kozak | gccaccATGG |
| | | | sequence | |
| 54 | AA | Mouse | Unprocessed | MDSMQLASCVTLTLVLLVNSAPTSSSTSSSTAEAQQQQQQQQQQQQH |
| | | | form of IL-2 | LEQLLMDLQELLSRMENYRNLKLPRMLTFKFYLPKQATELKDLQCLEDEL |
| | | | (Gly4Ser)4- | GPLRHVLDLTQSKSFQLEDAENFISNIRVTVVKLKGSDNTFECQFDDESA |
| | | | extracellular | TVVDFLRRWIAFCQSIISTSPQGGGGSGGGGSGGGGSGGGGSELCLYDP |
| | | | domain of IL-2 | PEVPNATFKALSYKNGTILNCECKRGFRRLKELVYMRCLGNSWSSNCQC |
| | | | Rα + glycine | TSNSHDKSRKQVTAQLEHQKEQQTTTDMQKPTQSMHQENLTGHCRE |
| | | | spacer and | PPPWKHEDSKRIYHFVEGQSVHYECIPGYKALQRGPAISICKMKCGKTG |
| | | | poly-histidine | WTQPQLTCVDEREHHRFLASEESQGSRNSSPESETSCPITTTDFPQPTET |
| | | | region | TAMTETFVLTMEYKGGHHHHHH |
| 55 | AA | Mouse | Unprocessed | MDSMQLASCVTLTLVLLVNSAPTSSSTSSSTAEAQQQQQQQQQQQQH |
| | | | form of IL-2 | LEQLLMDLQELLSRMENYRNLKLPRMLTFKFYLPKQATELKDLQCLEDEL |
| | | | (Gly4Ser)5- | GPLRHVLDLTQSKSFQLEDAENFISNIRVTVVKLKGSDNTFECQFDDESA |
| | | | extracellular | TVVDFLRRWIAFCQSIISTSPQGGGGSGGGGSGGGGSGGGGSGGGGSE |
| | | | domain of IL-2 | LCLYDPPEVPNATFKALSYKNGTILNCECKRGFRRLKELVYMRCLGNSWS |
| | | | Rα + glycine | SNCQCTSNSHDKSRKQVTAQLEHQKEQQTTTDMQKPTQSMHQENLT |
| | | | spacer and | GHCREPPPWKHEDSKRIYHFVEGQSVHYECIPGYKALQRGPAISICKMKC |
| | | | poly-histidine | GKTGWTQPQLTCVDEREHHRFLASEESQGSRNSSPESETSCPITTTDFPQ |
| | | | region | PTETTAMTETFVLTMEYKGGHHHHHH |
| 56 | AA | Mouse | Unprocessed | MDSMQLASCVTLTLVLLVNSAPTSSSTSSSTAEAQQQQQQQQQQQQH |
| | | | form of IL-2 | LEQLLMDLQELLSRMENYRNLKLPRMLTFKFYLPKQATELKDLQCLEDEL |
| | | | (Gly3Ser)4- | GPLRHVLDLTQSKSFQLEDAENFISNIRVTVVKLKGSDNTFECQFDDESA |
| | | | extracellular | TVVDFLRRWIAFCQSIISTSPQGGGSGGGSGGGSGGGSELCLYDPPEVP |
| | | | domain of IL-2 | NATFKALSYKNGTILNCECKRGFRRLKELVYMRCLGNSWSSNCQCTSNS |
| | | | Rα + glycine | HDKSRKQVTAQLEHQKEQQTTTDMQKPTQSMHQENLTGHCREPPPW |
| | | | spacer and | KHEDSKRIYHFVEGQSVHYECIPGYKALQRGPAISICKMKCGKTGWTQP |
| | | | poly-histidine | QLTCVDEREHHRFLASEESQGSRNSSPESETSCPITTTDFPQPTETTAMTE |
| | | | region | TFVLTMEYKGGHHHHHH |
| 57 | AA | Mouse | Mature form of | MDSMQLASCVTLTLVLLVNSAPTSSSTSSSTAEAQQQQQQQQQQQQH |
| | | | IL-2 | LEQLLMDLQELLSRMENYRNLKLPRMLTFKFYLPKQATELKDLQCLEDEL |
| | | | (Gly3Ser)3- | GPLRHVLDLTQSKSFQLEDAENFISNIRVTVVKLKGSDNTFECQFDDESA |
| | | | extracellular | TVVDFLRRWIAFCQSIISTSPQGGGSGGGSGGGSELCLYDPPEVPNATFK |
| | | | domain of IL-2 | ALSYKNGTILNCECKRGFRRLKELVYMRCLGNSWSSNCQCTSNSHDKSR |
| | | | Rα + glycine | KQVTAQLEHQKEQQTTTDMQKPTQSMHQENLTGHCREPPPWKHEDS |
| | | | spacer and | KRIYHFVEGQSVHYECIPGYKALQRGPAISICKMKCGKTGWTQPQLTCV |
| | | | poly-histidine | DEREHHRFLASEESQGSRNSSPESETSCPITTTDFPQPTETTAMTETFVLT |
| | | | region | MEYKGGHHHHHH |
| 58 | AA | Human | Unprocessed | MDRMQLLSCIALSLALVTNSAPTSSSTKKTQLQLEHLLLDLQMILNGINN |
| | | | form IL-2 | YKNPKLTRMLTFKFYMPKKATELKHLQCLEEELKPLEEVLNLAQSKNFHL |
| | | | (Gly3Ser)2- | RPRDLISNINVIVLELKGSETTFMCEYADETATIVEFLNRWITFCQSIISTLT |
| | | | extracellular | GGGSGGGSELCDDDPPEIPHATFKAMAYKEGTMLNCECKRGFRRIKSGS |
| | | | domain of IL-2 | LYMLCTGNSSHSSWDNQCQCTSSATRNTTKQVTPQPEEQKERKTTEM |
| | | | Rα + glycine | QSPMQPVDQASLPGHCREPPPWENEATERIYHFVVGQMVYYQCVQG |
| | | | spacer and | YRALHRGPAESVCKMTHGKTRWTQPQLICTGEMETSQFPGEEKPQASP |
| | | | poly-histidine | EGRPESETSCLVTTTDFQIQTEMAATMETSIFTTEYQGGHHHHHH |
| | | | region | |
| 59 | AA | Human | Unprocessed | MDRMQLLSCIALSLALVTNSAPTSSSTKKTQLQLEHLLLDLQMILNGINN |
| | | | form of IL-2 | YKNPKLTRMLTFKFYMPKKATELKHLQCLEEELKPLEEVLNLAQSKNFHL |
| | | | (Gly3Ser)3- | RPRDLISNINVIVLELKGSETTFMCEYADETATIVEFLNRWITFCQSIISTLT |
| | | | extracellular | GGGSGGGSGGGSELCDDDPPEIPHATFKAMAYKEGTMLNCECKRGFRR |
| | | | domain of IL-2 | IKSGSLYMLCTGNSSHSSWDNQCQCTSSATRNTTKQVTPQPEEQKERKT |
| | | | Rα + glycine | TEMQSPMQPVDQASLPGHCREPPPWENEATERIYHFVVGQMVYYQC |
| | | | spacer and | VQGYRALHRGPAESVCKMTHGKTRWTQPQLICTGEMETSQFPGEEKP |
| | | | poly-histidine | QASPEGRPESETSCLVTTTDFQIQTEMAATMETSIFTTEYQGGHHHHHH |
| | | | region | |
| 60 | AA | Human | Unprocessed | MDRMQLLSCIALSLALVTNSAPTSSSTKKTQLQLEHLLLDLQMILNGINN |
| | | | form IL-2 | YKNPKLTRMLTFKFYMPKKATELKHLQCLEEELKPLEEVLNLAQSKNFHL |
| | | | (Gly3Ser)4- | RPRDLISNINVIVLELKGSETTFMCEYADETATIVEFLNRWITFCQSIISTLT |
| | | | extracellular | GGGSGGGSGGGSGGGSELCDDDPPEIPHATFKAMAYKEGTMLNCECK |
| | | | domain of IL-2 | RGFRRIKSGSLYMLCTGNSSHSSWDNQCQCTSSATRNTTKQVTPQPEE |
| | | | Rα + glycine | QKERKTTEMQSPMQPVDQASLPGHCREPPPWENEATERIYHFVVGQ |
| | | | spacer and | MVYYQCVQGYRALHRGPAESVCKMTHGKTRWTQPQLICTGEMETSQF |
| | | | poly-histidine | PGEEKPQASPEGRPESETSCLVTTTDFQIQTEMAATMETSIFTTEYQGGH |
| | | | region | HHHHH |
| 61 | AA | Human | Unprocessed | MDRMQLLSCIALSLALVTNSAPTSSSTKKTQLQLEHLLLDLQMILNGINN |
| | | | form IL-2 | YKNPKLTRMLTFKFYMPKKATELKHLQCLEEELKPLEEVLNLAQSKNFHL |
| | | | (Gly4Ser)4- | RPRDLISNINVIVLELKGSETTFMCEYADETATIVEFLNRWITFCQSIISTLT |
| | | | extracellular | GGGGSGGGGSGGGGSGGGGSELCDDDPPEIPHATFKAMAYKEGTML |
| | | | domain of IL-2 | NCECKRGFRRIKSGSLYMLCTGNSSHSSWDNQCQCTSSATRNTTKQVT |
| | | | Rα + glycine | PQPEEQKERKTTEMQSPMQPVDQASLPGHCREPPPWENEATERIYHFV |
| | | | spacer and | VGQMVYYQCVQGYRALHRGPAESVCKMTHGKTRWTQPQLICTGEME |
| | | | poly-histidine | TSQFPGEEKPQASPEGRPESETSCLVTTTDFQIQTEMAATMETSIFTTEY |
| | | | region | QGGHHHHHH |
| 62 | AA | Human | Mature form | APTSSSTKKTQLQLEHLLLDLQMILNGINNYKNPKLTRMLTFKFYMPKKA |
| | | | IL-2 | TELKHLQCLEEELKPLEEVLNLAQSKNFHLRPRDLISNINVIVLELKGSETTF |
| | | | (Gly3Ser)3- | MCEYADETATIVEFLNRWITFCQSIISTLTGGGSGGGSGGGSELCDDDPP |
| | | | extracellular | EIPHATFKAMAYKEGTMLNCECKRGFTSIKSGSLYMLCTGNSSHSSWDN |
| | | | domain of | QCQCTSSATRNTTKQVTPQPEEQKERKTTEMQSPMQPVDQASLPGHC |
| | | | mutIL-2 Rα | REPPPWENEATERIYHFVVGQMVYYQCVQGYRALHRGPAESVCKMTH |
| | | | | GKTRWTQPQLICTGEMETSQFPGEEKPQASPEGRPESETSCLVTTTDFQI |
| | | | | QTEMAATMETSIFTTEYQ |
| 63 | NT | Human | Unprocessed | ATGGACAGGATGCAACTCCTGTCTTGCATTGCACTAAGTCTTGCACTT |
| | | | form IL-2 | GTCACAAACAGTGCACCTACTTCAAGTTCTACAAAGAAAACACAGCT |
| | | | (Gly3Ser)3- | ACAACTGGAGCATTTACTGCTGGATTTACAGATGATTTTGAATGGAAT |
| | | | extracellular | TAATAATTACAAGAATCCCAAACTCACCAGGATGCTCACATTTAAGTT |
| | | | domain of | TTACATGCCCAAGAAGGCCACAGAACTGAAACATCTTCAGTGTCTAG |
| | | | mutIL-2 Rα | AAGAAGAACTCAAACCTCTGGAGGAAGTGCTAAATTTAGCTCAAAGC |
| | | | Mut | AAAAACTTTCACTTAAGACCCAGGGACTTAATCAGCAATATCAACGTA |
| | | | | ATAGTTCTGGAACTAAAGGGATCTGAAACAACATTCATGTGTGAATA |
| | | | | TGCTGATGAGACAGCAACCATTGTAGAATTTCTGAACAGATGGATTA |
| | | | | CCTTTGTCAAAGCATCATCTCAACACTGACTggtggaggttctggtggaggt |
| | | | | tcaggtggaggttcgGAGCTCTGTGACGATGACCCGCCAGAGATCCCACA |
| | | | | CGCCACATTCAAAGCCATGGCCTACAAGGAAGGAACCATGTTGAACT |
| | | | | GTGAATGCAAGAGAGGTTTCACCTCAATAAAAAGCGGGTCACTCTAT |
| | | | | ATGCTCTGTACAGGAAACTCTAGCCACTCGTCCTGGGACAACCAATG |
| | | | | TCAATGCACAAGCTCTGCCACTCGGAACACAACGAAACAAGTGACAC |
| | | | | CTCAACCTGAAGAACAGAAAGAAAGGAAAACCACAGAAATGCAAAG |
| | | | | TCCAATGCAGCCAGTGGACCAAGCGAGCCTTCCAGGTCACTGCAGGG |
| | | | | AACCTCCACCATGGGAAAATGAAGCCACAGAGAGAATTTATCATTTC |
| | | | | GTGGTGGGGCAGATGGTTTATTATCAGTGCGTCCAGGGATACAGGG |
| | | | | CTCTACACAGAGGTCCTGCTGAGAGCGTCTGCAAAATGACCCACGGG |
| | | | | AAGACAAGGTGGACCCAGCCCCAGCTCATATGCACAGGTGAAATGG |
| | | | | AGACCAGTCAGTTTCCAGGTGAAGAGAAGCCTCAGGCAAGCCCCGA |
| | | | | AGGCCGTCCTGAGAGTGAGACTTCCTGCCTCGTCACAACAACAGATT |
| | | | | TTCAAATACAGACAGAAATGGCTGCAACCATGGAGACGTCCATATTT |
| | | | | ACAACAGAGTACCAGGGTGGACATCACCATCACCATCACTAATAA |
| 64 | AA | Human | Mature form | APTSSSTKKTQLQLEHLLLDLQMILNGINNYKNPKLTRMLTFKFYMPKKA |
| | | | IL-2 | TELKHLQCLEEELKPLEEVLNLAQSKNFHLRPRDLISNINVIVLELKGSETTF |
| | | | (Gly4Ser)4- | MCEYADETATIVEFLNRWITFCQSIISTLTGGGGSGGGGSGGGGSGGGG |
| | | | extracellular | SELCDDDPPEIPHATFKAMAYKEGTMLNCECKRGFTSIKSGSLYMLCTG |
| | | | domain of | NSSHSSWDNQCQCTSSATRNTTKQVTPQPEEQKERKTTEMQSPMQPV |
| | | | mutIL-2 Rα | DQASLPGHCREPPPWENEATERIYHFVVGQMVYYQCVQGYRALHRGP |
| | | | | AESVCKMTHGKTRWTQPQLICTGEMETSQFPGEEKPQASPEGRPESET |
| | | | | SCLVTTTDFQIQTEMAATMETSIFTTEYQ |
| 65 | NT | Human | Unprocessed | ATGGACAGGATGCAACTCCTGTCTTGCATTGCACTAAGTCTTGCACTT |
| | | | form IL-2 | GTCACAAACAGTGCACCTACTTCAAGTTCTACAAAGAAAACACAGCT |
| | | | (Gly4Ser)4- | ACAACTGGAGCATTTACTGCTGGATTTACAGATGATTTTGAATGGAAT |
| | | | extracellular | TAATAATTACAAGAATCCCAAACTCACCAGGATGCTCACATTTAAGTT |
| | | | domain of | TTACATGCCCAAGAAGGCCACAGAACTGAAACATCTTCAGTGTCTAG |
| | | | mutIL-2 Rα | AAGAAGAACTCAAACCTCTGGAGGAAGTGCTAAATTTAGCTCAAAGC |
| | | | | AAAAACTTTCACTTAAGACCCAGGGACTTAATCAGCAATATCAACGTA |
| | | | | ATAGTTCTGGAACTAAAGGGATCTGAAACAACATTCATGTGTGAATA |
| | | | | TGCTGATGAGACAGCAACCATTGTAGAATTTCTGAACAGATGGATTA |
| | | | | CCTTTTGTCAAAGCATCATCTCAACACTGACTggtggaggtggatctggtgga |
| | | | | ggtggatcaggtggaggtggatccggtggaggtggatct |
| | | | | GAGCTCTGTGACGATGACCCGCCAGAGATCCCACACGCCACATTCAA |
| | | | | AGCCATGGCCTACAAGGAAGGAACCATGTTGAACTGTGAATGCAAG |
| | | | | AGAGGTTTCACCTCAATAAAAAGCGGGTCACTCTATATGCTCTGTACA |
| | | | | GGAAACTCTAGCCACTCGTCCTGGGACAACCAATGTCAATGCACAAG |
| | | | | CTCTGCCACTCGGAACACAACGAAACAAGTGACACCTCAACCTGAAG |
| | | | | AACAGAAAGAAAGGAAAACCACAGAAATGCAAAGTCCAATGCAGCC |
| | | | | AGTGGACCAAGCGAGCCTTCCAGGTCACTGCAGGGAACCTCCACCAT |
| | | | | GGGAAAATGAAGCCACAGAGAGAATTTATCATTTCGTGGTGGGGCA |
| | | | | GATGGTTTATTATCAGTGCGTCCAGGGATACAGGGCTCTACACAGAG |
| | | | | GTCCTGCTGAGAGCGTCTGCAAAATGACCCACGGGAAGACAAGGTG |
| | | | | GACCCAGCCCCAGCTCATATGCACAGGTGAAATGGAGACCAGTCAGT |
| | | | | TTCCAGGTGAAGAGAAGCCTCAGGCAAGCCCCGAAGGCCGTCCTGA |
| | | | | GAGTGAGACTTCCTGCCTCGTCACAACAACAGATTTTCAAATACAGAC |
| | | | | AGAAATGGCTGCAACCATGGAGACGTCCATATTTACAACAGAGTACC |
| | | | | AGGGTGGACATCACCATCACCATCACTAATAA |

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

That which is claimed:

1. A fusion protein comprising:
(a) a first polypeptide comprising Interleukin-2 (IL-2) or a functional variant or fragment thereof; and
(b) a second polypeptide, fused in frame to said first polypeptide, said second polypeptide comprises an extracellular domain of Interleukin-2 Receptor alpha (IL-2Rα) or a functional variant or fragment thereof,
wherein said fusion protein has IL-2 activity.

2. The fusion protein of claim 1, wherein said fusion protein has an increased IL-2 potency when compared to native or recombinant IL-2.

3. The fusion protein of any one of claim 1 or 2, wherein said fusion protein has an increased persistent IL-2 stimulation of IL-2R bearing lymphocytes in vivo when compared to native or recombinant IL-2.

4. The fusion protein of any of one of claims 1-3, wherein
(a) said first polypeptide comprising IL-2 shares at least 70% sequence identity to SEQ ID NO: 2; and/or,
(b) said second polypeptide comprising the extracellular domain of IL-2Rα shares at least 70% sequence identity to SEQ ID NO: 7.

5. The fusion protein of claim 4, wherein
(a) said first polypeptide comprising IL-2 shares at least 85%, 90%, 95%, or 99% sequence identity to SEQ ID NO: 2; and/or,
(b) said second polypeptide comprising the extracellular domain of IL-2Rα shares at least 85%, 90%, 95% or 99% sequence identity to SEQ ID NO: 7.

6. The fusion protein of any one of claims 1-5, further comprising a linker sequence fused in frame between said first polypeptide and said second polypeptide.

7. The fusion protein of claim 6, wherein the linker sequence comprises:
(a) a glycine/serine linker;
(b) the sequence set forth in SEQ ID NO:11, 12, 13, 14, 15, 40, 41, 50, 51, or 52; or,
(c) a sequence having least 90% or 95% sequence identity to any one of SEQ ID NO: 11, 12, 13, 14, 15, 40, 41, 50, 51, or 52.

8. The fusion protein of claim 7, wherein the fusion protein comprises
(a) the amino acid sequence of any one of SEQ ID NO: 16, 17, 18, 19, 20, 21, 22, 23, 24, 25, 26, 27, 36, 37, 38, 39, 43, 44, 45, 46, 54, 55, 56, 57, 58, 59, 60, or 61; or,
(b) a sequence having at least 80%, 85%, 90%, or 95% to any one of SEQ ID NO: 16, 17, 18, 19, 20, 21, 22, 23, 24, 25, 26, 27, 36, 37, 38, 39, 43, 44, 45, 46, 54, 55, 56, 57, 58, 59, 60, or 61.

9. The fusion protein of claim 7, wherein the fusion protein comprises at least one mutation in the extracellular domain of IL-2Rα.

10. The fusion protein of claim 9, wherein the fusion protein comprises:
(a) the amino acid sequence of any one of SEQ ID NO: 62 or 64; or
(b) a sequence having at least 80%, 85%, 90%, or 95% to any one of SEQ ID NO: 62 or 64.

11. A polynucleotide comprising a nucleotide sequence encoding the fusion protein of any one of claims 1-10.

12. A host cell comprising the polynucleotide of claim 11.

13. The host cell of claim 12, wherein the host cell comprises a CHO cell or a COS cell.

14. A method for making a fusion protein of any one of claims 1-10, said method comprising introducing into a host cell a polynucleotide encoding the fusion protein of any one of claims 1-8 and expressing the fusion protein in the host cell.

15. The method of claim 14, wherein the polynucleotide encoding the fusion protein is operably linked to a promoter active in the host cell.

16. A method for decreasing the immune response in a subject comprising administering to a subject in need of a decrease in the immune response a therapeutically effective amount of the fusion protein of any one of claims 1-10.

17. The method of claim 16, wherein said subject has an autoimmune disease.

18. The method of claim 17, wherein said autoimmune disease comprises type 1 diabetes, multiple sclerosis, rheumatoid arthritis, celiac disease, systemic lupus erythematous, juvenile idiopathic arthritis, Crohn's disease, ulcerative colitis or systemic sclerosis, graft versus host disease, psoriasis, alopecia areata, or HCV-induced vasculitis.

19. The method of any one of claims 16-18, wherein the therapeutically effective amount of the fusion protein comprises 10³ to 10⁶ IU of IL-2 activity per adult or 10⁴±100 fold of IL-2 activity per adult to decrease an immune response.

20. A method for increasing the immune response in a subject comprising administering to a subject in need of an increase in the immune response a therapeutically effective amount of the fusion protein of any one of claims 1-10.

21. A method of enhancing the immunogenicity of a vaccine in a subject, comprising:
(a) administering to the subject a therapeutically effective amount of the fusion protein of any one of claims 1-10; and,
(b) administering to the subject a vaccine,
wherein said fusion protein enhances the immunogenicity of the vaccine.

22. A method of overcoming a suppressed immune response to a vaccine in a subject, comprising:
(a) administering to the subject a therapeutically effective amount of the fusion protein of any one of claims 1-10; and,
(b) administering to the subject a vaccine,
wherein said fusion protein overcomes said suppressed immune response to said vaccine.

23. The method of claim 21 or 22, wherein administration of said therapeutically effective amount of the fusion protein and administration of said vaccine is sequential, in any order.

24. The method of claim 21 or 22, wherein administration of said therapeutically effective amount of said fusion protein and administration of the vaccine is simultaneous.

25. The method of any one of claims 21-24, wherein said vaccine is a cancer vaccine.

26. The method of any one of claims 20-25, wherein the therapeutically effective amount of the fusion protein comprises at least 10⁴ to 10⁷ IU of IL-2 activity per adult or at least 10⁵±100 of IL-2 activity per adult to increase an immune response.

27. The method of any one of claims 14-26, wherein said subject is a human.

28. The method of any one of claims 14-26, wherein said subject is a domesticated mammal or an agricultural mammal.


SEQUENCE LISTING (65 sequences)

SEQ ID NO: 1 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 2 - protein, 133 aa, Homo sapiens [sequence omitted]

SEQ ID NO: 3 - protein, 169 aa, Mus musculus
MYSMQLASCVTLTLVLLVNSAPTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQ

SEQ ID NO: 4 - protein, 149 aa, Mus musculus
APTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQ

SEQ ID NO: 5 - protein, 272 aa, Homo sapiens
MDSYLLMWGLLTFIMVPGCQAELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 6 - protein, 251 aa, Homo sapiens
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 7 - protein, 219 aa, Homo sapiens
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQ

SEQ ID NO: 8 - protein, 268 aa, Mus musculus
MEPRLLMLGFLSLTIVPSCRAELCLYDPPEVPNATFKALSYKNGTILNCECKRGFRRLKELVYMRCLGNSWSSNCQCTSNSHDKSRKQVTAQLEHQKEQQTTTDMQKPTQSMHQENLTGHCREPPPWKHEDSKRIYHFVEGQSVHYECIPGYKALQRGPAISICKMKCGKTGWTQPQLTCVDEREHHRFLASEESQGSRNSSPESETSCPITTTDFPQPTETTAMTETFVLTMEYKVAVASCLFLLISILLLSGLTWQHRWRKSRRTI

SEQ ID NO: 9 - protein, 247 aa, Mus musculus
ELCLYDPPEVPNATFKALSYKNGTILNCECKRGFRRLKELVYMRCLGNSWSSNCQCTSNSHDKSRKQVTAQLEHQKEQQTTTDMQKPTQSMHQENLTGHCREPPPWKHEDSKRIYHFVEGQSVHYECIPGYKALQRGPAISICKMKCGKTGWTQPQLTCVDEREHHRFLASEESQGSRNSSPESETSCPITTTDFPQPTETTAMTETFVLTMEYKVAVASCLFLLISILLLSGLTWQHRWRKSRRTI

SEQ ID NO: 10 - protein, 215 aa, Mus musculus
ELCLYDPPEVPNATFKALSYKNGTILNCECKRGFRRLKELVYMRCLGNSWSSNCQCTSNSHDKSRKQVTAQLEHQKEQQTTTDMQKPTQSMHQENLTGHCREPPPWKHEDSKRIYHFVEGQSVHYECIPGYKALQRGPAISICKMKCGKTGWTQPQLTCVDEREHHRFLASEESQGSRNSSPESETSCPITTTDFPQPTETTAMTETFVLTMEYK

SEQ ID NO: 11 - protein, 16 aa, synthetic construct
  REGION 1..16: note = (Gly3Ser)4 linker
GGGSGGGSGGGSGGGS

SEQ ID NO: 12 - protein, 8 aa, synthetic construct
  REGION 1..8: note = (Gly3Ser)2 linker
GGGSGGGS

SEQ ID NO: 13 - protein, 12 aa, synthetic construct
  REGION 1..12: note = (Gly3Ser)3 linker
GGGSGGGSGGGS

SEQ ID NO: 14 - protein, 20 aa, synthetic construct
  REGION 1..20: note = (Gly3Ser)5 linker
GGGSGGGSGGGSGGGSGGGS

SEQ ID NO: 15 - skipped

SEQ ID NO: 16 - protein, 384 aa, synthetic construct
  REGION 1..384: note = Synthesized Mature form of IL-2 (Gly4Ser)4-extracellular domainof IL-2 R
APTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQGGGGSGGGGSGGGGSGGGGSELCLYDPPEVPNATFKALSYKNGTILNCECKRGFRRLKELVYMRCLGNSWSSNCQCTSNSHDKSRKQVTAQLEHQKEQQTTTDMQKPTQSMHQENLTGHCREPPPWKHEDSKRIYHFVEGQSVHYECIPGYKALQRGPAISICKMKCGKTGWTQPQLTCVDEREHHRFLASEESQGSRNSSPESETSCPITTTDFPQPTETTAMTETFVLTMEYK

SEQ ID NO: 17 - protein, 404 aa, synthetic construct
  REGION 1..404: note = Synthesized unprocessed form of IL-2 (Gly4Ser)4-extracellulardomain of IL-2 R
MDSMQLASCVTLTLVLLVNSAPTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQGGGGSGGGGSGGGGSGGGGSELCLYDPPEVPNATFKALSYKNGTILNCECKRGFRRLKELVYMRCLGNSWSSNCQCTSNSHDKSRKQVTAQLEHQKEQQTTTDMQKPTQSMHQENLTGHCREPPPWKHEDSKRIYHFVEGQSVHYECIPGYKALQRGPAISICKMKCGKTGWTQPQLTCVDEREHHRFLASEESQGSRNSSPESETSCPITTTDFPQPTETTAMTETFVLTMEYK

SEQ ID NO: 18 - protein, 389 aa, synthetic construct
  REGION 1..389: note = Synthesized mature form of IL-2 (Gly4Ser)5- extracellular domainof IL-2 R
APTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQGGGGSGGGGSGGGGSGGGGSGGGGSELCLYDPPEVPNATFKALSYKNGTILNCECKRGFRRLKELVYMRCLGNSWSSNCQCTSNSHDKSRKQVTAQLEHQKEQQTTTDMQKPTQSMHQENLTGHCREPPPWKHEDSKRIYHFVEGQSVHYECIPGYKALQRGPAISICKMKCGKTGWTQPQLTCVDEREHHRFLASEESQGSRNSSPESETSCPITTTDFPQPTETTAMTETFVLTMEYK

SEQ ID NO: 19 - protein, 409 aa, synthetic construct
  REGION 1..409: note = Synthesized unprocessed form of IL-2 (Gly4Ser)5- extracellulardomain of IL-2 R
MDSMQLASCVTLTLVLLVNSAPTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQGGGGSGGGGSGGGGSGGGGSGGGGSELCLYDPPEVPNATFKALSYKNGTILNCECKRGFRRLKELVYMRCLGNSWSSNCQCTSNSHDKSRKQVTAQLEHQKEQQTTTDMQKPTQSMHQENLTGHCREPPPWKHEDSKRIYHFVEGQSVHYECIPGYKALQRGPAISICKMKCGKTGWTQPQLTCVDEREHHRFLASEESQGSRNSSPESETSCPITTTDFPQPTETTAMTETFVLTMEYK

SEQ ID NO: 20 - protein, 380 aa, synthetic construct
  REGION 1..380: note = Synthesized mature form of IL-2 (Gly3Ser)4- extracellular domainof IL-2 R
APTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQGGGSGGGSGGGSGGGSELCLYDPPEVPNATFKALSYKNGTILNCECKRGFRRLKELVYMRCLGNSWSSNCQCTSNSHDKSRKQVTAQLEHQKEQQTTTDMQKPTQSMHQENLTGHCREPPPWKHEDSKRIYHFVEGQSVHYECIPGYKALQRGPAISICKMKCGKTGWTQPQLTCVDEREHHRFLASEESQGSRNSSPESETSCPITTTDFPQPTETTAMTETFVLTMEYK

SEQ ID NO: 21 - protein, 400 aa, synthetic construct
  REGION 1..400: note = Synthesized unprocessed form of IL-2 (Gly3Ser)4- extracellulardomain of IL-2 R
MDSMQLASCVTLTLVLLVNSAPTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQGGGSGGGSGGGSGGGSELCLYDPPEVPNATFKALSYKNGTILNCECKRGFRRLKELVYMRCLGNSWSSNCQCTSNSHDKSRKQVTAQLEHQKEQQTTTDMQKPTQSMHQENLTGHCREPPPWKHEDSKRIYHFVEGQSVHYECIPGYKALQRGPAISICKMKCGKTGWTQPQLTCVDEREHHRFLASEESQGSRNSSPESETSCPITTTDFPQPTETTAMTETFVLTMEYK

SEQ ID NO: 22 - protein, 372 aa, synthetic construct
  REGION 1..372: note = Synthesized mature form IL-2 (Gly4Ser)4- extracellular domain ofIL-2 R
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQ

SEQ ID NO: 23 - protein, 392 aa, synthetic construct
  REGION 1..392: note = Synthesized unprocessed form IL-2 (Gly4Ser)4- extracellulardomain of IL-2 R
MDRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQ

SEQ ID NO: 24 - protein, 368 aa, synthetic construct
  REGION 1..368: note = Synthesized mature form IL-2 (Gly3Ser)4- extracellular domain ofIL-2 R
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGSGGGSGGGSGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQ

SEQ ID NO: 25 - protein, 388 aa, synthetic construct
  REGION 1..388: note = Synthesized unprocessed form IL-2 (Gly3Ser)4- extracellulardomain of IL-2 R
MDRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGSGGGSGGGSGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQ

SEQ ID NO: 26 - protein, 364 aa, synthetic construct
  REGION 1..364: note = Synthesized mature form IL-2 (Gly3Ser)3- extracellular domain ofIL-2 R
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGSGGGSGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQ

SEQ ID NO: 27 - protein, 384 aa, synthetic construct
  REGION 1..384: note = Synthesized unprocessed form of IL-2 (Gly3Ser)3- extracellulardomain of IL-2 R
MDRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGSGGGSGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQ

SEQ ID NO: 28 - DNA, 66 bp, synthetic construct
  misc_feature 1..66: note = IL-2 leader optimized Kozak sequence
  source 1..66: mol_type = other DNA
gccaccatggacaggatgcaactcctgtcttgcattgcactaagtcttgcacttgtcacaaacagt

SEQ ID NO: 29 - DNA, 1239 bp, synthetic construct
  misc_feature 1..1239: note = Synthesized unprocessed form of IL-2 (Gly4Ser)4-extracellulardomain of IL-2 R
  source 1..1239: mol_type = other DNA
atggacagcatgcagctcgcatcctgtgtcacattgacacttgtgctccttgtcaacagcgcacccacttcaagctctacttcaagctctacagcggaagcacagcagcagcagcagcagcagcagcagcagcagcacctggagcagctgttgatggacctacaggagctcctgagcaggatggagaattacaggaacctgaaactccccaggatgctcaccttcaaattttacttgcccaagcaggccacagaattgaaagatcttcagtgcctagaagatgaacttggacctctgcggcatgttctggatttgactcaaagcaaaagctttcaattggaagatgctgagaatttcatcagcaatatcagagtaactgttgtaaaactaaagggctctgacaacacatttgagtgccaattcgatgatgagtcagcaactgtggtggactttctgaggagatggatagccttctgtcaaagcatcatctcaacaagccctcaaggtggaggtggatctggtggaggtggatcaggtggaggtggatccggtggaggtggatctgaactgtgtctgtatgacccacccgaggtccccaatgccacattcaaagccctctcctacaagaacggcaccatcctaaactgtgaatgcaagagaggtttccgaagactaaaggaattggtctatatgcgttgcttaggaaactcctggagcagcaactgccagtgcaccagcaactcccatgacaaatcgagaaagcaagttacagctcaacttgaacaccagaaagagcaacaaaccacaacagacatgcagaagccaacacagtctatgcaccaagagaaccttacaggtcactgcagggagccacctccttggaaacatgaagattccaagagaatctatcatttcgtggaaggacagagtgttcactacgagtgtattccgggatacaaggctctacagagaggtcctgctattagcatctgcaagatgaagtgtgggaaaacggggtggactcagccccagctcacatgtgtagatgaaagagaacaccaccgatttctggctagtgaggaatctcaaggaagcagaaattcttctcccgagagtgagacttcctgccccataaccaccacagacttcccacaacccacagaaacaactgcaatgacggagacatttgtgctcacaatggagtataagggtggacatcaccatcaccatcactaataa

SEQ ID NO: 30 - DNA, 1227 bp, synthetic construct
  misc_feature 1..1227: note = Synthesized unprocessed form of IL-2 (Gly3Ser)4- extracellulardomain of IL-2 R
  source 1..1227: mol_type = other DNA
atggacagcatgcagctcgcatcctgtgtcacattgacacttgtgctccttgtcaacagcgcacccacttcaagctctacttcaagctctacagcggaagcacagcagcagcagcagcagcagcagcagcagcagcacctggagcagctgttgatggacctacaggagctcctgagcaggatggagaattacaggaacctgaaactccccaggatgctcaccttcaaattttacttgcccaagcaggccacagaattgaaagatcttcagtgcctagaagatgaacttggacctctgcggcatgttctggatttgactcaaagcaaaagctttcaattggaagatgctgagaatttcatcagcaatatcagagtaactgttgtaaaactaaagggctctgacaacacatttgagtgccaattcgatgatgagtcagcaactgtggtggactttctgaggagatggatagccttctgtcaaagcatcatctcaacaagccctcaaggtggaggttctggtggaggttcaggtggaggttcgggtggaggttctgaactgtgtctgtatgacccacccgaggtccccaatgccacattcaaagccctctcctacaagaacggcaccatcctaaactgtgaatgcaagagaggtttccgaagactaaaggaattggtctatatgcgttgcttaggaaactcctggagcagcaactgccagtgcaccagcaactcccatgacaaatcgagaaagcaagttacagctcaacttgaacaccagaaagagcaacaaaccacaacagacatgcagaagccaacacagtctatgcaccaagagaaccttacaggtcactgcagggagccacctccttggaaacatgaagattccaagagaatctatcatttcgtggaaggacagagtgttcactacgagtgtattccgggatacaaggctctacagagaggtcctgctattagcatctgcaagatgaagtgtgggaaaacggggtggactcagccccagctcacatgtgtagatgaaagagaacaccaccgatttctggctagtgaggaatctcaaggaagcagaaattcttctcccgagagtgagacttcctgccccataaccaccacagacttcccacaacccacagaaacaactgcaatgacggagacatttgtgctcacaatggagtataagggtggacatcaccatcaccatcactaataa

SEQ ID NO: 31 - DNA, 1254 bp, synthetic construct
  misc_feature 1..1254: note = Synthesized unprocessed form of IL-2 (Gly4Ser)5- extracellulardomain of IL-2 R
  source 1..1254: mol_type = other DNA
atggacagcatgcagctcgcatcctgtgtcacattgacacttgtgctccttgtcaacagcgcacccacttcaagctctacttcaagctctacagcggaagcacagcagcagcagcagcagcagcagcagcagcagcacctggagcagctgttgatggacctacaggagctcctgagcaggatggagaattacaggaacctgaaactccccaggatgctcaccttcaaattttacttgcccaagcaggccacagaattgaaagatcttcagtgcctagaagatgaacttggacctctgcggcatgttctggatttgactcaaagcaaaagctttcaattggaagatgctgagaatttcatcagcaatatcagagtaactgttgtaaaactaaagggctctgacaacacatttgagtgccaattcgatgatgagtcagcaactgtggtggactttctgaggagatggatagccttctgtcaaagcatcatctcaacaagccctcaaggtggaggtggatcaggtggaggtggatctggtggaggtggatcaggtggaggtggatccggtggaggtggatctgaactgtgtctgtatgacccacccgaggtccccaatgccacattcaaagccctctcctacaagaacggcaccatcctaaactgtgaatgcaagagaggtttccgaagactaaaggaattggtctatatgcgttgcttaggaaactcctggagcagcaactgccagtgcaccagcaactcccatgacaaatcgagaaagcaagttacagctcaacttgaacaccagaaagagcaacaaaccacaacagacatgcagaagccaacacagtctatgcaccaagagaaccttacaggtcactgcagggagccacctccttggaaacatgaagattccaagagaatctatcatttcgtggaaggacagagtgttcactacgagtgtattccgggatacaaggctctacagagaggtcctgctattagcatctgcaagatgaagtgtgggaaaacggggtggactcagccccagctcacatgtgtagatgaaagagaacaccaccgatttctggctagtgaggaatctcaaggaagcagaaattcttctcccgagagtgagacttcctgccccataaccaccacagacttcccacaacccacagaaacaactgcaatgacggagacatttgtgctcacaatggagtataagggtggacatcaccatcaccatcactaataa

SEQ ID NO: 32 - DNA, 1206 bp, synthetic construct
  misc_feature 1..1206: note = Synthesized unprocessed form IL-2 (Gly4Ser)4- extracellulardomain of IL-2 R
  source 1..1206: mol_type = other DNA
atggacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgactggtggaggtggatctggtggaggtggatcaggtggaggtggatccggtggaggtggatctgagctctgtgacgatgacccgccagagatcccacacgccacattcaaagccatggcctacaaggaaggaaccatgttgaactgtgaatgcaagagaggtttccgcagaataaaaagcgggtcactctatatgctctgtacaggaaactctagccactcgtcctgggacaaccaatgtcaatgcacaagctctgccactcggaacacaacgaaacaagtgacacctcaacctgaagaacagaaagaaaggaaaaccacagaaatgcaaagtccaatgcagccagtggaccaagcgagccttccaggtcactgcagggaacctccaccatgggaaaatgaagccacagagagaatttatcatttcgtggtggggcagatggtttattatcagtgcgtccagggatacagggctctacacagaggtcctgctgagagcgtctgcaaaatgacccacgggaagacaaggtggacccagccccagctcatatgcacaggtgaaatggagaccagtcagtttccaggtgaagagaagcctcaggcaagccccgaaggccgtcctgagagtgagacttcctgcctcgtcacaacaacagattttcaaatacagacagaaatggctgcaaccatggagacgtccatatttacaacagagtaccagggtggacatcaccatcaccatcactaataa

SEQ ID NO: 33 - DNA, 1194 bp, synthetic construct
  misc_feature 1..1194: note = Synthesized unprocessed form IL-2 (Gly3Ser)4- extracellulardomain of IL-2 R
  source 1..1194: mol_type = other DNA
atggacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgactggtggaggttctggtggaggttcaggtggaggttcgggtggaggttctgagctctgtgacgatgacccgccagagatcccacacgccacattcaaagccatggcctacaaggaaggaaccatgttgaactgtgaatgcaagagaggtttccgcagaataaaaagcgggtcactctatatgctctgtacaggaaactctagccactcgtcctgggacaaccaatgtcaatgcacaagctctgccactcggaacacaacgaaacaagtgacacctcaacctgaagaacagaaagaaaggaaaaccacagaaatgcaaagtccaatgcagccagtggaccaagcgagccttccaggtcactgcagggaacctccaccatgggaaaatgaagccacagagagaatttatcatttcgtggtggggcagatggtttattatcagtgcgtccagggatacagggctctacacagaggtcctgctgagagcgtctgcaaaatgacccacgggaagacaaggtggacccagccccagctcatatgcacaggtgaaatggagaccagtcagtttccaggtgaagagaagcctcaggcaagccccgaaggccgtcctgagagtgagacttcctgcctcgtcacaacaacagattttcaaatacagacagaaatggctgcaaccatggagacgtccatatttacaacagagtaccagggtggacatcaccatcaccatcactaataa

SEQ ID NO: 34 - DNA, 1182 bp, synthetic construct
  misc_feature 1..1182: note = Synthesized unprocessed form of IL-2 (Gly3Ser)3- extracellulardomain of IL-2 R
  source 1..1182: mol_type = other DNA
atggacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgactggtggaggttctggtggaggttcaggtggaggttcggagctctgtgacgatgacccgccagagatcccacacgccacattcaaagccatggcctacaaggaaggaaccatgttgaactgtgaatgcaagagaggtttccgcagaataaaaagcgggtcactctatatgctctgtacaggaaactctagccactcgtcctgggacaaccaatgtcaatgcacaagctctgccactcggaacacaacgaaacaagtgacacctcaacctgaagaacagaaagaaaggaaaaccacagaaatgcaaagtccaatgcagccagtggaccaagcgagccttccaggtcactgcagggaacctccaccatgggaaaatgaagccacagagagaatttatcatttcgtggtggggcagatggtttattatcagtgcgtccagggatacagggctctacacagaggtcctgctgagagcgtctgcaaaatgacccacgggaagacaaggtggacccagccccagctcatatgcacaggtgaaatggagaccagtcagtttccaggtgaagagaagcctcaggcaagccccgaaggccgtcctgagagtgagacttcctgcctcgtcacaacaacagattttcaaatacagacagaaatggctgcaaccatggagacgtccatatttacaacagagtaccagggtggacatcaccatcaccatcactaataa

SEQ ID NO: 35 - RNA, 13 nt, synthetic construct
  source 1..13: mol_type = other RNA
  misc_feature 1..13: note = Kozak consensus
gccgccrccatgg

SEQ ID NO: 36 - protein, 396 aa, synthetic construct
  REGION 1..396: note = Synthesized unprocessed form of IL-2 (Gly3Ser)3- extracellulardomain of IL-2 R
MYSMQLASCVTLTLVLLVNSAPTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQGGGSGGGSGGGSELCLYDPPEVPNATFKALSYKNGTILNCECKRGFRRLKELVYMRCLGNSWSSNCQCTSNSHDKSRKQVTAQLEHQKEQQTTTDMQKPTQSMHQENLTGHCREPPPWKHEDSKRIYHFVEGQSVHYECIPGYKALQRGPAISICKMKCGKTGWTQPQLTCVDEREHHRFLASEESQGSRNSSPESETSCPITTTDFPQPTETTAMTETFVLTMEYK

SEQ ID NO: 37 - protein, 396 aa, synthetic construct
  REGION 1..396: note = Synthesized mature form of IL-2 (Gly3Ser)3- extracellular domainof IL-2 R
MDSMQLASCVTLTLVLLVNSAPTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQGGGSGGGSGGGSELCLYDPPEVPNATFKALSYKNGTILNCECKRGFRRLKELVYMRCLGNSWSSNCQCTSNSHDKSRKQVTAQLEHQKEQQTTTDMQKPTQSMHQENLTGHCREPPPWKHEDSKRIYHFVEGQSVHYECIPGYKALQRGPAISICKMKCGKTGWTQPQLTCVDEREHHRFLASEESQGSRNSSPESETSCPITTTDFPQPTETTAMTETFVLTMEYK

SEQ ID NO: 38 - protein, 397 aa, synthetic construct
  REGION 1..397: note = Synthesized unprocessed form of IL-2 (Gly4Ser)5- extracellulardomain of IL-2 R
MDRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSGGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQ

SEQ ID NO: 39 - protein, 377 aa, synthetic construct
  REGION 1..377: note = Synthesized mature form of human IL-2 (Gly4Ser)5- extracellulardomain of IL-2 R
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSGGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQ

SEQ ID NO: 40 - protein, 20 aa, synthetic construct
  REGION 1..20: note = Linker sequence (Gly4Ser)4
GGGGSGGGGSGGGGSGGGGS

SEQ ID NO: 41 - protein, 25 aa, synthetic construct
  REGION 1..25: note = Linker sequence (Gly4Ser)5
GGGGSGGGGSGGGGSGGGGSGGGGS

SEQ ID NO: 42 - DNA, 1215 bp, synthetic construct
  misc_feature 1..1215: note = Synthesized unprocessed form IL-2 (Gly3Ser)3- extracellulardomain of IL-2 R
  source 1..1215: mol_type = other DNA
atggacagcatgcagctcgcatcctgtgtcacattgacacttgtgctccttgtcaacagcgcacccacttcaagctctacttcaagctctacagcggaagcacagcagcagcagcagcagcagcagcagcagcagcacctggagcagctgttgatggacctacaggagctcctgagcaggatggagaattacaggaacctgaaactccccaggatgctcaccttcaaattttacttgcccaagcaggccacagaattgaaagatcttcagtgcctagaagatgaacttggacctctgcggcatgttctggatttgactcaaagcaaaagctttcaattggaagatgctgagaatttcatcagcaatatcagagtaactgttgtaaaactaaagggctctgacaacacatttgagtgccaattcgatgatgagtcagcaactgtggtggactttctgaggagatggatagccttctgtcaaagcatcatctcaacaagccctcaaggtggaggttctggtggaggttcaggtggaggttcggaactgtgtctgtatgacccacccgaggtccccaatgccacattcaaagccctctcctacaagaacggcaccatcctaaactgtgaatgcaagagaggtttccgaagactaaaggaattggtctatatgcgttgcttaggaaactcctggagcagcaactgccagtgcaccagcaactcccatgacaaatcgagaaagcaagttacagctcaacttgaacaccagaaagagcaacaaaccacaacagacatgcagaagccaacacagtctatgcaccaagagaaccttacaggtcactgcagggagccacctccttggaaacatgaagattccaagagaatctatcatttcgtggaaggacagagtgttcactacgagtgtattccgggatacaaggctctacagagaggtcctgctattagcatctgcaagatgaagtgtgggaaaacggggtggactcagccccagctcacatgtgtagatgaaagagaacaccaccgatttctggctagtgaggaatctcaaggaagcagaaattcttctcccgagagtgagacttcctgccccataaccaccacagacttcccacaacccacagaaacaactgcaatgacggagacatttgtgctcacaatggagtataagggtggacatcaccatcaccatcactaataa

SEQ ID NO: 43 - protein, 360 aa, synthetic construct
  REGION 1..360: note = Synthesized mature form IL-2 (Gly3Ser)2- extracellular domain ofIL-2 R
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGSGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQ

SEQ ID NO: 44 - protein, 380 aa, synthetic construct
  REGION 1..380: note = Synthesized unprocessed form IL-2 (Gly3Ser)2- extracellulardomain of IL-2 R
MDRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGSGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQ

SEQ ID NO: 45 - protein, 355 aa, synthetic construct
  REGION 1..355: note = Synthesized mature form IL-2 (Gly3)- extracellular domain of IL-2R
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQ

SEQ ID NO: 46 - protein, 375 aa, synthetic construct
  REGION 1..375: note = Synthesized unprocessed form IL-2 (Gly3)- extracellular domain ofIL-2 R
MDRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQ

SEQ ID NO: 47 - DNA, 1170 bp, synthetic construct
  misc_feature 1..1170: note = Synthesized unprocessed form IL-2 (Gly3Ser)2- extracellulardomain of IL-2 R
  source 1..1170: mol_type = other DNA
atggacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgactggtggaggttctggtggaggttcagagctctgtgacgatgacccgccagagatcccacacgccacattcaaagccatggcctacaaggaaggaaccatgttgaactgtgaatgcaagagaggtttccgcagaataaaaagcgggtcactctatatgctctgtacaggaaactctagccactcgtcctgggacaaccaatgtcaatgcacaagctctgccactcggaacacaacgaaacaagtgacacctcaacctgaagaacagaaagaaaggaaaaccacagaaatgcaaagtccaatgcagccagtggaccaagcgagccttccaggtcactgcagggaacctccaccatgggaaaatgaagccacagagagaatttatcatttcgtggtggggcagatggtttattatcagtgcgtccagggatacagggctctacacagaggtcctgctgagagcgtctgcaaaatgacccacgggaagacaaggtggacccagccccagctcatatgcacaggtgaaatggagaccagtcagtttccaggtgaagagaagcctcaggcaagccccgaaggccgtcctgagagtgagacttcctgcctcgtcacaacaacagattttcaaatacagacagaaatggctgcaaccatggagacgtccatatttacaacagagtaccagggtggacatcaccatcaccatcactaataa

SEQ ID NO: 48 - DNA, 1155 bp, synthetic construct
  misc_feature 1..1155: note = Synthesized unprocessed form IL-2 (Gly3)- extracellular domain ofIL-2 R
  source 1..1155: mol_type = other DNA
atggacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgactggtggaggtgagctctgtgacgatgacccgccagagatcccacacgccacattcaaagccatggcctacaaggaaggaaccatgttgaactgtgaatgcaagagaggtttccgcagaataaaaagcgggtcactctatatgctctgtacaggaaactctagccactcgtcctgggacaaccaatgtcaatgcacaagctctgccactcggaacacaacgaaacaagtgacacctcaacctgaagaacagaaagaaaggaaaaccacagaaatgcaaagtccaatgcagccagtggaccaagcgagccttccaggtcactgcagggaacctccaccatgggaaaatgaagccacagagagaatttatcatttcgtggtggggcagatggtttattatcagtgcgtccagggatacagggctctacacagaggtcctgctgagagcgtctgcaaaatgacccacgggaagacaaggtggacccagccccagctcatatgcacaggtgaaatggagaccagtcagtttccaggtgaagagaagcctcaggcaagccccgaaggccgtcctgagagtgagacttcctgcctcgtcacaacaacagattttcaaatacagacagaaatggctgcaaccatggagacgtccatatttacaacagagtaccagggtggacatcaccatcaccatcactaataa

SEQ ID NO: 49 - DNA, 1221 bp, synthetic construct
  misc_feature 1..1221: note = Synthesized unprocessed form IL-2 (Gly4Ser)5- extracellulardomain of IL-2 R
  source 1..1221: mol_type = other DNA
atggacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgactggtggaggtggatcaggtggaggtggatctggtggaggtggatcaggtggaggtggatccggtggaggtggatctgagctctgtgacgatgacccgccagagatcccacacgccacattcaaagccatggcctacaaggaaggaaccatgttgaactgtgaatgcaagagaggtttccgcagaataaaaagcgggtcactctatatgctctgtacaggaaactctagccactcgtcctgggacaaccaatgtcaatgcacaagctctgccactcggaacacaacgaaacaagtgacacctcaacctgaagaacagaaagaaaggaaaaccacagaaatgcaaagtccaatgcagccagtggaccaagcgagccttccaggtcactgcagggaacctccaccatgggaaaatgaagccacagagagaatttatcatttcgtggtggggcagatggtttattatcagtgcgtccagggatacagggctctacacagaggtcctgctgagagcgtctgcaaaatgacccacgggaagacaaggtggacccagccccagctcatatgcacaggtgaaatggagaccagtcagtttccaggtgaagagaagcctcaggcaagccccgaaggccgtcctgagagtgagacttcctgcctcgtcacaacaacagattttcaaatacagacagaaatggctgcaaccatggagacgtccatatttacaacagagtaccagggtggacatcaccatcaccatcactaataa

SEQ ID NO: 50 - protein, 15 aa, synthetic construct
  REGION 1..15: note = (Gly4Ser)3 linker
GGGGSGGGGSGGGGS

SEQ ID NO: 51 - protein, 10 aa, synthetic construct
  REGION 1..10: note = (Gly4Ser)2 linker
GGGGSGGGGS

SEQ ID NO: 52 - protein, 5 aa, synthetic construct
  REGION 1..5: note = (Gly4Ser)1 linker
GGGGS

SEQ ID NO: 53 - DNA, 10 bp, synthetic construct
  misc_feature 1..10: note = Kozak sequence
  source 1..10: mol_type = other DNA
gccaccatgg

SEQ ID NO: 54 - protein, 412 aa, synthetic construct
  REGION 1..412: note = Synthesized unprocessed form of IL-2 (Gly4Ser)4-extracellulardomain of IL-2 R + glycine spacer and poly-histidine region
MDSMQLASCVTLTLVLLVNSAPTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQGGGGSGGGGSGGGGSGGGGSELCLYDPPEVPNATFKALSYKNGTILNCECKRGFRRLKELVYMRCLGNSWSSNCQCTSNSHDKSRKQVTAQLEHQKEQQTTTDMQKPTQSMHQENLTGHCREPPPWKHEDSKRIYHFVEGQSVHYECIPGYKALQRGPAISICKMKCGKTGWTQPQLTCVDEREHHRFLASEESQGSRNSSPESETSCPITTTDFPQPTETTAMTETFVLTMEYKGGHHHHHH

SEQ ID NO: 55 - protein, 417 aa, synthetic construct
  REGION 1..417: note = Synthesized unprocessed form of IL-2 (Gly4Ser)5- extracellulardomain of IL-2 R + glycine spacer and poly-histidine region
MDSMQLASCVTLTLVLLVNSAPTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQGGGGSGGGGSGGGGSGGGGSGGGGSELCLYDPPEVPNATFKALSYKNGTILNCECKRGFRRLKELVYMRCLGNSWSSNCQCTSNSHDKSRKQVTAQLEHQKEQQTTTDMQKPTQSMHQENLTGHCREPPPWKHEDSKRIYHFVEGQSVHYECIPGYKALQRGPAISICKMKCGKTGWTQPQLTCVDEREHHRFLASEESQGSRNSSPESETSCPITTTDFPQPTETTAMTETFVLTMEYKGGHHHHHH

SEQ ID NO: 56 - protein, 408 aa, synthetic construct
  REGION 1..408: note = Synthesized unprocessed form of IL-2 (Gly3Ser)4- extracellulardomain of IL-2 R + glycine spacer and poly-histidine region
MDSMQLASCVTLTLVLLVNSAPTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQGGGSGGGSGGGSGGGSELCLYDPPEVPNATFKALSYKNGTILNCECKRGFRRLKELVYMRCLGNSWSSNCQCTSNSHDKSRKQVTAQLEHQKEQQTTTDMQKPTQSMHQENLTGHCREPPPWKHEDSKRIYHFVEGQSVHYECIPGYKALQRGPAISICKMKCGKTGWTQPQLTCVDEREHHRFLASEESQGSRNSSPESETSCPITTTDFPQPTETTAMTETFVLTMEYKGGHHHHHH

SEQ ID NO: 57 - protein, 404 aa, synthetic construct
  REGION 1..404: note = Synthesized unprocessed form of IL-2 (Gly3Ser)3- extracellular domainof IL-2 R + glycine spacer and poly-histidine region
MDSMQLASCVTLTLVLLVNSAPTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQGGGSGGGSGGGSELCLYDPPEVPNATFKALSYKNGTILNCECKRGFRRLKELVYMRCLGNSWSSNCQCTSNSHDKSRKQVTAQLEHQKEQQTTTDMQKPTQSMHQENLTGHCREPPPWKHEDSKRIYHFVEGQSVHYECIPGYKALQRGPAISICKMKCGKTGWTQPQLTCVDEREHHRFLASEESQGSRNSSPESETSCPITTTDFPQPTETTAMTETFVLTMEYKGGHHHHHH

SEQ ID NO: 58 - protein, 388 aa, synthetic construct
  REGION 1..388: note = Synthesized unprocessed form IL-2 (Gly3Ser)2- extracellulardomain of IL-2 R + glycine spacer and poly-histidine region
MDRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGSGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQGGHHHHHH

SEQ ID NO: 59 - protein, 392 aa, synthetic construct
  REGION 1..392: note = Synthesized unprocessed form of IL-2 (Gly3Ser)3- extracellulardomain of IL-2 R + glycine spacer and poly-histidine region
MDRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGSGGGSGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQGGHHHHHH

SEQ ID NO: 60 - protein, 396 aa, synthetic construct
  REGION 1..396: note = Synthesized unprocessed form IL-2 (Gly3Ser)4- extracellulardomain of IL-2 R + glycine spacer and poly-histidine region
MDRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGSGGGSGGGSGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQGGHHHHHH

SEQ ID NO: 61 - protein, 400 aa, synthetic construct
  REGION 1..400: note = Synthesized unprocessed form IL-2 (Gly4Ser)4- extracellulardomain of IL-2 R + glycine spacer and poly-histidine region
MDRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQGGHHHHHH

SEQ ID NO: 62 - protein, 364 aa, synthetic construct
  REGION 1..364: note = Synthesized mature form IL-2 (Gly3Ser)3- extracellular domain ofmutIL-2 R
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGSGGGSGGGSELCDDDPPEIPHATFKAMAYKEGTMLNCECKRGFTSIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQ

SEQ ID NO: 63 - DNA, 1182 bp, synthetic construct
  misc_feature 1..1182: note = Synthesized unprocessed form IL-2 (Gly3Ser)3- extracellulardomain of mutIL-2 R Mut
  source 1..1182: mol_type = other DNA
atggacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgactggtggaggttctggtggaggttcaggtggaggttcggagctctgtgacgatgacccgccagagatcccacacgccacattcaaagccatggcctacaaggaaggaaccatgttgaactgtgaatgcaagagaggtttcacctcaataaaaagcgggtcactctatatgctctgtacaggaaactctagccactcgtcctgggacaaccaatgtcaatgcacaagctctgccactcggaacacaacgaaacaagtgacacctcaacctgaagaacagaaagaaaggaaaaccacagaaatgcaaagtccaatgcagccagtggaccaagcgagccttccaggtcactgcagggaacctccaccatgggaaaatgaagccacagagagaatttatcatttcgtggtggggcagatggtttattatcagtgcgtccagggatacagggctctacacagaggtcctgctgagagcgtctgcaaaatgacccacgggaagacaaggtggacccagccccagctcatatgcacaggtgaaatggagaccagtcagtttccaggtgaagagaagcctcaggcaagccccgaaggccgtcctgagagtgagacttcctgcctcgtcacaacaacagattttcaaatacagacagaaatggctgcaaccatggagacgtccatatttacaacagagtaccagggtggacatcaccatcaccatcactaataa

SEQ ID NO: 64 - protein, 372 aa, synthetic construct
  REGION 1..372: note = Synthesized mature form IL-2 (Gly4Ser)4- extracellular domain ofmutIL-2 R
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSELCDDDPPEIPHATFKAMAYKEGTMLNCECKRGFTSIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQ

SEQ ID NO: 65 - DNA, 1206 bp, synthetic construct
  misc_feature 1..1206: note = Synthesized unprocessed form IL-2 (Gly4Ser)4- extracellulardomain of mutIL-2 R
  source 1..1206: mol_type = other DNA
atggacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgactggtggaggtggatctggtggaggtggatcaggtggaggtggatccggtggaggtggatctgagctctgtgacgatgacccgccagagatcccacacgccacattcaaagccatggcctacaaggaaggaaccatgttgaactgtgaatgcaagagaggtttcacctcaataaaaagcgggtcactctatatgctctgtacaggaaactctagccactcgtcctgggacaaccaatgtcaatgcacaagctctgccactcggaacacaacgaaacaagtgacacctcaacctgaagaacagaaagaaaggaaaaccacagaaatgcaaagtccaatgcagccagtggaccaagcgagccttccaggtcactgcagggaacctccaccatgggaaaatgaagccacagagagaatttatcatttcgtggtggggcagatggtttattatcagtgcgtccagggatacagggctctacacagaggtcctgctgagagcgtctgcaaaatgacccacgggaagacaaggtggacccagccccagctcatatgcacaggtgaaatggagaccagtcagtttccaggtgaagagaagcctcaggcaagccccgaaggccgtcctgagagtgagacttcctgcctcgtcacaacaacagattttcaaatacagacagaaatggctgcaaccatggagacgtccatatttacaacagagtaccagggtggacatcaccatcaccatcactaataa